(19) United States
(12) Patent Application Publication
(10) Pub. No.: US 2025/0295804 A1
(43) Pub. Date: Sep. 25, 2025

(54) PEG BASED ANTI-CD47/ANIT-PD-L1 BISPECIFIC ANTIBODY-DRUG CONJUGATE

(71) Applicant: Shenzhen Enduring Biotech, Ltd., Shenzhen, Guangdong (CN)

(72) Inventors: Shumin Liu, Shenzhen, Guangdong (CN); Weidong Lyu, Shenzhen, Guangdong (CN); Shuqiang Yin, Shenzhen, Guangdong (CN); Yu Wen, Shenzhen, Guangdong (CN); Dechun Wu, Shenzhen, Guangdong (CN)

(73) Assignee: Shenzhen Enduring Biotech, Ltd., Shenzhen, Guangdong (CN)

(21) Appl. No.: 18/992,608

(22) PCT Filed: Jul. 26, 2023
(86) PCT No.: PCT/CN2023/109315

§ 371 Date: Jan. 9, 2025

(30) Foreign Application Priority Data
Jul. 28, 2022 (WO) PCT/CN2022/108570

(51) Int. Cl. A61K 47/68; A61K 47/60; A61P 35/00

(52) U.S. Cl. CPC A61K 47/6879; A61K 47/60; A61K 47/68031; A61K 47/6851; A61P 35/00

(57) ABSTRACT

An antibody-drug conjugate (ADC) especially PEG based bispecific antibody-drug conjugate (P-BsADC) includes antigen binding domains bonding to CD47 and PD-L1. A method for the preparation of the P-BsADC, a composition including the P-BsADC, and the use thereof in treating diseases.

This application is a U.S. national phase of International Patent Application No. PCT/CN2023/109315, filed on Jul. 26, 2023, which claims the priority of international patent application No. PCT/CN2022/108570 filed on Jul. 28, 2022, the disclosure of which applications are incorporated herein by reference in their entirety.

### REFERENCE TO SEQUENCE LISTING

The present application is being filed along with a Sequence Listing in electronic format. The Sequence Listing is provided as a file entitled 145954.05000-Sequence Listing.xml, created on Dec. 26, 2024, which is 8,818,688 bytes in size. The information in the electronic format of the sequence listing is incorporated herein by reference in its entirety.

### FIELD OF INVENTION

The present invention relates to an polyethyleneglycol (PEG) based antibody-drug conjugate (ADC), especially PEG based bispecific antibody-drug conjugate (P-BsADC) targeting two different receptors of tumor cells. In particular, the invention relates to a long acting PEGylated single chain bispecific antibody drug conjugate targeting at CD47 and PD-L1.

### BACKGROUND OF INVENTION

Traditional small molecule cytotoxic drugs for killing rapid dividing cells have been widely used in cancer treatment for decades, but the non-specific action of such agents could also attack the proliferating healthy cells, resulting in chemotherapy associated toxicity and side effect (Baah, S et al. Molecules, 2021, 26). Although monoclonal antibodies could differentiate tumors much better from healthy tissues, they are not as potent as small molecule cytotoxic drugs (Shefet-Carasso, L. et al. Drug Resist Update, 2015, 18, 36-46). Antibody drug conjugates (ADC) have been developed to take the advantages of both potent cytotoxic agents and the capabilities of tumor antigen recognition of antibodies (Khongorzul, P. et al. Molecular Cancer Research, 2020, 18, 3-19). Currently, there are 12 ADCs approved by FDA in the U.S., and more than 100 candidates of ADCs currently active in clinical trials (Coats, S. et al. Clin. Cancer Res., 2019, 25, 5441-5448). Unfortunately, all approved ADCs showed severe adverse effect and very often the dosages used for receiving clinical efficacies are very close to their maximum tolerated dose (MTD), resulting in very narrow therapeutic windows (Beck, A. et al. Nat. Rev. Drug Discov., 2017, 16, 315-337; Vankemmelbeke, M. et al. Ther. Deliv., 2016, 7, 141-144; Tolcher A. W. et al. Ann. Oncol., 2016, 27, 2168-2172). Furthermore, the toxicity profiles and dose-limiting toxicities of ADCs are typically associated with cytotoxic warheads (Fu, Z. et al. Signal Transduction and Targeted Therapy, 2022, 7, 93).

There are also some inherited toxicities directly associated with the design and the structure of ADC. For instance, ADC toxicity could result from the off-target/off-tumor binding to Fc receptors (FcγRs) or lectin receptors (such as the mannose receptor) on normal cells (Donaghy, H. et al. MAbs, 2016, 8, 659-671). Regularly, Fc on the antibody will not induce such toxicities, but the ADC, on the other hand, will kill the FcγRs or mannose expressing cells due to the release of cytotoxic payload inside of the cells (Gorovits, B. et al. Cancer Immunol Immunother, 2013, 62, 217-223). Another Fc dependent toxicity results from the ADC aggregates, which can activate Fcγ receptors on immune cells, internalize via FcγRs, ultimately kill such target-negative cells (Aoyama, M. et al. Pharmaceutical Research, 2022, 39, 89-103). Obviously, there are multiple mechanisms of ADC toxicity that are Fc dependent.

ADCs require efficient internalization and trafficking to lysosomes to be efficacious, the effluxing of the internalized traditional ADC before reaching lysosomes will offset the internalization efficiency, and partly accounts for the closeness of the clinical dose and MTD. To overcome this issue, a biparatopic ADC has been developed to significantly increase efficiency of internalization, decrease efflux and enhance tumor inhibition efficacy (DaSilva, J. O. et al. Clinical Cancer Research, 2022, 26, 1408-1419; DaSilva, J. O. et al. Molecular Cancer Therapeutics, 20, 1966-1976; Gauzy-Lazo, L. et al. SLAS Discov., 2020, 25, 843-868). Furthermore, bispecific ADC has been developed to increase tumor selectivity and could be engineered for multiple mechanisms of action to synergistically improve efficacy (Kast, F. et al. Nature Communications, 2021, 12, 3790; Maruani, A. Drug Discov Today Technol, 2018, 30, 55-61).

Antibody drugs including ADC are faced with several barriers that impact intratumoral distribution. The primary means of antibody transport inside tumors is based on diffusion, which is influenced by antibody size, binding affinity, tumor microenvironment, vascularization, and availability of targeted antigen (Xenaki, K. T. et al. Front Immunol, 2017, 8, 1287). The large size of antibody or ADC with molecule weight around 150 kd makes it hard to extravasate the blood vessels to deep penetrate tumor tissue, small size antibody fragments showed significantly increased tumor biodistribution (Li, Z. et al. MAbs, 2016, 8, 113-119). Binding site barrier (BSB) is another obstacle for antibody to penetrate tumor (Miao, L. et al. ACS Nano, 2016, 10, 9243-9258). Because the high affinity of the antibody to cellular target is the main reason for the binding site barrier, to improve efficacy of ADC T-DM1 in solid tumors, a strategy through transient competitive inhibition of antibody-antigen binding showed promising results (Bordeau, B. M. et al. Cancer Res. 2021, 81, 4145-4154). In a research of co-administering a non-conjugated competitive antibody with ADC, it was found that the effect of binding site barrier is decreased and the ADC is more homogenously distributed (Evans, R. et al. Sci Rep., 2022, 12, 7677).

Recently, antibody therapies with anti-PD-1 or anti-PD-L1 enjoy significant clinical success as well as market success. In a normal situation. PD-L1/PD-1 signaling pathway is one of the immune suppressive mechanisms to prevent autoimmunity, unfortunately it was utilized by tumor cells to evade immune surveillance. Thus, blocking this signaling pathway by anti-PD-1 or anti-PD-L1 and reinvigorating immunity could be used for cancer therapy (Han, Y. et al. Am J Cancer Res., 2020, 10, 727-742). As PD-L1 (not PD-1) antigen is expressed on tumor cells, it could be used as a ADC target. It is reported that PD-L1 is highly expressed in almost all types of hematologic cancer and solid tumors. For example, PD-L1 is reported to be expressed in up to 100% of melanoma tumor samples, up to 95% of NSCLC tumors, up to 54% RCC tumors, up to 89% in ovarian cancer, up to 93% in multiple myeloma, etc (Patel, S. P. et al. Mol Cancer Ther., 2015, 14, 847-856; Gandini, S., et al. Critical reviews in oncology/hematology, 2016, 100, 88-98). On the other hand, PD-L1 is also expressed in normal cells and tissues, such as T. B, antigen-presenting cells and in some non-lymphoid tissues, and detected in the cardiac endothelium, placenta, and pancreatic islets as well (Qin, W. et al. Front Immunol, 2019, 10, 2298). Therefore, there could be a challenge for developing traditional ADC to target PD-L1.

Furthermore, anti-PD-L1 single agent therapies could restore latent anti-tumor immunity and generate clinical response of 43% in melanoma, and approximately 20% in advanced NSCLC2 (Mahoney, K. M. et al. Clin Ther., 2015, 37, 764-782; Valecha, G. K. et al. Expert review of anticancer therapy, 2017, 17, 47-59; Malhotra, J. et al. Translational lung cancer research, 2017, 6, 196-211, Qiao, M. et al. Clinical lung cancer, 2017, 06.005; Emens, L. A. et al. European journal of cancer, 2017, 81, 116-129). It is evident that some patients do not respond to anti-PD-L1 agents even though the tumor specimens are PD-L1 positive (Qiao, M. et al. Clinical lung cancer, 2017, 06.005; Emens, L. A. et al. European journal of cancer, 2017, 81, 116-129; Wang, Q. & Wu, X. International immunopharmacology, 46, 210-219). For some cancer patients responded to anti-PD-L1 therapy initially, they may also acquire resistance and the disease will progress after the initial response (Pathak, R. et al. Cancers (Basel), 2020, 12). It is reported that the resistance mechanisms relate to additional immune suppressive signaling or neoantigen mutation (Lei, Q. et al. Front Cell Dev Biol, 2020, 8, 672).

CD47/SIRPα signaling pathway is another immune checkpoint that attracts researcher's attention recently. CD47 is a component of innate immune checkpoint on tumor cells and functions as a “do not eat me” signal through interacting with its receptor signal regulatory protein alpha (SIRPα) on professional phagocytic cells (e.g. macrophage and neutrophil). Like PD-L1, CD47 antigen is also overexpressed on tumor cells in almost all cancer types (Willingham, S. B., et al., 2012. Proc Natl Acad Sci USA. 109 (17), p 6662-7; Chao. M. P., et al., 2012, Current opinion in immunology, 24 (2), p 225-232). The overexpression of CD47 is associated with poor prognosis or recurrence in clinic settings (Chan, K. S., et al., 2009, Proc Natl Acad Sci USA, 106 (33), p 14016-21; Yuan, J., et al., 2019, Oncol Lett, 18 (3), p 3249-3255; Majeti, R., et al., 2009. Cell, 138 (2), p 286-99). CD47 is broadly expressed at low levels on many normal cells, yet in certain types of normal cells, such as T cells, NK, red blood cells, and platelets and the like (Strizova. Z., et al., 2020, Scientific reports, 10 (1), p 13936-13936; Olsson, M., et al., 2005, Blood, 105 (9), p 3577-82), CD47 are expressed at high levels. The high expression levels of CD47 have brought huge challenge for developing antibody agents to block CD47/SIRPα.

This invention will provide a novel PEG based single chain bispecific antibody drug conjugate to address the afore-mentioned problems.

### SUMMARY OF THE INVENTION

This invention provides a PEG-based bispecific antibody drug conjugate prepared by site-specific conjugation of PEGylated drug conjugate to a bispecific antibody fragment or a single chain bispecific antibody with an engineered site or sites for site-specific conjugation.

In one aspect, the invention provides a conjugate of Formula I:

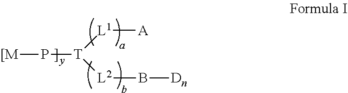

wherein
P can be a non-immunogenic polymer;
M can be a proton or a terminal capping group selected from C₁₋₅₀ alkyl and aryl, wherein one or more carbons of said alkyl are optionally replaced with a heteroatom;
y can be an integer selected from 1, 2, 3, 4, 5, 6, 7, 8, 9 and 10;
T can be a multi-functional linker having two or more functional groups, wherein the linkage between T and (L¹)ₐ and the linkage between T and (L²)_{b} can be the same or different;
Each of L¹ and L² can be independently a bifunctional linker;
Each of a and b can be an integer selected from 0, 1, 2, 3, 4, 5, 6, 7, 8, 9 and 10;
B can be a branched linker, wherein each branch can have an amino acid sequence or a trigger moiety cleavable by an enzyme, a pH liable linker that can release the drug D or its derivative at acidic pH conditions, or a disulfide bond linker that can release the drug D by enzymatic cleavage;
A can be any form of bispecific antibody or antigen binding protein including a single chain bispecific antibody, a bispecific nanobody or other bispecific antigen binding fragment thereof that targeting at CD47 and PD-L1;
D can be any cytotoxic small molecule or peptide or derivative thereof and can be released from B through either enzymatic hydrolysis and/or self-immolating mechanism or pH induced hydrolysis or any combination mechanisms thereof; each D can be the same or different;
n can be an integer selected from 1, 2, 3, 4, 5, 6, 7, 8, 9, 10, 11, 12, 13, 14, 15, 16, 17, 18, 19, 20, 21, 22, 23, 24 and 25.

Another aspect of the invention provides a conjugate of Formula II:

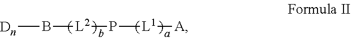

wherein each of the variables are as defined for Formula I.

In some embodiments, each branch of B comprises an extension spacer (optional), a trigger moiety, e.g. an amino acid sequence or a disulfide moiety or a carbohydrate moiety such as β-glucoronide or β-galactoside, connected to a drug D via one or more self-immolating spacer, cleavable by e.g. cathepsins B, plasmin, matrix metalloproteinases (MMPs), glutathione, thioredoxin family members (WCGH/PCK), thio reductase (Arunachalam, B. et. al. Proc. Natl. Acad Sci. USA, 2000, 97, 745-750). Examples of self-immolating spacers include but not limit to the following:

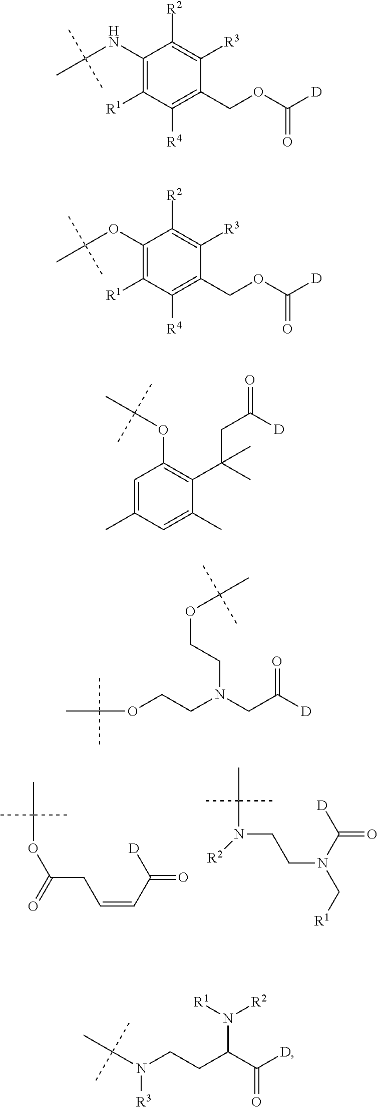

wherein R¹, R², R³, R⁴ can be H, or C₁₋₁₀ alkyl. In such embodiments, D can be any small molecule or peptide or derivative thereof containing active O or N or S functional group.

Other examples of one or two self-immolating spacers include but not limit to the following:

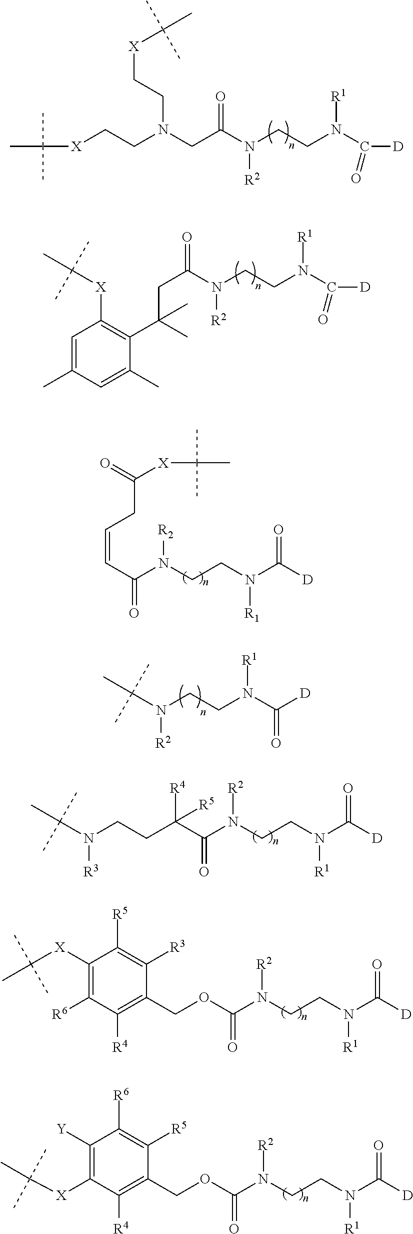

wherein n is 1 or 2; Y is a carbohydrate moiety; R¹, R², R³, R⁴, R⁵, R⁶ can be H, or C₁₋₁₀ alkyl or —(CH₂CH₂—O)₁₋₁₀—CH₃ or any combination thereof and X=O, S or N. In such embodiments, D can be any small molecule or peptide or derivative thereof containing active —OH functional group that is linked to the self-immolating spacer.

In some embodiments, each branch of B can be a pH liable linker that can release the drug D or its derivatives at acidic pH conditions at tumor site and/or inside of the tumor cell. Examples of acidic liable linkers include but not limit to the following formats;
—CR¹═N—NR¹—, —CR¹═N—O—, —CR¹═N—NR²—CO—, —N═N—CO—, —OCOO—, —NR¹—COO—.

In some embodiments, each branch of B can be a disulfide bond linker that can release the drug D or its derivatives at tumor site and/or inside of the tumor cell by enzymatic cleavage.

In some embodiments, A is a single chain anti-CD47/anti-PD-L1 bispecific antibody that binds to CD47 and PD-L1 expressed on cancer cells.

In some embodiments, D is monomethyl auristatin E (MMAE), an antimitotic drug or its derivative, or SN38, a potent topoisomerase I inhibitor or its derivative or a combination thereof.

In a further embodiment, D is MMAE and is connected to a self-immolating spacer such as 4-aminobenzyl alcohol through a carbonate (PABC) and a trigger moiety such as Valine-Citrulline.

In any of the above aspects and embodiments, the non-immunogenic polymer can be selected from the group consisting of polyethylene glycol (PEG), dextrans, carbohydrate polymers, polyalkylene oxide, polyvinyl alcohols, hydroxypropyl-methacrylamide (HPMA), and a co-polymer thereof. Preferably, the non-immunogenic polymer is PEG, such as a branched PEG or a linear PEG. The total molecule weight of the PEG can be ranged from 3000 to 100,000 Daltons, e.g., 5000 to 80,000, 10,000 to 60,000, and 20,000 to 40,000 Daltons. The PEG can be linked to a multifunctional moiety either through a permanent bond or a cleavable bond.

Functional group for site-specific conjugation that forms linkage between (L), and protein A can be selected from the group consisting of thiol, maleimide, methylsulfonyl pyrimidin, methylsulfonyl benzothiazole, vinylpyridine, ethyl P-ethynyl-N-(p-tolyl) phosphonamidate, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid, alkyne, Iodine, and the like.

In some embodiments, one of (L¹)ₐ can comprise a linkage formed from azide and alkyne or from methylsulfonyl pyrimidin and thiol or maleimide and thiol. In some embodiments, the alkyne can be dibenzocyclooctyl (DBCO).

In some embodiments, T can be lysine, P can be PEG, and y can be 1, while the alkyne can be dibenzocyclooctyl (DBCO).

In some embodiments, A can be derived from an azide tagged bispecific antibody including a single chain bispecific antibody, a bispecific nanobody or other bispecific antigen binding fragment thereof, or a combination thereof targeting CD47 and PD-L1, wherein the azide can be conjugated to an alkyne in the respective (L¹)ₐ. In other embodiments, protein A can be derived from a thiol tagged bispecific antibody including a single chain bispecific antibody, a bispecific nanobody or other a bispecific antigen binding fragment thereof, or a combination thereof targeting CD47 and PD-L1, wherein the thiol can be conjugated to a maleimide or methylsulfonyl pyrimidin in the respective (L¹)ₐ.

The above-described bispecific antibody drug conjugate can be made according to a method comprising: (i) preparing a non-immunogenic polymer drug conjugate with a terminal functional group that is capable of site-specific conjugation to an bispecific antibody or its modified form; and (ii) site-specific conjugating the non-immunogenic polymer drug conjugate to an bispecific antibody or its modified structure to form a compound of Formula I or II. In some examples, the bispecific antibody can be modified with a small molecule linker before the conjugation step.

The invention also provides a pharmaceutical formulation comprising the above-described bispecific antibody drug conjugate e.g. PEGylated bispecific single chain antibody drug conjugate and a pharmaceutically acceptable carrier.

The invention further provides a method of treating a disease in a subject in need thereof comprising administering an effective amount of the above-described bispecific antibody drug conjugate e.g. PEGylated bispecific single chain antibody drug conjugate.

The details of one or more embodiments of the invention are set forth in the description below. Other features, objectives, and advantages of the invention will be apparent from the description and from the claims.

Preferred embodiments of the invention are as follows.

1. A compound of the Formula (I)

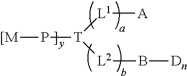

wherein
P is a non-immunogenic polymer;
M is a proton or a terminal capping group selected from C₁₋₅₀ alkyl and aryl, wherein one or more carbons of said alkyl are optionally replaced with a heteroatom;
y is an integer selected from 1 to 10;
A is a bispecific antibody or antigen binding fragment thereof targeting two different antigens selected from tumor specific antigens (TSA) and tumor associated antigens (TAA);
T is a trifunctional small molecule linker moiety;
each of L¹ and L² is independently a hetero or homobifunctional linker;
each of a and b is an integer selected from 0-10;
B is a branched linker, wherein each branch has an amino acid sequence or carbohydrate moiety linked to a self-immolating spacer, wherein cleavage of the amino acid sequence or carbohydrate moiety by an enzyme triggers self-immolating mechanism to release D. or each branch has a disulfide bond, wherein cleavage of the disulfide bond releases D or its derivative, or each branch has a cleavable bond, wherein cleavage of the cleavable bond with certain cleavage mechanism releases D;
each of D is independently a cytotoxic small molecule or peptide; and
n is an integer selected from 1-25.

2. The compound of embodiment 1, wherein the functional group at the linker terminal of L¹ is capable of site-specific conjugation with A, and is selected from the group consisting of thiol, maleimide, methylsulfonyl pyrimidin, methylsulfonyl benzothiazole, vinylpyridine, ethyl P-ethynyl-N-(p-tolyl) phosphonamidate, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine.

3. The compound of any of embodiments 1-2, wherein the bispecific antibody is a single chain bispecific antibody, a bispecific nanobody, or a bispecific antigen binding domain thereof.

4. The compound of embodiment 3, wherein the bispecific antibody comprises an antigen-binding domain binding to CD47 comprising a light chain variable region (VL) and a heavy chain variable region (VH) and an antigen-binding domain binding to PD-L1 comprising a VL and a VH.

5. The compound of any one of embodiments 1-4, wherein the bispecific antibody is a single chain anti-CD47/anti-PD-L1 bispecific antibody.

6. The compound of embodiment 4 or 5, wherein the VL of the antigen-binding domain binding to CD47 comprises CDR1, CDR2 and CDR3 as shown in SEQ ID Nos. 2-4 respectively, and the VH of the antigen-binding domain binding to CD47 comprises CDR1, CDR2 and CDR3 as shown in SEQ ID Nos. 5-7 respectively; and
the VL of the antigen-binding domain binding to PD-L1 comprises CDR1, CDR2 and CDR3 as shown in SEQ ID Nos. 8-10 respectively, and the VH of the antigen-binding domain binding to PD-L1 comprises CDR1, CDR2 and CDR3 as shown in SEQ ID Nos. 11-13 respectively.

7. The compound of embodiment 6, wherein the VL of the antigen-binding domain binding to CD47 comprises an amino acid sequence as shown in SEQ ID No. 15 or an amino acid sequence having at least 80%, at least 85%, at least 90%, at least 95%, at least 98%, or at least 99% sequence identity to SEQ ID No. 15, and the VH of the antigen-binding domain binding to CD47 comprises an amino acid sequence as shown in SEQ ID No. 16 or an amino acid sequence having at least 80%, at least 85%, at least 90%, at least 95%, at least 98%, or at least 99% sequence identity to SEQ ID No. 16; and
the VL of the antigen-binding domain binding to PD-L1 comprises an amino acid sequence as shown in SEQ ID No. 18 or an amino acid sequence having at least 80%, at least 85%, at least 90%, at least 95%, at least 98%, or at least 99% sequence identity to SEQ ID No. 18, and the VH of the antigen-binding domain binding to PD-L1 comprises an amino acid sequence as shown in SEQ ID No. 17 or an amino acid sequence having at least 80%, at least 85%, at least 90%, at least 95%, at least 98%, or at least 99% sequence identity to SEQ ID No. 17.

8. The compound of any of embodiments 4-7, wherein the bispecific antibody has an amino acid sequence as shown in SEQ ID NO: 1 or 14 or an amino acid sequence having at least 80%, at least 85%, at least 90%, at least 95%, at least 98%, or at least 99% sequence identity to SEQ ID No. 1 or 14.

9. The compound of any of embodiments 4-8, wherein the antigen-binding domain binding to CD47 and the antigen-binding domain binding to PD-L1 are linked via a peptide linker or other chemical linker, and wherein the linker comprises a cysteine, an aide or an unnatural amino acid residue for site-specific conjugation of the bispecific antibody to L¹.

10. The compound of any one of embodiments 1-9, wherein D is selected from any DNA crosslinker agent, microtubule inhibitor, DNA alkylator, topoisomerase inhibitor or a combination thereof.

11. The compound of embodiment 10, wherein D is selected from MMAE, MMAF, SN38, DM1, DM4, calicheamycins, pyrrolobenzodiazepines, duocarmycins or a derivate thereof, or a combination thereof.

12. The compound of any one of embodiments 1-11, wherein the non-immunogenic polymer is polyethylene glycol (PEG).

13. The compound of embodiment 12, wherein the PEG is a liner PEG or a branched PEG.

14. The compound of embodiment 12 or 13, wherein at least one terminal of the polyethylene glycol is capped with methyl or a low molecule weight alkyl.

15. The compound of any of embodiments 12-14, wherein a total molecule weight of the PEG is from 3000 to 100000.

16. The compound of any one of embodiments 12-15, wherein the PEG is linked to a cyclic or noncyclic trifunctional moiety T (e.g. a lysine) through a permanent bond or a cleavable bond.

17. A compound of the Formula (II)

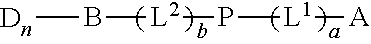

wherein
P is a liner PEG;
A is a bispecific antibody or antigen binding fragment thereof targeting two different tumor specific antigens (TSA) or tumor associated antigens (TAA);
each of L¹ and L² is independently a bifunctional linker;
each of a and b is an integer selected from 0-10;
B is a branched linker, wherein each branch has an amino acid sequence or carbohydrate moiety linked to a self-immolating spacer, wherein cleavage of the amino acid sequence or carbohydrate moiety by an enzyme triggers self-immolating mechanism to release D, or each branch has a disulfide bond, wherein cleavage of the disulfide bond releases D or its derivative, or each branch has a cleavable bond, wherein cleavage of the cleavable bond with certain cleavage mechanism releases D;
each of D is independently a cytotoxic small molecule or peptide;
n is an integer selected from 1-25.

18. The compound of embodiment 17, wherein the functional group at the linker terminal of L¹ is capable of site-specific conjugation with A, and is selected from the group consisting of thiol, maleimide, methylsulfonyl pyrimidin, methylsulfonyl benzothiazole, vinylpyridine, ethyl P-ethynyl-N-(p-tolyl) phosphonamidate, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine.

19. The compound of embodiment 17 or 18, wherein the bispecific antibody is a single chain bispecific antibody, a bispecific nanobody, or a bispecific antigen binding domain thereof.

20. The compound of embodiment 19, wherein the bispecific antibody comprises an antigen-binding domain binding to CD47 comprising a VL and a VH and an antigen-binding domain binding to PD-L1 comprising a VL and a VH.

21. The compound of any one of embodiments 17-20, wherein the bispecific antibody is a single chain anti-CD47/anti-PD-L1 bispecific antibody.

22. The compound of embodiment 20 or 21, wherein the VL of the antigen-binding domain binding to CD47 comprises CDR1, CDR2 and CDR3 as shown in SEQ ID Nos. 2-4 respectively, and the VH of the antigen-binding domain binding to CD47 comprises CDR1. CDR2 and CDR3 as shown in SEQ ID Nos. 5-7 respectively; and
the VL of the antigen-binding domain binding to PD-L1 comprises CDR1, CDR2 and CDR3 as shown in SEQ ID Nos. 8-10 respectively, and the VH of the antigen-binding domain binding to PD-L1 comprises CDR1, CDR2 and CDR3 as shown in SEQ ID Nos. 11-13 respectively.

23. The compound of embodiment 22, wherein the VL of the antigen-binding domain binding to CD47 comprises an amino acid sequence as shown in SEQ ID No. 15 or an amino acid sequence having at least 80%, at least 85%, at least 90%, at least 95%, at least 98%, or at least 99% sequence identity to SEQ ID No. 15, and the VH of the antigen-binding domain binding to CD47 comprises an amino acid sequence as shown in SEQ ID No. 16 or an amino acid sequence having at least 80%, at least 85%, at least 90%, at least 95%, at least 98%, or at least 99% sequence identity to SEQ ID No. 16; and
the VL of the antigen-binding domain binding to PD-L1 comprises an amino acid sequence as shown in SEQ ID No. 18 or an amino acid sequence having at least 80%, at least 85%, at least 90%, at least 95%, at least 98%, or at least 99% sequence identity to SEQ ID No. 18, and the VH of the antigen-binding domain binding to PD-L1 comprises an amino acid sequence as shown in SEQ ID No. 17 or an amino acid sequence having at least 80%, at least 85%, at least 90%, at least 95%, at least 98%, or at least 99% sequence identity to SEQ ID No. 17.

24. The compound of any of embodiments 20-23, wherein the bispecific antibody has an amino acid sequence as shown in SEQ ID NO: 1 or 14 or an amino acid sequence having at least 80%, at least 85%, at least 90%, at least 95%, at least 98%, or at least 99% sequence identity to SEQ ID No. 1 or 14.

25. The compound of any of embodiments 20-24, wherein the antigen-binding domain binding to CD47 and the antigen-binding domain binding to PD-L1 are linked via a peptide linker or other chemical linker, and wherein the linker comprises a cysteine, an azide or an unnatural amino acid residue for site-specific conjugation of the bispecific antibody to L¹.

26. The compound of any one of embodiments 17-25, wherein D is selected from any DNA crosslinker agent. Microtubule inhibitor, DNA alkylator, Topoisomerase inhibitor or a combination thereof.

27. The compound of embodiment 26, wherein D is selected from MMAE, MMAF, Dxd, SN38, DM1, DM4, calicheamycins, pyrrolobenzodiazepines, duocarmycins or a derivate thereof, or a combination thereof.

28. The compound of any of embodiments 17-27, wherein a total molecule weight of the PEG is from 3000 to 100000.

29. The compound of embodiment 28, wherein the PEG is linked to any cyclic or noncyclic trifunctional or multifunctional moiety T (e.g. a lysine) through a permanent bond or a cleavable bond.

30. The compound of embodiment 1 or 17 selected from the formula:

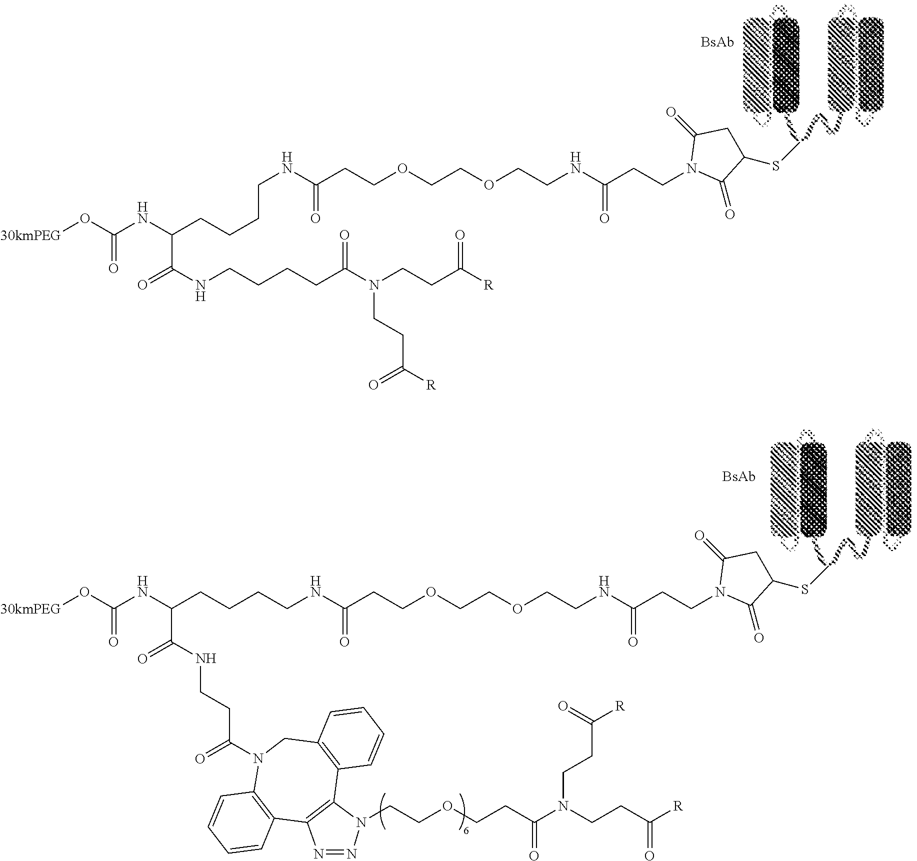

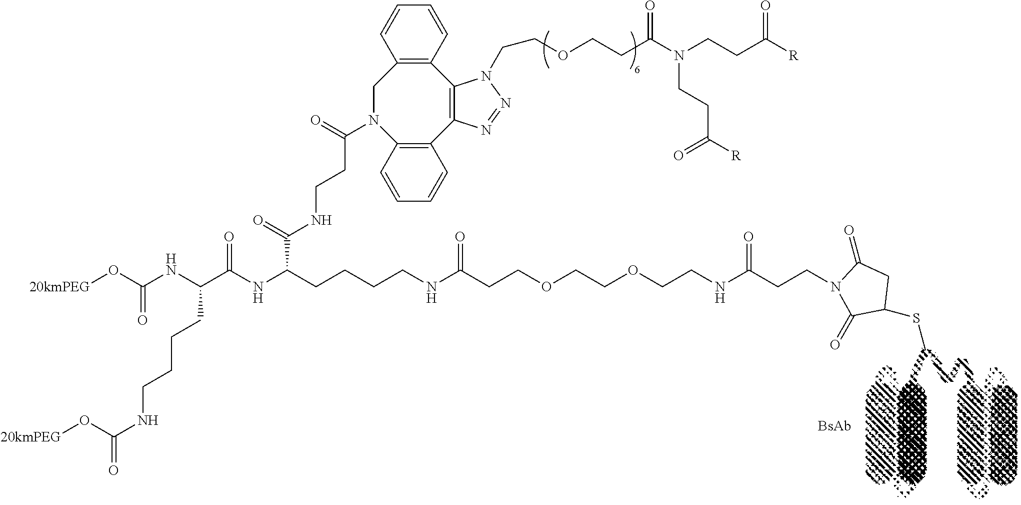

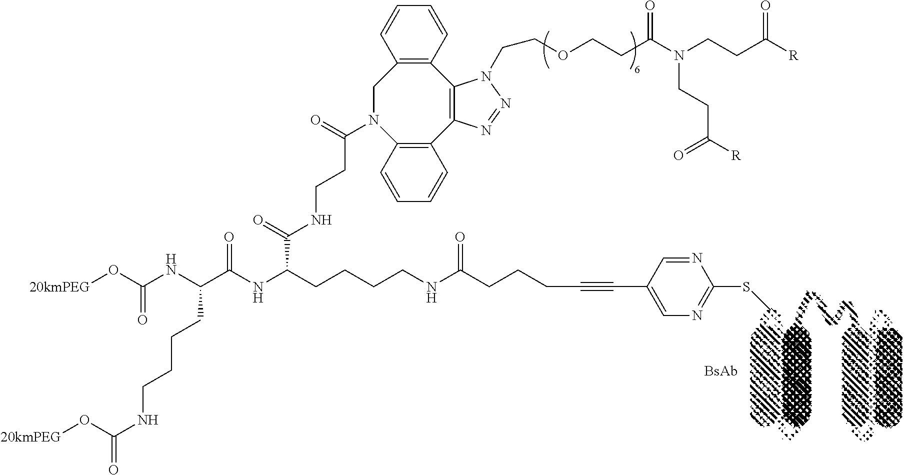

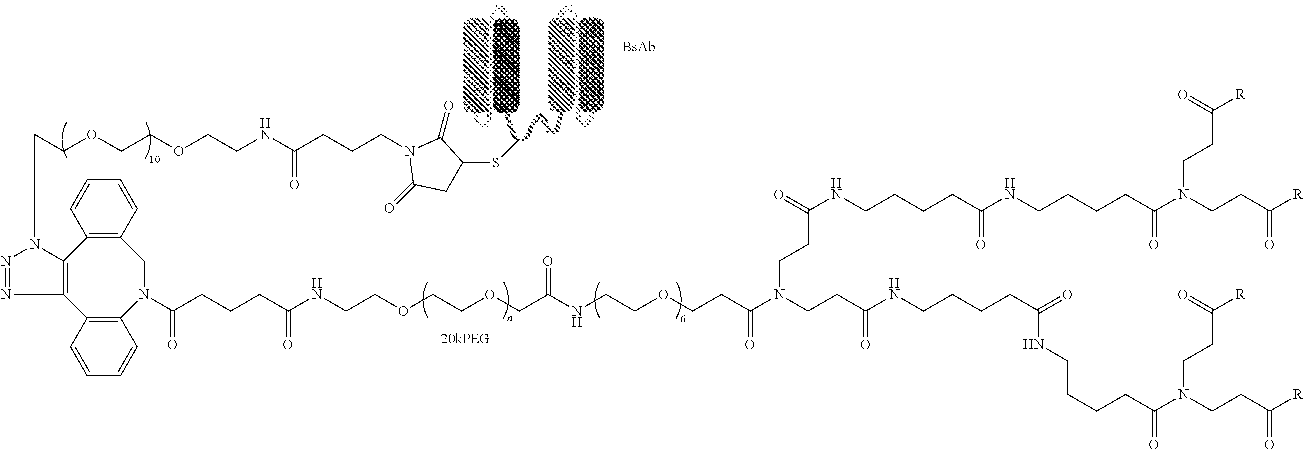

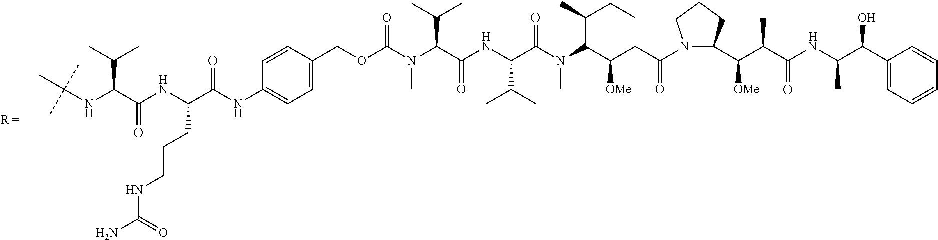

or a pharmaceutically acceptable salt thereof;
wherein BsAb is a bispecific antibody targeting PD-L1 and CD47 or an antigen binding fragment thereof.

31. The compound of embodiment 30, wherein the bispecific antibody comprises
an antigen-binding domain binding to CD47 comprising a VL comprising CDR1, CDR2 and CDR3 as shown in SEQ ID Nos. 2-4 respectively, and a VH comprising CDR1, CDR2 and CDR3 as shown in SEQ ID Nos. 5-7 respectively; and
an antigen-binding domain binding to PD-L1 comprising a VL comprising CDR1, CDR2 and CDR3 as shown in SEQ ID Nos. 8-10 respectively, and a VH comprising CDR1, CDR2 and CDR3 as shown in SEQ ID Nos. 11-13 respectively.

32. The compound of embodiment 31, wherein the VL of the antigen-binding domain binding to CD47 comprises an amino acid sequence as shown in SEQ ID No. 15 or an amino acid sequence having at least 80%, at least 85%, at least 90%, at least 95%, at least 98%, or at least 99% sequence identity to SEQ ID No. 15, and the VH of the antigen-binding domain binding to CD47 comprises an amino acid sequence as shown in SEQ ID No. 16 or an amino acid sequence having at least 80%, at least 85%, at least 90%, at least 95%, at least 98%, or at least 99% sequence identity to SEQ ID No. 16; and
the VL of the antigen-binding domain binding to PD-L1 comprises an amino acid sequence as shown in SEQ ID No. 18 or an amino acid sequence having at least 80%, at least 85%, at least 90%, at least 95%, at least 98%, or at least 99% sequence identity to SEQ ID No. 18, and the VH of the antigen-binding domain binding to PD-L1 comprises an amino acid sequence as shown in SEQ ID No. 17 or an amino acid sequence having at least 80%, at least 85%, at least 90%, at least 95%, at least 98%, or at least 99% sequence identity to SEQ ID No. 17.

33. The compound of any of embodiments 30-32, wherein the bispecific antibody has an amino acid sequence as shown in SEQ ID No: 1 or 14 or an amino acid sequence having at least 80%, at least 85%, at least 90%, at least 95%, at least 98%, or at least 99% sequence identity to SEQ ID No. 1 or 14.

34. A method of preparing a compound of any one of embodiments 1-33, comprising:
a) a step of preparation of the non-immunogenic modified (e.g. PEGylated) drug conjugate with a free functional group for site-specific conjugation;
b) a step of site-specific conjugation of the non-immunogenic modified (e.g. PEGylated) drug conjugate to a bispecific antibody to provide a compound of the Formula (I) or (II).

35. A pharmaceutical formulation comprising an effective amount of the compound of any one of embodiments 1-33 and a pharmaceutically acceptable salt, carrier or excipient.

36. A compound of any one of embodiments 1 to 33 for use in the treatment of a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias. T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer.

37. A compound of any one of embodiments 1 to 33 for use in combination with an effective amount of another anticancer agent or immunosuppressant agent in the treatment of a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer.

### DESCRIPTION OF THE DRAWINGS

FIG. 1. Schematically illustrates a reaction scheme of preparing compound 7 described in Example 1.

FIG. 2. Schematically illustrates a reaction scheme of preparing compound 13 described in Example 1.

FIG. 3. Schematically illustrates a reaction scheme of preparing compound 18 described in Example 1.

FIG. 4. Schematically illustrates a reaction scheme of preparing compound 22 described in Example 1.

FIG. 4a. Schematically illustrates a reaction scheme of preparing compound 22a described in Example 1.

FIG. 5. Schematically illustrates a reaction scheme of preparing compound 27 described in Example 1.

FIG. 6. Schematically illustrates a reaction scheme of preparing compounds 32 and 32a described in Example 1a.

FIG. 7. Schematically illustrates a reaction scheme of preparing compound 35 described in Example 1b.

FIG. 7a. Schematically illustrates a reaction scheme of preparing compound 35a described in Example 1c.

FIG. 7b. Schematically illustrates a reaction scheme of preparing compound 35b described in Example 1d.

FIG. 7c. Schematically illustrates a reaction scheme of preparing compound 35c described in Example 1e.

FIG. 8. Schematically illustrates a reaction scheme of preparing compound 39 described in Example 1f.

FIG. 9. Schematically illustrates a reaction scheme of preparing compound 41 described in Example 1g.

FIG. 10. Schematically illustrates a reaction scheme of preparing JY207 described in Example 3.

FIG. 10a. Schematically illustrates a reaction scheme of preparing JY207A described in Example 3a.

FIG. 10b. Schematically illustrates a reaction scheme of preparing JY207A1 described in Example 3b.

FIG. 10c. Schematically illustrates a reaction scheme of preparing JY207B described in Example 3c.

FIG. 10d. Schematically illustrates a reaction scheme of preparing JY207W described in Example 4.

FIG. 11. Cell line selectivity of JY207 described in Example 5.

FIG. 12. JY207 induces cytotoxicity to tumor cells lines expressing both CD47 and PD-L1 described in Example 6.

FIG. 12a. JY207A selectively induces strong cytotoxicity to tumor cells lines expressing both CD47 and PD-L1 with no cytotoxicity to CD47+/PD-L1− MKN45 cells and CD47−/PD-L1+NCIN87 cells described in Example 6a.

FIG. 12b. JY207A1 selectively induces strong cytotoxicity to tumor cells lines expressing both CD47 and PD-L1 with no cytotoxicity to CD47+/PD-L1− MKN45 cells and CD47−/PD-L1+NCIN87 cells described in Example 6a.

FIG. 12c. JY207B selectively induces strong cytotoxicity to tumor cells lines expressing both CD47 and PD-L1 with no cytotoxicity to CD47+/PD-L1− MKN45 cells and CD47−/PD-L 1+NCIN87 cells described in Example 6a.

FIG. 13. Comparison of internalization efficiency between JY207 and DS8201a described in Example 7.

FIG. 14. Selective binding of tumor cell line with JY207 described in Example 8.

FIG. 15. In vivo efficacy of JY207 on pancreatic cancer xenograft tumor model described in Example 9.

FIG. 16. In vivo efficacy of JY207 on NCI-H1975 xenograft tumor model described in Example 10.

### DETAILED DESCRIPTION OF THE INVENTION

In this invention, a single chain anti-PD-L1/anti-CD47 bispecific antibody is used as the antibody module for constructing PEG-base ADC to target at cancer cells that coexpress the two antigens (PD-L1 and CD47).

For normal cells (such as erythrocytes) that express only CD47, the regular anti-CD47/anti-PD-L1 bispecific ADC may theoretically still be able to recognize and bind CD47 on erythrocytes. Yet, since the anti-CD47/anti-PD-L1 bispecific ADC in this invention is a pegylated single chain bispecific ADC, its affinity for CD47 will be significantly reduced (inside data) due to the structure of single chain antibody and steric hindrance of pegylation. Therefore, the PEG based single chain bispecific ADCs will be safe to such normal cells. In fact, it is very rare that normal cells express both PD-L1 and CD47, and co-expression of these two antigens most often occurs on cancer cells. Therefore, the mechanism of action and the structural characteristics determine that the pegylated single chain anti-CD47/anti-PD-L1 bispecific ADC only kills cancer cells and does not harm normal cells.

Both CD47 and PD-L1 are overexpressed in many cancer types. Although not many data of the coexpression of these two proteins in the same tumor tissue can be found, it is expected that the proportion of tumors that co-express both proteins in the clinic will not be low. In a study involving 430 non-small cell lung cancer clinical samples, 96 (22.3%) expressed PD-L1 and 296 (68.8%) expressed CD47, of which 80 (18.6%) showed PD-L1/CD47 co-expression, and the PD-L1/CD47 co-expression rate in LUSC cancer subtypes was 23.7%, which was significantly higher than 14.6% in LUAD cancer subtypes (p=0.018) (Yang, Z. et al. Thorac Cancer, 2021, 12, 1743-1751). In a study involving 148 lung sarcoid-like cancers, 54 (36.5%) and 78 (52.7%) were PD-L1-positive and CD47-positive, respectively, of which 36 (24.3%) showed a co-expression of PD-L1 and CD47 (Yang, Z., et al. J Cancer Res Clin Oncol, 2019, 145, 3055-3065). The coexpression rate in almost all cancer types will grant the pegylated single chain antiCD47/antiPD-L1 bispecific ADC to benefit many patients with many different cancer types.

One critical disadvantage of traditional antibody-based drugs including traditional ADC is their poor tumor penetration due to the large molecular size (150 kDa). As a result, the antibodies may exert limited effect within solid tumors, leading to suboptimal efficacy. Some investigators are trying to screen low-molecular weight anti-PD-L1 peptides for cancer immunotherapy (Liu, H. et al. Journal for ImmunoTherapy of Cancer, 2019, 7, 270), but such compound typically will have very short half-life. In this invention, the molecule weight (MW) of the pegylated single chain bispecific ADC is around 80-85 kd, which is much smaller than traditional antibody based ADCs, but large enough to maintain good half-life up to a week. Therefore, this invention will have the advantage in improved penetration of solid tumors.

Furthermore, since the mechanism of action for pegylated single chain anti-PD-L1/anti-CD47 bispecific ADC does not rely on the signaling blockage of PD-L1/PD-1 or CD47/SIRPα, PD-L1 and CD47 only are used as tumor specific antigens to guide the specific binding and release the payload to exert cytotoxicity to the cancer cells. Thus, the PEG based single chain anti-PD-L1/anti-CD47 bispecific ADC could be used to address the drug resistance issues associated with anti-PD-L1 and anti-CD47 agents.

In summary, this invention provides a novel PEGylated single chain bispecific antibody drug conjugate that not only shows no toxicity to megakaryocytes and other normal cells due to no Fc component, which often induces Fc associated toxicities with traditional full length ADCs, but also increases the selectivity toward tumor cells and enhances the anti-tumor effect of the conjugate. Accordingly, this invention expands current ADC technologies and improves current cancer therapy.

### I. Conjugate

In one aspect of the invention, compounds of formula I and 11 are provided:

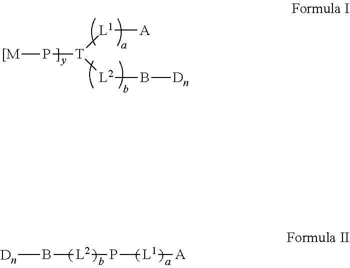

In the conjugate of Formula I or Formula II, P can be a non-immunogenic polymer such as a PEG;
M can be a proton or a terminal capping group selected from C₁₋₅₀ alkyl and aryl, wherein one or more carbons of said alkyl are optionally replaced with a heteroatom;
y can be an integer selected from 1, 2, 3, 4, 5, 6, 7, 8, 9, 10;
T can be a moiety having two or more functional groups, wherein the linkage between T and (L¹)ₐ and the linkage between T and (L²)_{b} can be the same or different;
Each of L¹ and L² can be independently a bifunctional linker;
Each of a and b can be an integer selected from 0, 1, 2, 3, 4, 5, 6, 7, 8, 9, 10;
B can be a branched linker, wherein each branch can be an amino acid sequence or a trigger moiety cleavable by an enzyme, a pH liable linker that can release the drug D or its derivatives at acidic pH conditions, or a disulfide bond linker that can release the drug D by enzymatic cleavage;
A can be any bispecific antibody including a single chain bispecific antibody, a bispecific nanobody or other bispecific antigen binding fragment, or combination of thereof, targeting CD47 and PD-L1;
D can be any cytotoxic small molecule or peptide or derivative thereof and can be released from B through either enzymatic hydrolysis and/or self-immolating mechanism or pH induced hydrolysis or any combination thereof; each D can be the same or different;
n can be an integer selected from 1, 2, 3, 4, 5, 6, 7, 8, 9, 10, 11, 12, 13, 14, 15, 16, 17, 18, 19, 20, 21, 22, 23, 24 and 25.

In some embodiments, each branch of B comprises an extension spacer (optional), a trigger moiety, e.g. an amino acid sequence or a disulfide moiety or a carbohydrate moiety such as β-glucoronide or D-galactoside, connected to drug D via one or more self-immolating spacer, cleavable by e.g. cathepsins B, plasmin, matrix metalloproteinases (MMPs), glutathione, thioredoxin family members (WCGH/PCK), thio reductase (Arunachalam, B. et. al. Proc. Natl. Acad Sci. USA, 2000, 97, 745-750). Examples of self-immolating spacers include but not limit to the following:

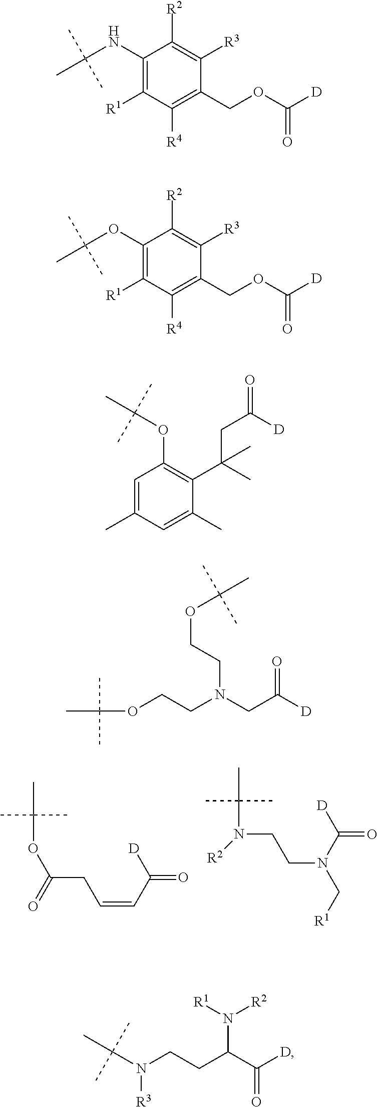

wherein R¹, R², R³, R⁴ can be H, or C₁₋₁₀ alkyl. In such embodiments, D can be any small molecule or peptide or derivative thereof containing active O or N or S functional group.

Other examples of one or two self-immolating spacers include but not limit to the following:

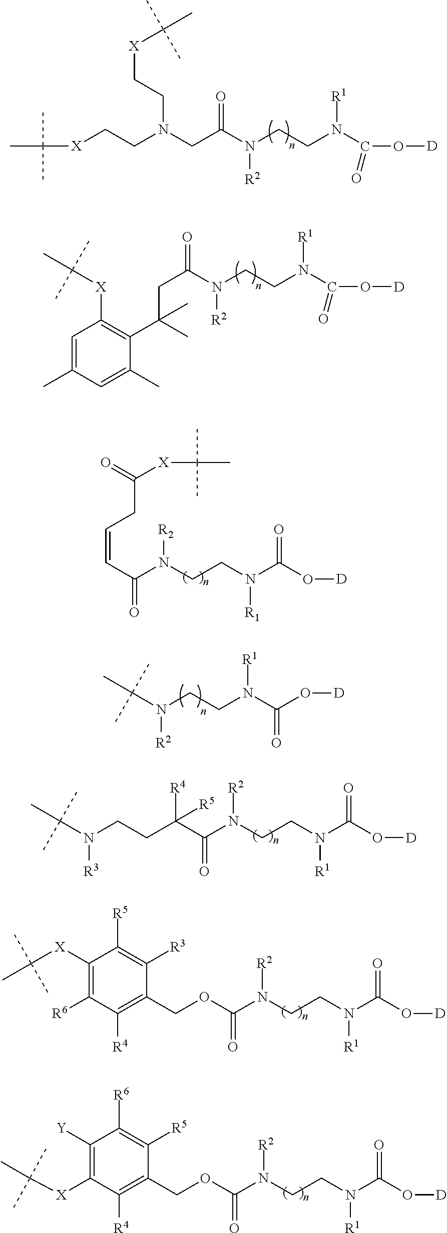

wherein n is 1 or 2; Y is a carbohydrate moiety; R¹, R², R³, R⁴, R⁵, R⁶ can be H, or C₁₋₁₀ alkyl or —(CH₂CH₂—O)₁₋₁₀—CH₃ or any combination thereof and X=O, S or N. In such embodiments, D can be any small molecule or peptide or derivative thereof containing free —OH functional group.

In some embodiments, each branch of B can be a pH liable linker that can release the drug D or its derivatives at acidic pH conditions at tumor site and/or inside of the tumor cell. Examples of acidic liable linkers include but not limit to the following formats:
—CR¹═N—NR¹—, —CR¹═N—O—, —CR¹═N—NR²—CO—, —N═N—CO—, —OCOO—, —NR¹—COO—.

In some embodiments, each branch of B can be a disulfide bond linker that can release the drug D or its derivatives at tumor site and/or inside of the tumor cell by enzymatic cleavage.

In some embodiments, A is a single chain anti-CD47/anti-PD-L1 bispecific antibody that binds to the CD47 and PD-L1 expressed on cancer cells.

In some embodiments, A is a single chain bispecific antibody (SCAPD-L1/SCACD47) that is able to bind to two different antigens such as PD-L1 and CD47.

In some embodiments, the six complementarity-determining regions (CDRs) targeting CD47 could be:

(LCDRI, SEQ ID No. 2)
RSSQSIVYSNGN,
(LCDR2, SEQ ID No. 3)
KVSNRFS,
(LCDR3, SEQ ID No. 4)
FQGSHVPYT,
(HCDRI, SEQ ID No. 5)
NYNMH,
(HCDR2, SEQ ID No. 6)
TIYPGNDDTSYNQKFK,
and
(HCDR3, SEQ ID No. 7)
GGYRAMDY.

In some embodiments, the six complementarity-determining regions (CDRs) targeting PD-L1 could be:

(LCDR1, SEQ ID No. 8)
GFTFSDSWIH,
(LCDR2, SEQ ID No. 9)
AWISPYGGSTYYADSVKG,
(LCDR3, SEQ ID No. 10)
RHWPGGFDY,
(HCDR1, SEQ ID No. 11)
RASQDVSTAVA,
(HCDR2, SEQ ID No. 12)
SASFLYS,
and
(HCDR3, SEQ ID No. 13)
QQYLYHPAT.

In some embodiments, amino acid sequence of a VL targeting CD47 could be:

(SEQ ID No. 15)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIK.

In some embodiments, amino acid sequence of a VH targeting CD47 could be:

(SEQ ID NO. 16)
QVQLVQSGAEVKKPGASVKVSCKASGYTFTNYNMHWVRQAPGORLEWMGT
IYPGNDDTSYNQKFKDRVTITADTSASTAYMELSSLRSEDTAVYYCARGG
YRAMDYWGQGTLVTVSS.

In some embodiments, amino acid sequence of a VH targeting PD-L1 could be:

(SEQ ID NO. 17)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK.

In some embodiments, amino acid sequence of a VL targeting PD-L1 could be:

(SEQ ID NO. 18)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVT.

In some embodiments, amino acid sequence of SCAPD-L1/SCACD47 could be:

(SEQ ID NO. 1)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKGGSGGSGGSGGSGGQVQLVQSGAEVKKPGASVKVSCKA
SGYTFTNYNMHWVRQAPGQRLEWMGTIYPGNDDTSYNQKFKDRVTITADT
SASTAYMELSSLRSEDTAVYYCARGGYRAMDYWGQGTLVTVSSGCGGSSG
GSDIQMTQSPSSLSASVGDRVTITCRASQDVSTAVAWYQQKPGKAPKLLI
YSASFLYSGVPSRFSGSGSGTDFTLTISSLQPEDFATYYCQQYLYHPATF
GQGTKVEIKGGGGSGGGGSGGGGSEVQLVESGGGLVQPGGSLRLSCAASG
FTFSDSWIHWVRQAPGKGLEWVAWISPYGGSTYYADSVKGRFTISADTSK
NTAYLQMNSLRAEDTAVYYCARRHWPGGFDYWGQGTLVT.

In some embodiments, amino acid sequence of SCAPD-L1/SCACD47 could be:

(SEQ ID NO. 14)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKGGSGGSGGSGGSGGQVQLVQSGAEVKKPGASVKVSCKA
SGYTFTNYNMHWVRQAPGQRLEWMGTIYPGNDDTSYNQKFKDRVTITADT
SASTAYMELSSLRSEDTAVYYCARGGYRAMDYWGQGTLVTVSSGCGGSSG
GSDIQMTQSPSSLSASVGDRVTITCRASQDVSTAVAWYQQKPGKAPKLLI
YSASFLYSGVPSRFSGSGSGTDFTLTISSLQPEDFATYYCQQYLYHPATF
GQGTKVEIKGGGGSGGGGSGGGGSEVQLVESGGGLVQPGGSLRLSCAASG
FTFSDSWIHWVRQAPGKGLEWVAWISPYGGSTYYADSVKGRFTISADTSK
NTAYLQMNSLRAEDTAVYYCARRHWPGGFDYWGQGTLVTHHHHHH.

In some embodiments, D can be released either at tumor site or inside of tumor cells by either enzymatic and/or self-immolating mechanism or PH induced hydrolysis.

In some embodiments, drug D can be selected from any DNA crosslinker agent, microtubule inhibitor. DNA alkylator, topoisomerase inhibitor or a combination thereof.

In some embodiments, drug D can be selected from monomethyl auristatin E (MMAE), monomethyl auristatin F (MMAF), maytansinoids (DM1, DM4), SN38, Dxd, calicheamicins, pyrrolobenzodiazepines, sibiromycin, tomaymycin, duocarmycins or any cytotoxic compounds or their analogs/derivates thereof, or a combination thereof.

In some embodiments, D is monomethyl auristatin E (MMAE), an antimitotic drug or its derivative.

In a further embodiments, D is connected to a self-immolating spacer such as 4-aminobenzyl alcohol through a carbonate group (PABC) and a trigger moiety such Valine-Citrulline to form Val-Cit-PABC-D.

In one aspect of this invention, methods for preparing PEGylated drug conjugate that is capable of site-specific conjugating to a bispecific antibody fragment or single chain bispecific antibody are provided. In another aspect of this invention, methods for preparing PEGylated single chain BsADC are provided.

To synthesize PEGylated single-chain BsADC targeting CD47 and PD-L1, coding sequence or a vector carrying a coding sequence of single-chain bispecific antibody can be synthesized and introduced into, e.g., the CHO expression systems. The proteins can be expressed and purified as described previously (WO2018075308).

For the synthesis of PEGylated drug conjugate with a side chain that has a site-specific conjugation functional group, a terminal functional group of PEG such as hydroxyl or carboxyl group and the like can be activated and conjugated with a trifunctional small molecule moiety such as Boc or Fmoc protected lysine to form a terminal branched heterobifunctional PEG. The newly formed carboxyl group can be coupled with a branch spacer to form PEG-Lys(Boc)-B. After coupling, the protection group can be removed, and the unprotected PEGylated branch linker can be coupled with a small molecule linker that has site-specific conjugation functional group such as maleimide, 2-Methylsulfonyl pyrimidin or DBCO to form PEG-Lys(Mal)-B or PEG-Lys(pyrimidin)-B or PEG-Lys(DBCO)-B. The PEGylated drug conjugate such as PEG-lys(Mal)-B-Val-Cit-PABC-MMAE can be prepared by coupling reaction of PEG-Lys(MaW)-B with Val-Cit-PABC-MMAE. The final step of synthesis is site-specific conjugation of PEGylated drug conjugate to a thiol or azide tagged single chain bispecific antibody.

Alternatively, Val-Cit-PABC-MMAE can react with B to form B(Val-Cit-PABC-MMAE)n followed by conjugation with PEG-Lys(Mal)-COOH to have PEGylated drug conjugate PEG-lys(Mal)-B(Val-Cit-PABC-MMAE)n, wherein n is an integer from 1 to 20.

### II. Polyethylene Glycol (PEG) Moiety

In one embodiment of the present invention, the linear PEG can be of the formula:

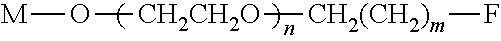

In the formula, n can be an integer from 60 to about 2300 to preferably provide a polymer having a total molecule weight of from 3000 to 100000 Dalton or greater if desired. M can be H, methyl or other low molecule weight alkyl. Non-limiting examples of M include H, methyl, ethyl, isopropyl, propyl, butyl or F₁(CH₂)_{q}CH₂. F and F₁ can be independently a terminal functional group such as hydroxyl, carboxyl, thiol, halide, amino group and the like, which is capable of being functionalized, activated and/or conjugated to a small molecule spacer or linker. q and m can be any integer from 0 to 10.

In another embodiment of present invention, the method can also be carried out with an alternative branched PEG. The branched PEG can be of the formula:

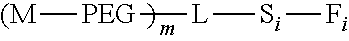

In this formula, PEG is polyethylene glycol. m can be an integer between 2 to 10 to preferably provide a branched PEG having a total molecule weight of from 3000 to 100000 Dalton or greater if desired. M can be methyl or other low molecule weight alkyl cap. L can be a functional linkage moiety to that two or more PEGs are attached. Non-limiting examples of such linkage moiety are: any amino acids such as glycine, alanine, lysine, or 1,3-diamino-2-propanol, triethanolamine, any 5 or 6 member aromatic or aliphatic rings with more than two functional groups attached. S is any non-cleavable spacer. F can be a terminal functional group such as hydroxyl, carboxyl, thiol, amino group. i is 0 or 1. When i equals to 0, the formula is shown as:

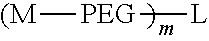

wherein: each variables of PEG, m, M or L have the same definitions as above.

The method of the present invention can also be carried out with alternative polymeric substances such as dextrans, carbohydrate polymers, polyalkylene oxide, polyvinyl alcohols or other similar non-immunogenic polymers, the terminal groups of which are capable of being functionalized or activated. The foregoing list is merely illustrative and not intended to restrict the type of non-antigenic polymer suitable for use herein.

### III. Trifunctional Linker T

T represents a multifunctional linker such as a trifunctional linker, connecting with P, (L¹)ₐ and (L²)_{b}. T can be derived from molecules with any combination of three functional groups, non-limiting examples of which include hydroxyl, amino, hydrazinyl, carboxyl, thiol, and halide. The functional groups in a trifunctional linker may be the same or different. In some embodiments, one or two of the functional groups may be protected to achieve selective conjugation with other reaction partners. A variety of protecting groups are known in the art, including for example, those shown in Advanced Organic Chemistry by March (Third Edition, 1985, Wiley and Sons, New York). A functional group may also be converted into other groups before or after the reaction between T and another reaction partner. For example, a hydroxyl group may be converted into a mesylate or a tosylate group. A halide may be replaced with an azido group. An acid functional group of T may be converted to an alkyne function group by coupling with an amino group bearing a terminal alkyne.

In exemplary embodiments, T is derived from lysine, aspartic acid, glutamic acid, 1,3-diamino-2-propanol, or triethanolamine. One or more of the functional groups on these molecules may be protected for selective reactions. In some embodiments, T is derived from a Boc-protected lysine.

IV. Bifunctional Linker L¹ and L²

Both linkers L¹ and L² comprise linker chains that may be independently selected from

—(CH₂)ₐXY(CH₂)_{b}—,

—X(CH₂)ₐO(CH₂CH₂O)_{c}(CH₂)_{b}Y—,

-heterocyclyl-,

—(CH₂)ₐX—,

—X(CH₂)ₐY—,

—W₁—(CH₂)ₐC(O)NR₁(CH₂)_{b}O(CH₂CH₂O)_{c}(CH₂)_{d}X—,

—X(CH₂)ₐO(CH₂CH₂O)_{b}(CH₂)_{c}W₂C(O)(CH₂)_{d}Y—,

—W₃—(CH₂)ₐC(O)NR₁(CH₂)_{b}O(CH₂CH₂O)_{c}(CH₂)_{d}W₂C(O)(CH₂)ₑX—,

—C≡C—,

—CR₁═CR₂—,

wherein a, b, c, d and e are each an integer independently selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; each of X and Y is independently selected from C(═O), NR₂, S, O, CR₃R₄ or Null; R₁, R₂, R₃ and R₄ independently represent hydrogen, C1-10 alkyl or (CH₂)₁₋₁₀C(═O); W₁ and/or W₃ is derived from a maleimido-based moiety, methylsulfonyl pyrimidin-based moiety, methylsulfonyl benzothiazole-based moiety, 4-vinylpyridine-based moiety, ethyl P-ethynyl-N-phenylphosphonamidate-based moiety; W₂ represents a triazolyl or a tetrazolyl containing group, the heterocyclyl group is selected from a maleimido-derived moiety or a tetrazolyl-based or a triazolyl-based moiety. Non-limiting examples of a maleimido-based moiety include N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), K-maleimidoundecanoic acid N-succinimidyl ester (KMUA), γ-maleimidobutyric acid N-succinimidyl ester (GMBS), ε-maleimidcaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). Alternatively, the heterocyclyl linkage group of the linker may be pyrimidin, 4-vinylpyridine, tetrazolyl or triazolyl.

In some exemplary embodiments, (L¹)ₐ and (L²)_{b} can be selected from:

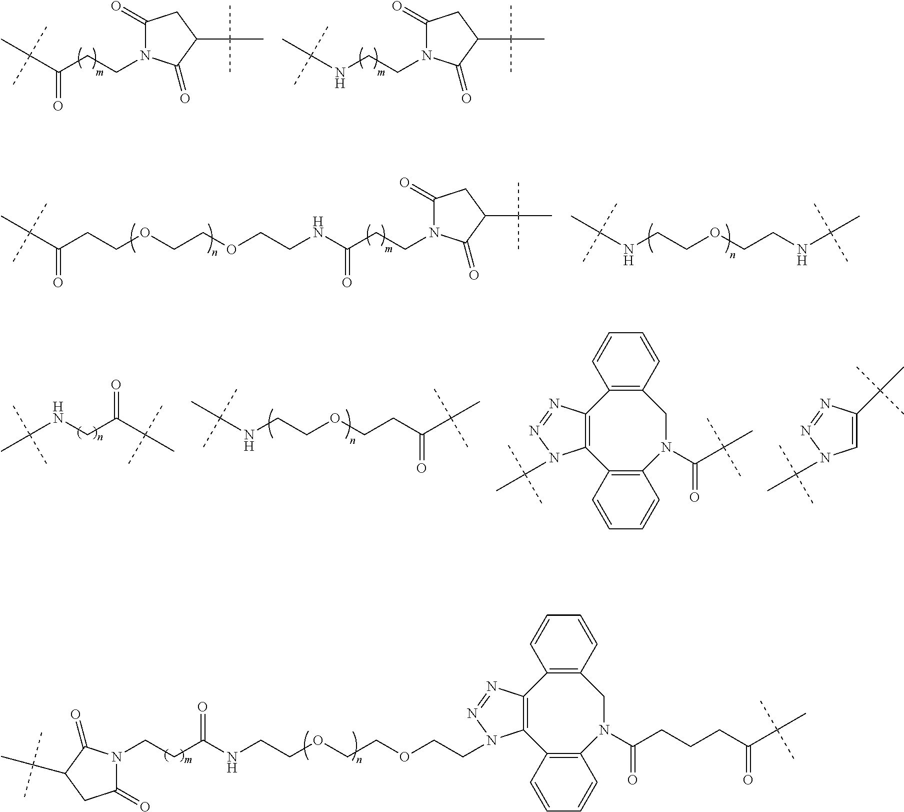

wherein n and m are integer and independently selected from 0 to 20.

In other exemplary embodiments, each linker unit can also be derived from a haloacetyl-based moiety selected from N-succinimidyl-4-(iodoacetyl)-aminobenzoate (SIAB), N-succinimidyl iodoacetate (SIA), N-succinimidyl bromoacetate (SBA), or N-succinimidyl 3-(bromoacetamido)propionate (SBAP).

In some non-limiting exemplary embodiments, each linker unit can also be derived from a sulfone-based moiety selected from vinyl sulfone reagent, mono-sulfone reagent and bis-sulfone reagent.

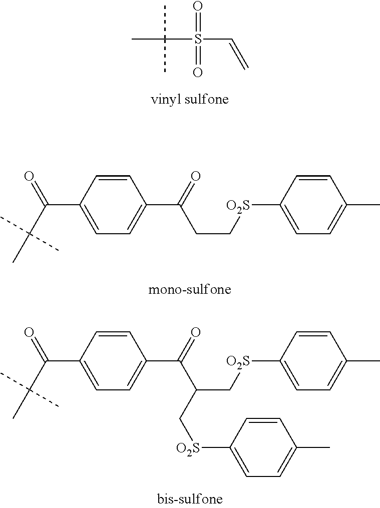

In other non-limiting exemplary embodiments, each linker unit can also be derived from following moieties:

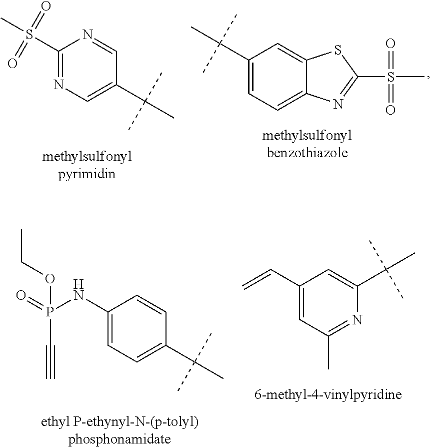

### V. Branched Linker B

The branched linker B can comprise a branching unite, an extension spacer (optional), a trigger unit, one or more self-immolating spacer or any combination of such.

In some embodiments, a branching unite comprises structures that may be independently selected from:

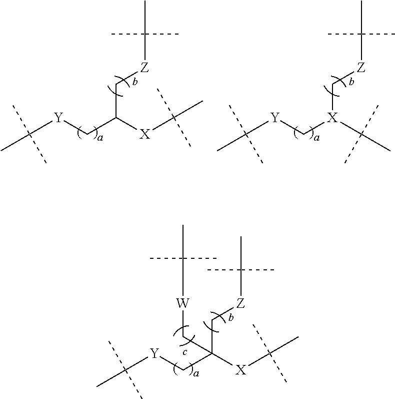

1. X, Y, Z or W=NR¹, NR², C(═O), O, N or null, wherein R¹ and R² independently represent hydrogen or C₁₋₁₀ alkyl group
2. a, b, c is integer from 0-10

In other embodiments, a branching unite comprises structures that may be independently selected from:

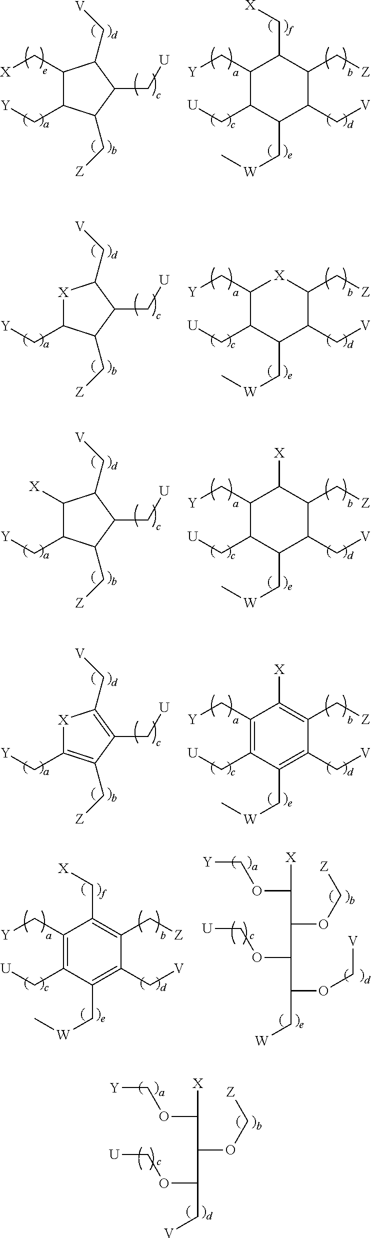

1. X, Y, Z, U, V, W=C(O), NR¹, NR², O, N or Null, wherein R¹ and R² independently represent hydrogen or C1-10 alkyl group
2. a, b, c, d, e=0-10

In some embodiments, an extension spacer in each branch comprises linker chains that may be independently selected from:
—X(CH₂)ₐO(CH₂CH₂O)_{b}(CH₂)_{c}Y—, —X(CH₂)ₐY—,
   or any combination thereof, wherein a, b, and c are each an integer selected from 0 to 25, all subunits included; X and Y may be selected independently from NR¹, NR², C(O), O, or Null, wherein R¹ and R² independently represent hydrogen or C₁₋₁₀ alkyl group.

In some embodiments, a branching unit (e.g. with two branches) with or without extension spacers can be joined by two or more branching units (e.g. with two branches) to form a branching unit with four branches.

In other embodiments, a trigger unit comprises any amino acid sequence or any carbohydrate moiety or a disulfide bond or a PH liable bond or any cleavable bond that can be enzymatically or chemically cleaved.

In some embodiments, a self-immolating spacer comprises structures that may be selected from:

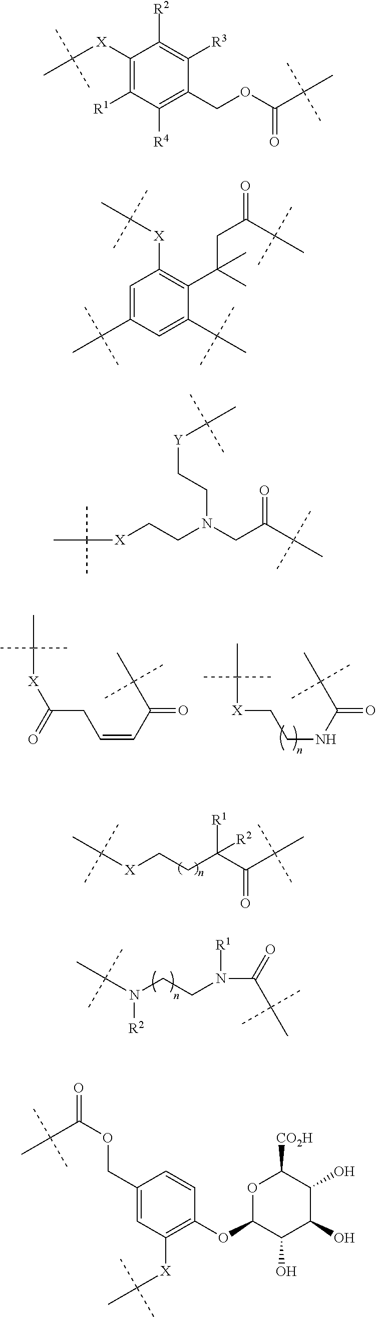

wherein n is 1 or 2, R¹, R², R³ and R⁴ independently represent hydrogen, C₁₋₁₀ alkyl or —(CH₂CH₂)ₘCH₃, wherein m=1-10, X and Y can be NH or O or S.

In some embodiment, two self-immolating spacers can be connected to each other, e.g.

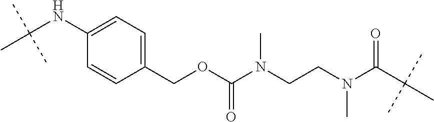

In some embodiments, the branched linker B can be selected from:

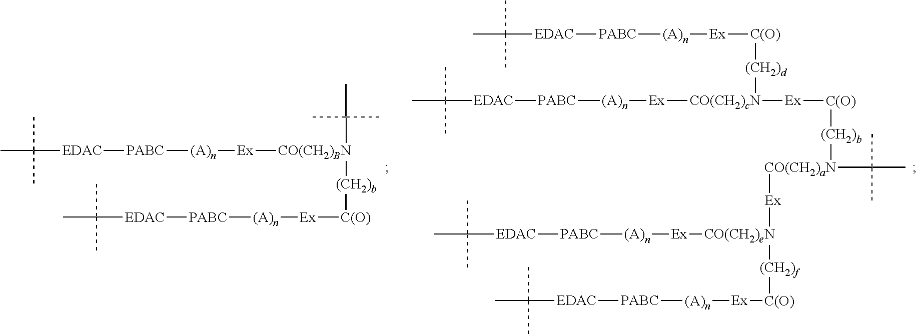

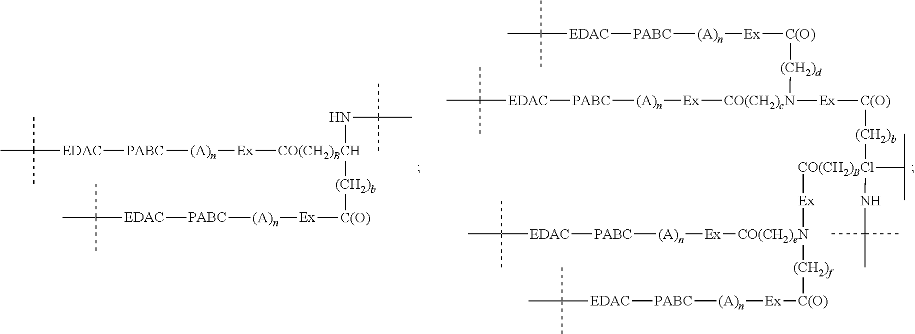

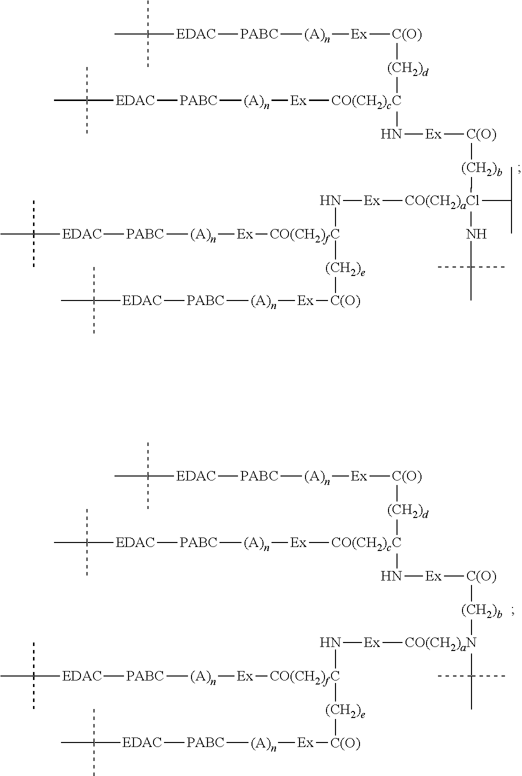

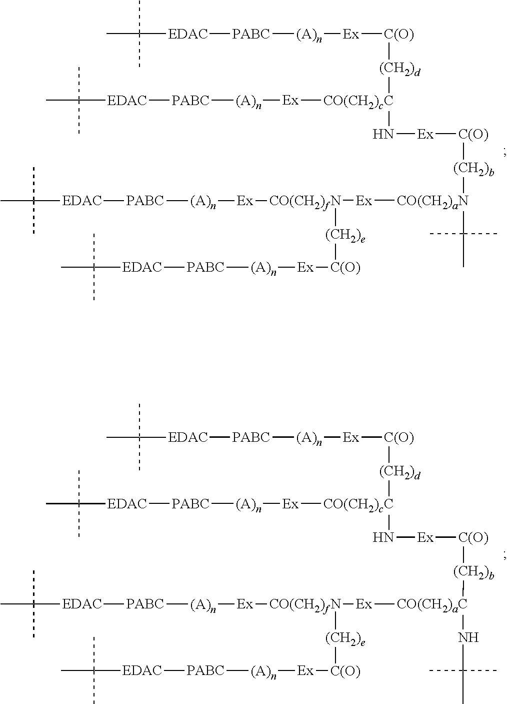

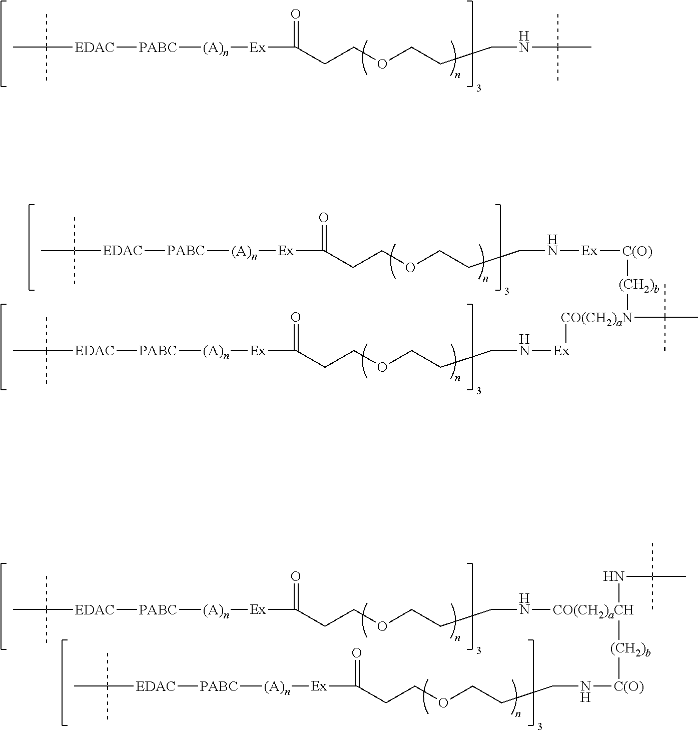

Wherein:
each of a, b, c, d, e and f is independently an integer selected from 1-25;
n is an integer selected from 1 to 10,
(A)ₙ is a trigger unit of amino acid sequence, each A is an independent amino acid and n is any integer from 1-25;
PABC is 4-aminobenzyl alcohol carbonate;
EDAC could be null or —CONR¹CH₂CH₂NR²— or —CONR¹CH₂CH₂CH₂NR²—, wherein R¹ and R² independently represent hydrogen, C₁₋₁₀ alkyl group or —(CH₂CH₂O)ₘCH₃, wherein m is any integer from 1-10;
Ex is an extension spacer that comprises linker chains that may be independently selected from:

—NR¹(CH₂)ₐO(CH₂CH₂O)_{b}(CH₂)_{c}C(O)—,

—C(O)(CH₂)ₐNR¹—,

—NR¹(CH₂)ₐO(CH₂CH₂O)_{b}(CH₂)_{c}NR²—,

—NR¹(CH₂)ₐNR²—,

—NR¹(CH₂)ₐO(CH₂CH₂O)_{b}(CH₂)_{c}O—,

—O(CH₂)ₐNR¹—,

—C(O)(CH₂)ₐO—,

—O(CH₂)ₐO(CH₂CH₂O)_{b}(CH₂)_{c}C(O)—,

—C(O)(CH₂)ₐO(CH₂CH₂O)_{b}(CH₂)_{c}C(O)—,

—C(O)(CH₂)ₐC(O)
or Null:
   wherein a, b, and c are each an integer selected from 0 to 25, all subunits included; and R¹ and R² independently represent hydrogen or C₁₋₁₀ alkyl group.

In some other embodiments, the trigger unit of the amino acid sequence can be Val-Cit, Val-Ala, Val-Lys, Phe-Lys, Phe-Cit, Phe-Arg, Phe-Ala, Ala-Lys, Leu-Cit, Ile-Cit, Trp-Cit, D-Phe-Phe-Lys, Phe-Phe-Lys, Gly-Phe-Lys, Gly-Phe-Leu-Gly, or Ala-Leu-Ala-Leu, Gly-Gly-Phe-Gly.

For preferred embodiments, the amino acid sequence can be Val-Cit, Phe-Lys, Gly-Gly-Phe-Gly or Val-Lys.

In some exemplary embodiments, branched linker B can be selected from:

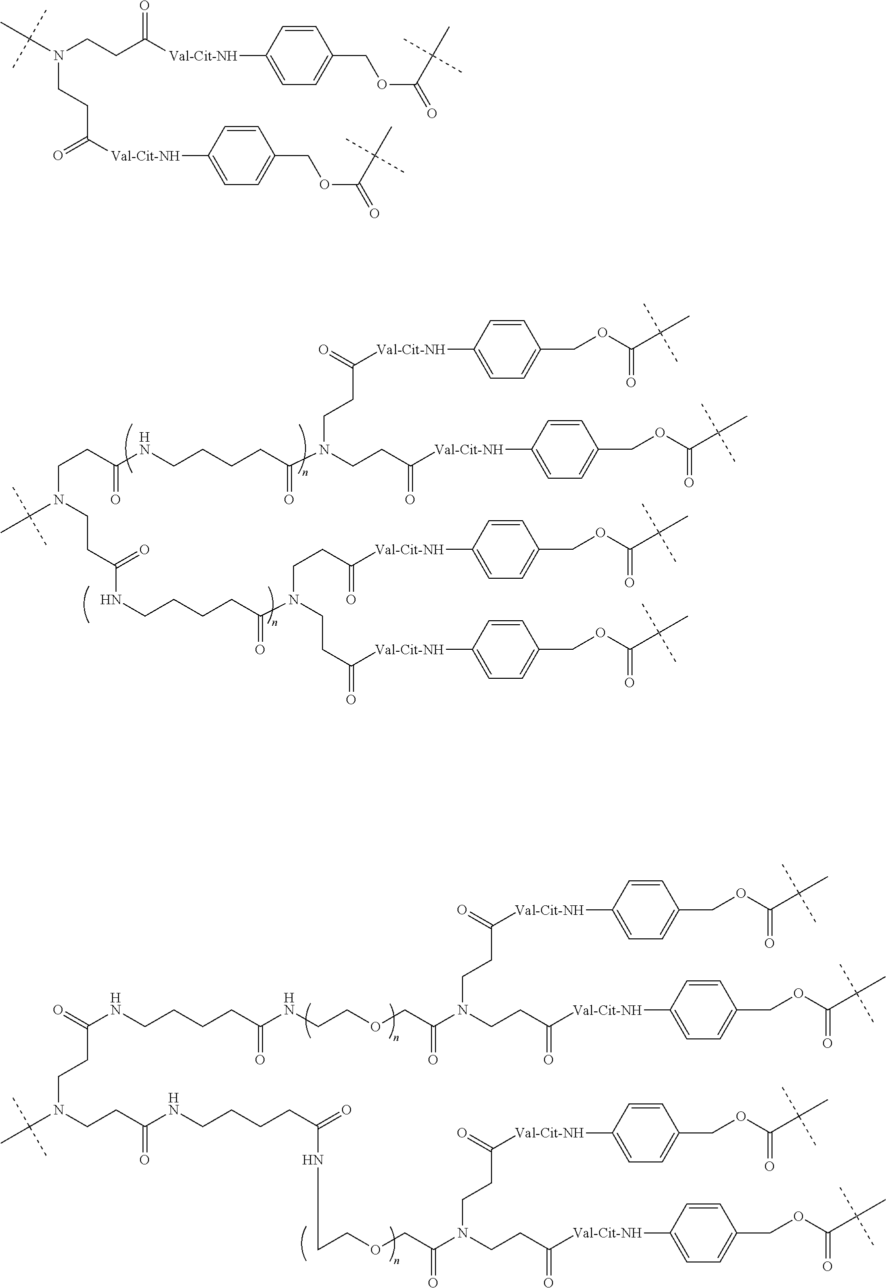

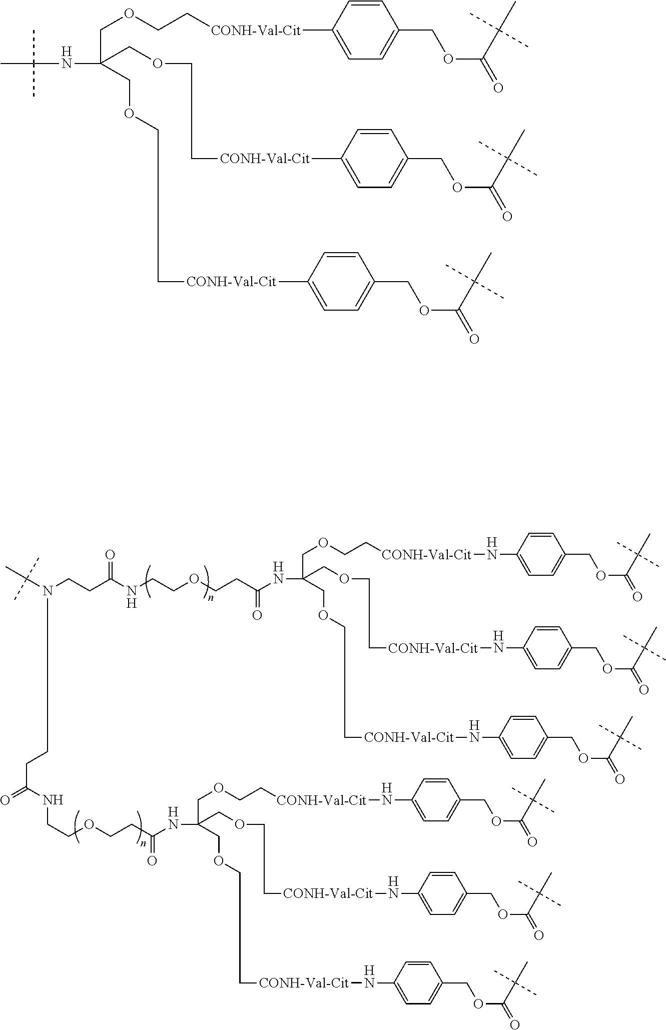

Wherein n=is an integer selected from 1 to 10.

### VI. Linkage Group

Different moieties of the conjugates of the present invention can be connected via various chemical linkages. Examples include but are not limited to amide, ester, disulfide, ether, amino, carbamate, hydrazine, thioether, and carbonate. For instance, the terminal hydroxyl group of a PEG moiety (P) may be activated and then coupled with lysine (T) to provide a desirable linkage point between P and T of Formula I or II. The linkage group between T and L¹ or between T and L² or between L² and B may be an amide resulting from the reaction between the amino group of a linker L² and the carboxyl group of Lysine (T) or between the carboxyl group of L¹ and the amino group of T or between the carboxyl group of L² and the amino group of B. Depending on the desirable characteristics of the conjugate, suitable linkage groups may also be incorporated between the antibody moiety (A) and the adjacent linker L¹ or between any two amino acids or between an amino acid and PABC.

In some embodiments, the linkage group between different moieties of the conjugates may be derived from coupling of a pair of functional groups which bear inherent chemical affinity or selectivity for each other. These types of coupling or ring formation allow for site-specific conjugation for the introduction of a protein or antibody moiety. Non-limiting examples of these functional groups that lead to site-specific conjugation include thiol, maleimide, 2′-pyridyldithio variant, methylsulfonyl pyrimidin, methylsulfonyl benzothiazole, vinylpyridine, ethyl P-ethynyl-N-(p-tolyl) phosphonamidate, aromatic or vinyl sulfone, bis-sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate. O-carbamoylhydroxylamine, trans-cyclooctene, tetrazine, and triarylphosphine, boronic acid, alkyne, Iodine.

### VII. Cytotoxic Compounds

In some embodiments, D can include but not limit to maytansinoid (U.S. Pat. No. 5,208,020; 5,416,064; EP 0425235), auristatin derivatives such as monomethyl auristatin E (MMAE) and F (MMAF) (U.S. Pat. Nos. 5,635,483; 5,780,588; 7,498,298), dolastatin, calicheamicin, pyrrolobenzodiazepine or its derivatives thereof (U.S. Pat. Nos. 5,712,374; 5,714,586; 5,739,116; 5,767,285; 5,770,701; 5,770,710; 5,773,001: U.S. Pat. No. 5,877,296; Hinman, L. M. el al. Cancer Res., 1993, 53, 3336-3342; Lode, H. N. et al. Cancer Res., 1998, 58, 2925-2928), anthracycline such as daunomycin or doxorubicin (Kratz, F. et al. Curr. Med. Chem., 2006, 13, 477-523; Jeffrey, S. C. et al. Bioorg. Med. Chem. Lett., 2006, 16, 358-362; Torgov, M. Y. et al. Bioconjug. Chem., 2005, 16 717-721; Nagy, A. et al. Proc. Natl. Acad. Sci. USA, 2000, 97, 829-834; Dubowchik, G. M. et al. Bioorg. Med. Chem. Lett., 2002, 12, 1529-1532; King, H. D. et al. J. Med. Chem., 2002, 45, 4336-4343; U.S. Pat. No. 6,630,579), methotrexate, vindesine, taxanes such as docetaxel, paclitaxel, larotaxel, tesetaxel, and ortataxel, trichothecene and CC-1065.

In other embodiments, D can be an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, and enomycin.

In yet some other embodiments, D can be a radioactive atom. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu. When the radioconjugate is used for detection, it may comprise a radioactive atom for scintigraphic studies, for example Tc⁹⁹ or I¹²³, or a spin label for magnetic resonance imaging (MRI), such as I¹²³ again, I¹³¹, In¹¹¹, F¹⁹, C¹³, N¹⁵, O¹⁷, gadolinium, manganese or iron.

In some more embodiments, D can include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its synthetic analogues, adozelesin, carzelesin and bizelesin); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics, e.g., calicheamicin (Nicolaou, K. C. et al. Agnew Chem. Intl. Ed., 1994, 33, 183-186), dynemicin, esperamicin, as well as neocarzinostatin chromophore and related chromoprotein enediyne antibiotic chromomophores, aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, 5 ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; epothilone, oligomycin C; PNU-15%82; valrubicin; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, e.g. paclitaxel (TAXOL®) and doxetaxel (TAXOTERE®); chlorambucil; gemcitabine; 6-thioguanine; mercaptopurine; platinum analogs such as cisplatin and carboplatin; vinblastine; platinum; etoposide (VP-16); ifosfamide; GDC-0068; mitomycin C; mitoxantrone; tubulysins (IM-2, Tubulysin B); vincristine; vinorelbine; navelbine; novantrone; teniposide; daunomycin; aminopterin; xeloda; ibandronate; CPT-11; topoisomerase inhibitor rubitecan (9-nitrocamptothecin or RFS-2000); difluoromethylomithine; retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, ralo xifene, 4-hydroxytamoxifen, trioxifene, keoxifene, LY117018, onapristone, and toremifene; and anti-androgens such as flutamide, nilutamide, bicalutamide, leuprolide, and goserelin; and pharmaceutically acceptable salts, acids or derivatives of any of the above.

### VIII. Bispecific Antibody

In some embodiment, a BsADC comprises a bispecific single chain antibody, wherein the two binding domains of the bispecific single chain antibody are linked via a peptide linker or other chemical linkers. In some embodiments, the linker comprises a cysteine, azide, DBCO or an unnatural amino acid residue that can be used for site-specific conjugation of the antibody to a non-immunogenic polymer drug conjugate, e.g. PEGylated drug conjugate. In some other embodiments, one or both of the two binding domains of the bispecific single chain antibody comprises a cysteine or an unnatural amino acid residue that can be used for site-specific conjugation of the antibody to anon-immunogenic polymer drug conjugate, e.g. PEGylated drug conjugate.

In a preferred embodiment, bispecific antibody could be a single chain bispecific antibody, a bispecific nanobody or other bispecific antigen binding fragment, or combination of thereof that targets CD47 and PD-L1.

### IX. Synthesis

Once the desired size and number of branches of PEG have been selected, the terminal functional group of PEG such as hydroxyl, carboxyl group and the like can be converted to terminal branched heterobifunctional groups using any art-recognized process (WO2018075308). Broadly stated, the terminal branched heterobifunctional PEG can be prepared by activating terminal hydroxyl or carboxyl group of the PEG with N-Hydroxysuccinimide using reagents such as Di(N-succinimidyl) carbonate (DSC), triphosgene and the like in the case of terminal hydroxyl group or using coupling reagents such as N,N-Diisopropylcarbodiimide (DIPC), 1-Ethyl-3-(3-dimethylaminopropyl)carbodiimide (EDC) and the like in the case of terminal carboxyl group in the presence of base such as 4-Dimethylaminopyridine (DMAP), pyridine and the like to form activated PEG.

Next, the activated PEG can be reacted with a trifunctional small molecule such as lysine derivative H-Lys(Boc)-OH in the presence of base such as Diisopropylamine (DIPEA) to form a terminal branched heterobifunctional PEG with a free carboxyl group and a Boc-protected amino group PEG-Lys(Boc)-COOH. As will be appreciated by those of ordinary skill, other terminal functional groups of PEG such as halide, amino, thiol group and the like and other trifunctional small molecules containing any combination of three functional groups from the list of —NH₂, —NHNH₂, —COOH, —OH, —C(O)X (X=halides), —N═C═O, —SH, anhydride, halides, maleimido, C═C, C≡C and the like or their protected version can be used as alternatives for the same purpose if desired.

The terminal branched heterobifunctional PEG-Lys(Boc)-COOH can then be used to react with branched moiety such as

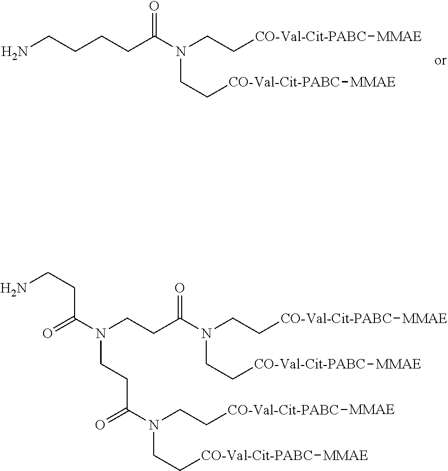

to form PEGylated drug conjugate PEG-Lys(Mal)-(Val-Cit-PABC-MMAE)_{2 or 4}.

Bispecific antibodies SCACD47/SCAPD-L1 can be prepared through genetic manipulation of expression systems. DNA encoding a bispecific single chain fragments of SCACD47 and SCAPD-L1 can be synthesized and introduced into an expression system (e.g, CHO cells). The protein of interest is then expressed and purified through chromatography technologies.

To prepare a PEGylated single chain BsADC, the PEGylated drug conjugate with functional group maleimide or DBCO can be reacted site specifically with free thiol or azide functional group of a bispecific antibody SCACD47/SCAPD-L1 that is either genetically inserted or through derivatization, to form PEG-Lys(SCACD47/SCAPD-L1)-(Val-Cit-PABC-MMAE)_{2 or 4}.

In addition to thiol/maleimide or DBCO/azide site-specific conjugation group pair exemplified in this invention, as will be appreciated by those of ordinary skill, other known pairs of site-specific conjugation groups, such as thiol/methylsulfonyl pyrimidin pair; thiol/methylsulfonyl benzothiazole pair; thiol/ethyl P-ethynyl-N-(p-tolyl) phosphonamidate pair; thio/vinylpyridine pair; trans-cyclooctenes/tetrazines pair; carbonyl/hydrazide; carbonyl/oxime; Suzuki-Miyaura Cross-Coupling reagent pair; Sonogashira Cross-Coupling reagent pair; Staudinger Ligation reagent pair; Knoevenagel-Intra Michael addition reagent pair and the like can be similarly designed and used as alternatives for the same purpose if desired. The foregoing list of site-specific conjugation group pairs is merely illustrative and not intended to restrict the type of site-specific conjugation group pairs suitable for use herein.

### X. Compositions

The present invention also provides a composition, e.g., a pharmaceutical composition, containing the compound of the present invention, formulated together with a pharmaceutically acceptable carrier. For example, a pharmaceutical composition of the invention can comprise a compound (e.g. a bispecific antibody-drug conjugate) that binds to two different receptors CD47 and PD-L1.

Therapeutic formulations of this invention can be prepared by mixing the bispecific molecule drug conjugate having the desired degree of purity with optional physiologically acceptable carriers, excipients or stabilizers in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 amino acid residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween, Pluronics, or PEG.

The formulation may also contain more than one active compound as necessary for the particular indication being treated, preferably those with complementary activities that do not adversely affect each other. For instance, the formulation may further comprise another antibody or bispecific antibody, cytotoxic agent, chemotherapeutic agent or ADC. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions of the invention can be administered in combination therapy, i.e., combined with other agents. Examples of therapeutic agents that can be used in combination therapy are described in greater detail below.

The formulations to be used for in vivo administration must be sterile. This can be readily accomplished by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

### XI. Dosage

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the bispecific molecule drug conjugate of this invention, the dosage ranges from about 0.0001 to 100 mg/kg, and more usually 0.01 to 50 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration daily, twice per week, once per week, once every two weeks, once every three weeks, once every four weeks, once a month, once every 3 months or once every three to 6 months. Preferred dosage regimens for bispecific drug conjugate of the invention include 1 mg/kg body weight or 3 mg/kg body weight via intravenous administration, with the bispecific drug conjugate being given using one of the following dosing schedules: (i) every four weeks for six dosages, then every three months; (ii) every three weeks; (iii) 3 mg/kg body weight once followed by 1 mg/kg body weight every three weeks.

Alternatively, bispecific drug conjugate can be administered as a sustained release formulation, in which case less frequent administration is required. Dosage and frequency vary depending on the half-life of the bispecific drug conjugate in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a bispecific molecule of the invention preferably results in a decrease in severity of disease symptoms, an increase in frequency and duration of disease symptom-free periods, or a prevention of impairment or disability due to the disease affliction. For example, for the treatment of tumors, a “therapeutically effective dosage” preferably inhibits cell growth or tumor growth or metastasis by at least about 20%, more preferably by at least about 40%, even more preferably by at least about 60%, and still more preferably by at least about 80% relative to untreated subjects. The ability of an agent or compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, metastasis, or otherwise ameliorate symptoms in a subject. One of ordinary skill in the art would be able to determine such amounts based on such factors as the subject's size, the severity of the subject's symptoms, and the particular composition or route of administration selected.

### XII. Administration

A composition of the invention can be administered via one or more routes of administration using one or more of a variety of methods known in the art. As will be appreciated by the skilled artisan, the route and/or mode of administration will vary depending upon the desired results. Preferred routes of administration for antibody drug conjugate of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a bispecific molecule drug conjugate of the invention can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker. Inc., New York, 1978.

Therapeutic compositions can be administered with medical devices known in the art. For example, a therapeutic composition of the invention can be administered with a needleless hypodermic injection device, such as the devices disclosed in U.S. Pat. Nos. 5,399,163, 5,383,851, 5,312,335, 5,064,413. U.S. Pat. Nos. 4,941,880, 4,790,824, and 4,596,556. Examples of well-known implants and modules useful in the present invention include those described in U.S. Pat. Nos. 4,487,603, 4,486,194, 4,447,233, 4,447,224, 4,439,196, and 4,475,196. These patents are incorporated herein by reference. Many other such implants, delivery systems, and modules are known to those skilled in the art.

### XIII. Treatment Methods

In one aspect, the present invention relates to treatment of a subject in vivo using the above-described bispecific molecule drug conjugate such that growth and/or metastasis of cancerous tumors is inhibited. In one embodiment, the invention provides a method of inhibiting growth and/or restricting metastatic spread of tumor cells in a subject, comprising administering to the subject a therapeutically effective amount of a bispecific molecule drug conjugate.

Non-limiting examples of preferred cancers for treatment include chronic or acute leukemia including acute myeloid leukemia, chronic myeloid leukemia, acute lymphoblastic leukemia, chronic lymphocytic leukemia, lymphocytic lymphoma, breast cancer, ovarian cancer, melanoma (e.g., metastatic malignant melanoma), renal cancer (e.g. clear cell carcinoma), prostate cancer (e.g. hormone refractory prostate adenocarcinoma), colon cancer and lung cancer (e.g. non-small cell lung cancer). Additionally, the invention includes refractory or recurrent malignancies whose growth may be inhibited using the antibodies of the invention. Examples of other cancers that may be treated using the methods of the invention include bone cancer, pancreatic cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

As used herein, the term “subject” is intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of enhancement of an immune response. The methods are particularly suitable for treating human patients having a disorder that can be treated by augmenting the immune response.

The above treatment may also be combined with standard cancer treatments. For example, it may be effectively combined with chemotherapeutic regimes. In these instances, it may be possible to reduce the dose of chemotherapeutic reagent administered (Mokyr, M. et al. Cancer Res., 1998, 58, 5301-5304).

Other antibodies which may be used to activate host immune responsiveness can be used in or with the bispecific molecule drug conjugate of this invention. These include molecules targeting on the surface of dendritic cells which activate DC function and antigen presentation. For example, anti-CD40 antibodies are able to substitute effectively for T cell helper activity (Ridge, J. et al. Nature, 1998, 393, 474-478) and can be used in conjunction with the bispecific molecule drug conjugate of this invention (Ito, N. et al. Immunobiology, 2000, 201, 527-540). Similarly, antibodies targeting T cell costimulatory molecules such as CTLA-4 (U.S. Pat. No. 5,811,097), CD28 (Haan, J. et al. Immunol. Lett., 2014, 162, 103-112), OX-40 (Weinberg, A. et al. J. Immunol., 2000, 164, 2160-2169), 4-1BB (Melero, I. et al. Nature Med., 1997, 3, 682-685), and ICOS (Hutloff, A. et al. Nature, 1999, 397, 262-266) or antibodies targeting PD-1 (U.S. Pat. No. 8,008,449) and PD-L1 (U.S. Pat. No. 7,943,743; 8,168,179) may also provide for increased levels of T cell activation. In another example, the bispecific molecule drug conjugate of this invention can be used in conjunction with anti-neoplastic antibodies, such as RITUXAN (rituximab), HERCEPTIN (trastuzumab), BEXXAR (tositumomab), ZEVALIN (ibritumomab), CAMPATH (alemtuzumab), LYMPHOCIDE (epratuzumab), AVASTIN (bevacizumab), and TARCEVA (erlotinib), and the like.

### Definitions of Terms

The term “alkyl” as used herein refers to a hydrocarbon chain, typically ranging from about 1 to 25 atoms in length. Such hydrocarbon chains are preferably but not necessarily saturated and may be branched or straight chain, although typically straight chain is preferred. The term C₁₋₁₀ alkyl includes alkyl groups with 1, 2, 3, 4, 5, 6, 7, 8, 9 and 10 carbons. Similarly C₁₋₂₅ alkyl includes all alkyls with 1 to 25 carbons. Exemplary alkyl groups include methyl, ethyl, isopropyl, n-butyl, n-pentyl, 2-methyl-1-butyl, 3-pentyl, 3-methyl-3-pentyl, and the like. As used herein, “alkyl” includes cycloalkyl when three or more carbon atoms are referenced. Unless otherwise noted, an alkyl can be substituted or unsubstituted.

The term “functional group” as used herein refers to a group that may be used, under normal conditions of organic synthesis, to form a covalent linkage between the entity to which it is attached and another entity, which typically bears a further functional group. A “bifunctional linker” refers to a linker with two functional groups forms two linkages via with other moieties of a conjugate.

The term “derivative” as used herein refers to a chemically-modified compound with an additional structural moiety for the purpose of introducing new functional group or tuning the properties of the original compound.

The term “protecting group” as used herein refers to a moiety that prevents or blocks reaction of a particular chemically reactive functional group in a molecule under certain reaction conditions. Various protecting groups are well-known in the art and are described, for example, in T. W. Greene and G. M. Wuts, Protecting Groups in Organic Synthesis. Third Edition, Wiley, New York, 1999, and in P. J. Kocienski, Protecting Groups, Third Ed., Thieme Chemistry, 2003, and references cited therein.

The term “PEG” as used herein refers to polyethylene glycol. PEGs for use in the present invention typically comprise a structure of —(CH₂CH₂O)ₙ—. PEGs may have a variety of molecular weights, structures or geometries. A PEG group may comprise a capping group that does not readily undergo chemical transformation under typical synthetic reaction conditions. Examples of capping groups include —OC₁₋₂₅ alkyl or —OAryl.

The term “linker” as used herein refers to an atom or a collection of atoms used to link interconnecting moieties, such as an antibody and a polymer moiety. A linker can be cleavable or noncleavable. The preparation of various linkers for conjugates have been described in literatures including for example Goldmacher et al., Antibody-drug Conjugates and Immunotoxins: From Pre-clinical Development to Therapeutic Applications, Chapter 7, in Linker Technology and Impact of Linker Design on ADC properties, Edited by Phillips G L; Ed. Springer Science and Business Media, New York (2013). Cleavable linkers incorporate groups or moieties that can be cleaved under certain biological or chemical conditions. Examples include enzymatically cleavable disulfide linkers, 1,4- or 1,6-benzyl elimination, trimethyl lock system, bicine-based self-cleavable system, acid-labile silyl ether linkers and other photo-labile linkers.

The term “linking group” or “linkage group” as used herein refers to a functional group or moiety connecting different moieties of a compound or conjugate. Examples of a linking group include, but are not limited to, amide, ester, carbamate, ether, thioether, disulfide, hydrazone, oxime, and semicarbazide, carbodiimide, acid labile group, photolabile group, peptidase labile group and esterase labile group. For example, a linker moiety and a polymer moiety may be connected to each other via an amide or carbamate linkage group.

The terms “peptide,” “polypeptide,” and “protein” are used herein interchangeably to describe the arrangement of amino acid residues in a polymer. A peptide, polypeptide, or protein can be composed of the standard 20 naturally occurring amino acid, in addition to rare amino acids and synthetic amino acid analogs. They can be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques, i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

An “isolated” peptide, polypeptide, or protein refers to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein described in the invention can be purified from a natural source, produced by recombinant DNA techniques, or by chemical methods.

An “antigen” refers to a substance that elicits an immunological reaction or binds to the products of that reaction. The term “epitope” refers to the region of an antigen to which an antibody or T cell binds.

The term “antibody” as referred to herein includes whole antibodies and any antigen binding fragment or single chains thereof. Whole antibodies are glycoproteins comprising at least two heavy (H) chains and two light (L) chains inter-connected by disulfide bonds. Each heavy chain is comprised of a heavy chain variable region (V_{H}) and a heavy chain constant region. The heavy chain constant region is comprised of three domains, C_{H}1, C_{H}2 and C_{H}3. Each light chain is comprised of a light chain variable region (V_{L}) and a light chain constant region (C_{L}), the light chain constant region is comprised of one domain. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

As used herein, “antibody fragments”, may comprise a portion of an intact antibody, generally including the antigen binding and/or variable region of the intact antibody. Examples of antibody fragments include linear antibodies; single-chain antibody molecules; and multispecific antibodies formed from antibody fragments.

The term “antigen-binding fragment or portion” of an antibody (or simply “antibody fragment or portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)₂ fragment, a bivalent fragment comprising two Fab fragments linked by a disulfide bridge at the hinge region; (iii) a Fab′ fragment, which is essentially an Fab with part of the hinge region; (iv) a Fd fragment consisting of the V_{H} and C_{H}I domains; (v) a Fv fragment consisting of the V_{L} and V_{H} domains of a single arm of an antibody, (vi) a dAb, which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. Science 1988, 242, 423-426; and Huston et al. Proc. Natl. Acad Sci. USA 1988, 85, 5879-5883. Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

As used herein, the term “Fc fragment” or “Fc region” is used to define a C-terminal region of an immunoglobulin heavy chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein (Kohler, G. et al. Nature, 1975, 256, 495-497), which is incorporated herein by reference, or may be made by recombinant DNA methods (U.S. Pat. No. 4,816,567), which is incorporated herein by reference. The monoclonal antibodies may also be isolated from phage antibody libraries using the techniques described by Clackson et al., Nature, 1991, 352, 624-628 and Marks et al., J Mol Biol, 1991, 222, 581-597, for example, each of which is incorporated herein by reference.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; Morrison et al., Proc Natl Acad Sci USA, 1984, 81, 6851-6855; Neuberger et al., Nature, 312, 1984, 604-608; Takeda et al., Nature, 1985, 314, 452-454; International Patent Application No. PCT/GB85/00392, each of which is incorporated herein by reference).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody optionally also will comprise at least a portion of an immunoglobulin constant region (Fc), typically that of a human immunoglobulin. For further details, see Jones et al., Nature, 1986, 321, 522-525; Riechmann et al., Nature, 1988, 332, 323-329; Presta, Curr Op Struct Biol, 1992, 2, 593-596; U.S. Pat. No. 5,225,539, each of which is incorporated herein by reference.

“Human antibodies” refer to any antibody with fully human sequences, such as might be obtained from a human hybridoma, human phage display library or transgenic mouse expressing human antibody sequences.

The term “pharmaceutical composition” refers to the combination of an active agent with a carrier, inert or active, making the composition especially suitable for diagnostic or therapeutic use in vivo or ex vivo.

As used herein, “pharmaceutically acceptable carrier” includes any and all solvents, dispersion media, coatings, antibacterial and antifungal agents, isotonic and absorption delaying agents, and the like that are physiologically compatible. A “pharmaceutically acceptable carrier”, after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see e.g., Berge, S. M., el al. J. Pharm. Sci. 1997, 66, 1-19).

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat a neoplastic condition is an amount that will cause, for example, a reduction in tumor size, a reduction in the number of tumor foci, or slow the growth of a tumor, as compared to untreated animals.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### EXAMPLES

The following examples serve to provide further appreciation of the invention but are not meant by any way to restrict the effective scope of the invention.

### Example 1. Preparation of Intermediates

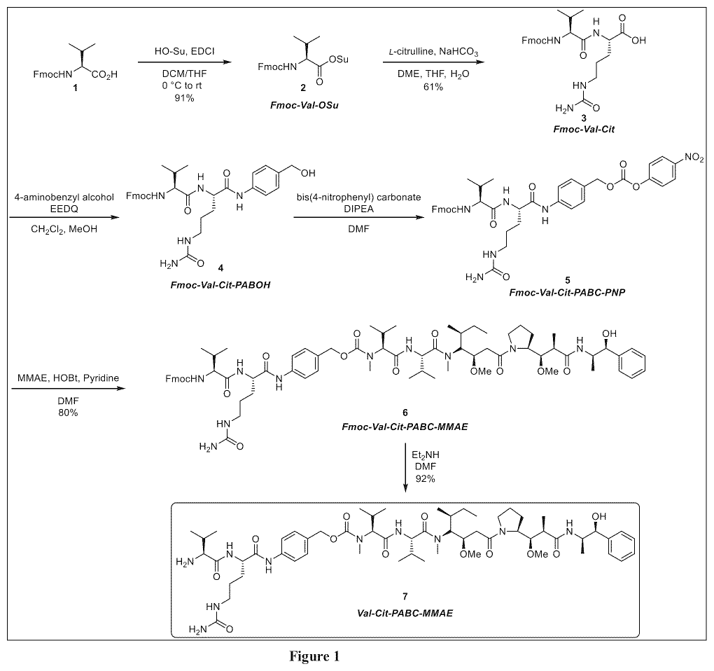

Preparation of Val-Cit-PABC-MMAE (compound 7, FIG. 1):
Fmoc-Val-O Su (2): Fmoc-Val-OH (20.3 g, 60.0 mmol) and N-hydroxysuccinimide (9.0 g, 78.0 mmol) were dissolved in a mixture of CH₂Cl₂ (120 mL) and THF (40 mL). Separately. EDCI (13.8 g, 72.0 mmol) was solubilized in CH₂Cl₂ (200 mL) and the solution was cooled to 0-5° C. The Fmoc-Val-OH/NHS solution was then added to the EDCI solution before warming up the reaction mixture to room temperature. The reaction mixture was stirred at room temperature until reaction was complete. The reaction mixture was then concentrated under reduced pressure and azeotropically distilled twice with THF (100 mL). The concentrated residue was dissolved with THF (800 mL) and filtered to remove EDU. The filtrate was concentrated under reduced pressure and re-slurried with n-heptane (800 mL) at 5-10° C. for 12 h. Solids were filtered, washed and dried under vacuum to yield Fmoc-Val-OSu (2) (23.8 g, 91%) as white powder. HRMS (EST) calcd for C₂₄H₂₄N₂O₆Na [M+Na]⁺ 459.1532, found 459.1523.
Fmoc-Val-Cit (3): Fmoc-Val-OSu (9.8 g, 22.5 mmol) was dissolved in DME (150 mL) at room temperature. Separately, sodium bicarbonate (2.1 g, 24.7 mmol) was solubilized in water (150 mL) at room temperature and L-citrulline (4.3 g, 24.7 mmol) was then added to give a homogeneous clear solution. The prepared L-citrulline solution was then added to the Fmoc-Val-OSu solution, then THF (75 mL) was added to the solution and the reaction mixture was stirred at room temperature for 16 h until reaction was complete. The reaction mixture was acidified with 15% citric acid (200 mL), then concentrated in vacuum. The mixture was suspended in water (500 mL) and the resulting mixture was stirred for 2 h before filtered and dried in vacuum. The solids were re-suspended in methyl tert-butyl ether (500 mL) and stirred for 12 h before being filtered, washed and dried under vacuum to yield Fmoc-Val-Cit (3) (6.8 g, 61%) as white powder. HRMS (ESI) calcd for C26H33N4O6 [M+H]+497.2400, found 497.2388.
Fmoc-Val-Cit-PABOH (4): A solution of compound 3 (4.96 g, 10.0 mmol) and 4-aminobenzyl alcohol (2.46 g, 20.0 mmol) in CH2Cl2 (350 mL) and MeOH (150 mL) was added EEDQ (4.95 g, 20.0 mmol), the reaction mixture was stirred at room temperature for 24 h. Then additional EEDQ (2.5 g, 10.0 mmol) was added and stirred for another 24 h. After the reaction was complete, the solvent was removed under reduced pressure and the resulting residue was re-slurried in methyl tert-butyl ether (800 mL) for 12 h. Solids were filtered, washed and dried under vacuum to yield the compound 4 (4.1 g, 69%) as white powder. HRMS (ESI) calcd for C₃₃H₄₀N₅O₆ [M+H]⁺ 602.2979, found 602.2969.
Fmoc-Val-Cit-PABC-PNP (5): To a solution of compound 4 (5.2 g, 8.6 mmol) and bis(4-nitrophenyl) carbonate (4.9 g, 16.1 mmol) in DMF (52 mL) at room temperature was added DIPEA (2.5 mL, 15.0 mmol). The reaction mixture was stirred at room temperature for 5 h until reaction was complete. The product was precipitated out of the reaction mixture by adding anhydrous ethyl acetate (250 mL) and methyl tert-butyl ether (250 mL). The resulting slurry was stirred, then cooled to 0° C. and stirred for 30 min. The solids were isolated by filtration, washed and dried under vacuum to yield Fmoc-Val-Cit-PABC-PNP (5) (4.7 g, 72%) as pale yellow powder. HRMS (ESI) calcd for C₄₀H₄₃N₆O₁₀ [M+H]⁺ 767.3041, found 767.3045.
Fmoc-Val-Cit-PABC-MMAE (6): Compound MMAE (2.0 g, 1.8 mmol) and Fmoc-Val-Cit-PABC-PNP (5) (2.8 g, 3.6 mmol) were solubilized in DMF (20 m L). HOBt (0.75 g, 5.6 mmol) and pyridine (1.7 mL) were then added and the reaction mixture was stirred at room temperature for 24 h until the reaction was complete. The reaction mixture was cooled to 0° C. and was added methyl tert-butyl ether (180 mL). The resultant slurry was stirred for 3-5 h and filtered, washed and dried under vacuum. The crude product was purified by column purification to yield Fmoc-Val-Cit-PABC-MMAE (6)(3.0 g, 80%) as yellow powder. HRMS (ESI) calcd for C₇₃H₁₀₅N₁₀O₁₄ [M+H]⁺ 1345.7812, found 1345.7820.

Val-Cit-PABC-MMAE (7): Compound 6 (3.0 g, 2.2 mmol) was suspended in anhydrous DMF (40 mL) and the resulting suspension was stirred at room temperature until a homogeneous suspension formed. Diethylamine (10 mL) was then added and the reaction mixture was stirred at room temperature for 3 h until reaction was complete. Methyl tert-butyl ether (100 mL) and ethyl acetate (50 mL) was then added over 60 min. The resulting mixture was stirred for 4 h at 0° C. Solids were filtered and dried under vacuum to yield Val-Cit-PABC-MMAE (7) (2.3 g, 92%) as pale yellow powder. HRMS (ESI) calcd for C₅₈H₉₅N₁₀O₁₂ [M+H]⁺ 1123.7131, found 1123.7142.

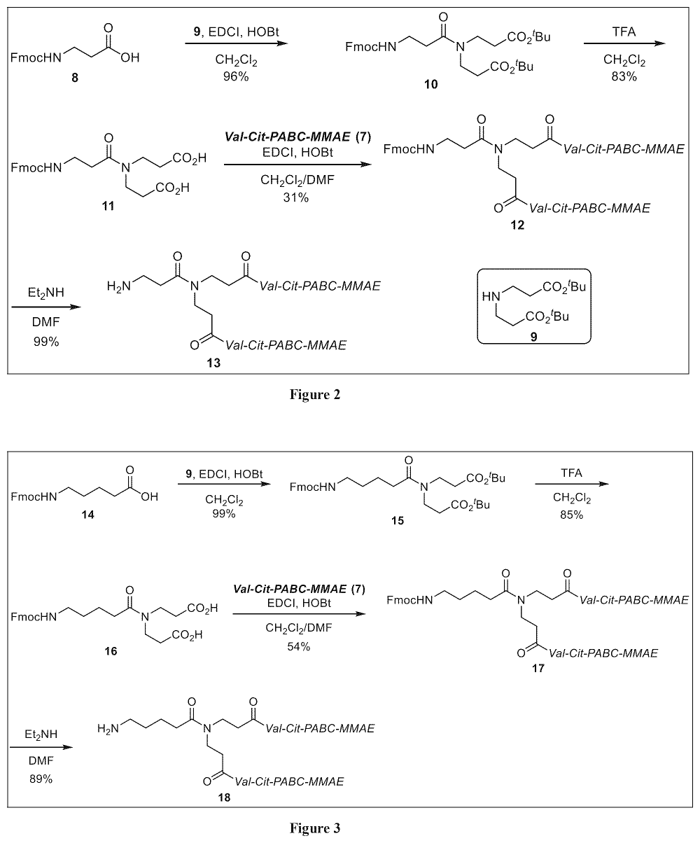

### Preparation of Branched Linker Intermediate Compound 13 (FIG. 2)

Compound 10: To a solution of compound 8 (0.62 g, 2.0 mmol) in dry CH₂Cl₂ (15 mL) at room temperature under argon, Di-tert-butyl 3,3′-azanediyldipropanoate (9) (0.62 mL, 2.2 mmol), EDCI (0.58 g, 3.0 mmol) and HOBt (54 mg, 0.4 mmol) were added. The mixture was stirred at room temperature until full conversion was observed by TLC. After the reaction was completed, the mixture was extracted with CH₂Cl₂ (30 mL×2), and the organic layer was washed with brine (20 mL), dried over Na₂SO₄, filtered and concentrated in vacuum. The crude reaction mixture was purified through chromatography on silica gel to yield the compound 10 (1.1 g, 96%) as colorless oil. HRMS (ESI) calcd for C₃₂H₄₃N₂O₇ [M+H]⁺ 567.3070, found 567.3062.
Compound 11: Compound 10 (5.2 g, 9.2 mmol) was dissolved in CH₂Cl₂ (100 mL) followed by addition of TFA (25 mL). The mixture was stirred at room temperature for 3 h. The solvent was removed under vacuum as much as possible at <35° C. The residue was purified through chromatography on silica gel to yield the compound 11 (3.4 g, 83%) as colorless oil. HRMS (ESI) calcd for C₂₄H₂₇N₂O₇ [M+H]⁺ 455.1818, found 455.1824.
Compound 12: To a stirred solution of compound 11 (41 mg, 0.091 mmol) in dry CH₂C₂ (2 mL) and DMF (2 mL) at room temperature under argon, Val-Cit-PABC-MMAE (7) (224 mg, 0.2 mmol), EDCI (52 mg, 0.27 mmol) and HOBt (5 mg, 0.04 mmol) were added. The mixture was stirred at room temperature until full conversion was observed by TLC. After the reaction was completed, the mixture was concentrated in vacuum. The crude reaction mixture was purified through preparative HPLC using Welch Ultimate XB-C18 column (eluents: A=0.1% TFA in water, B=MeCN) to yield the compound 12 (74 mg, 31%) as pale yellow solid. HRMS (ESI) calcd for C₁₄₀H₂₁₂N₂₂O₂₉ [M+2H]²⁺ 1333.2912, found 1333.2907.
Compound 13: Diethylamine (0.6 mL) was added to a solution of compound 12 (73 mg) in DMF (3 ml), then the reaction was allowed to proceed at room temperature for 4 h. The reaction mixture was concentrated in vacuum and the residue was purified through preparative HPLC using Welch Ultimate XB-C18 column (eluents: A=0.1% TFA in water, B=MeCN) to yield the compound 13 (71 mg, 99%) as pale yellow solid. HRMS (ESI) calcd for C₁₂₅H₂₀₂N₂₂O₂₇ [M+2H]²⁺ 1222.2572, found 1222.2560.

### Preparation of Branched Linker Intermediate Compound 18 (FIG. 3)

Compound 15: To a solution of compound 14 (0.68 g, 2.0 mmol) in dry CH₂Cl₂ (10 mL) at room temperature under argon, Di-tert-butyl 3,3′-azanediyldipropanoate (9)(0.64 mL, 2.2 mmol), EDCI (0.58 g, 3.0 mmol) and HOBt (54 mg, 0.4 mmol) were added. The mixture was stirred at room temperature until full conversion was observed by TLC. After the reaction was completed, the mixture was extracted with CH₂Cl₂ (30 mL×2), and the organic layer was washed with brine (20 mL), dried over Na₂SO₄, filtered and concentrated in vacuum. The crude reaction mixture was purified through chromatography on silica gel to yield the compound 15 (1.2 g, 99%) as colorless oil. HRMS (ESI) calcd for C₃₄H₄₇N₂O₇ [M+H]⁺ 595.3383, found 595.3380.
Compound 16: Compound 15 (0.5 g, 0.84 mmol) was dissolved in CH₂Cl2 (6.0 mL) followed by addition of TFA (3.0 mL). The mixture was stirred at room temperature for 3 h. The solvent was removed under vacuum as much as possible at <35° C. The residue was purified through chromatography on silica gel to yield the compound 16 (0.34 g, 85%) as colorless oil. HRMS (ESI) calcd for C₂₆H₃₁N₂O₇ [M+H]⁺ 483.2131, found 483.2127.
Compound 17: To a solution of compound 16 (185 mg, 0.383 mmol) in a mixture of dry CH₂Cl2 (8 mL) and DMF (8 mL) at room temperature under argon, Val-Cit-PABC-MMAE (7) (947 mg, 0.843 mmol), EDCI (238 mg, 1.23 mmol) and HOBt (26 mg, 0.19 mmol) were added. The mixture was stirred at room temperature until full conversion was observed by HPLC. After the reaction was completed, the mixture was concentrated in vacuum. The crude reaction mixture was purified through preparative HPLC using Welch Ultimate XB-C18 column (eluents: A=0.1% TFA in water, B=MeCN) to yield the compound 17 (0.56 g, 54%) as pale yellow solid. HRMS (ESI) calcd for C₁₄₂H₂₁₆N₂₂O₂₉ [M+H]⁺ 2694.6137, found 2694.6146.
Compound 18: Diethylamine (2.0 mL) was added to a solution of compound 17 (0.62 g) in DMF (5 mL) and the reaction mixture was allowed to proceed at room temperature for 2 h. The reaction mixture was concentrated in vacuum and the residue was purified through preparative HPLC using Welch Ultimate XB-C18 column (eluents: A=0.1% TFA in water, B=MeCN) to yield the compound 18 (0.51 g, 89%) as pale yellow solid. HRMS (ESI) calcd for C₁₂₇H₂₀₅N₂₂O₂₇ [M+H]⁺ 2471.5378, found 2471.5369: C₁₂₇H₂₀₆N₂₂O₂₇ [M+2H]²⁺ 1236.2728, found 1236.2744.

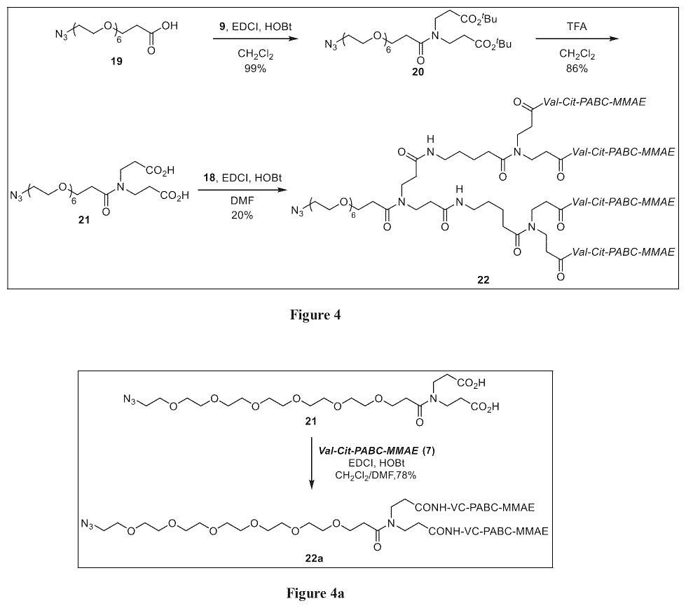

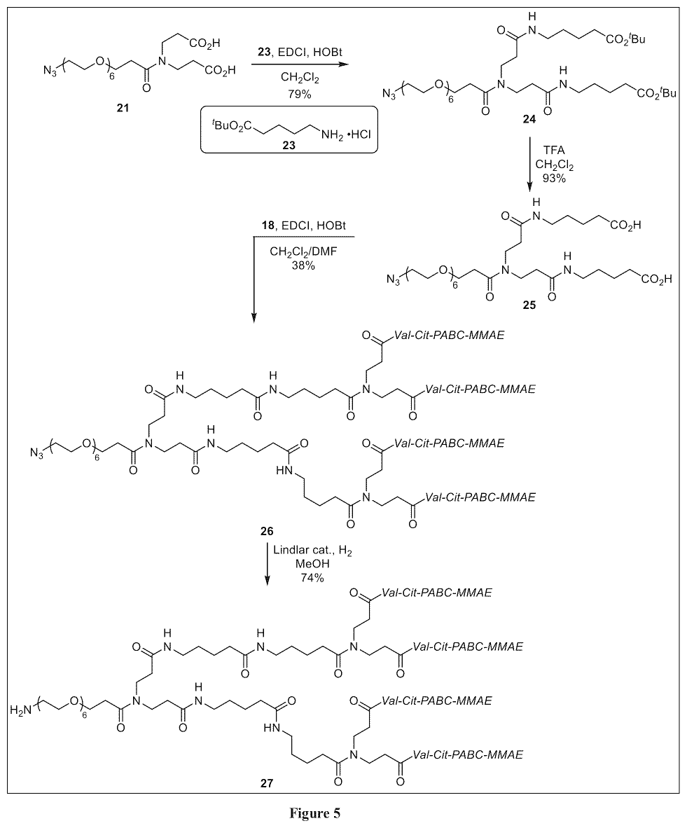

### Preparation of Branched Linker Intermediate Compound 22 (FIG. 4)

Compound 20: To a solution of compound 19 (0.76 g, 2.0 mmol) in dry CH₂Cl₂ (10 mL) at room temperature under argon, Di-tert-butyl 3,3′-azanediyldipropanoate (9) (0.64 mL, 2.2 mmol), EDCI (0.58 g, 3.0 mmol) and HOBt (54 mg, 0.4 mmol) were added. The mixture was stirred at room temperature until full conversion was observed by TLC. After the reaction was completed, the mixture was extracted with CH₂Cl₂ (30 mL×2), and the organic layer was washed with brine (20 mL), dried over Na₂SO₄, filtered and concentrated in vacuum. The crude reaction mixture was purified through chromatography on silica gel to yield the compound 20 (1.2 g, 99%) as colorless oil. HRMS (ESI) calcd for C₂₉H₅₅N₄O₁₁ [M+H]⁺ 635.3867. found 635.3860.
Compound 21: Compound 20 (0.3 g, 0.47 mmol) was dissolved in CH₂Cl₂ (4.0 mL) followed by addition of TFA (2.0 mL). The mixture was stirred at room temperature for 3 h. The solvent was removed under vacuum as much as possible at <35° C. The residue was purified through chromatography on silica gel to yield the compound 21 (0.34 g, 85%) as colorless oil. HRMS (ESI) calcd for C₂₁H₃₉N₄O₁₁ [M+H]⁺ 523.2615, found 523.2607.
Compound 22: To a stirred solution of compound 21 (39 mg, 0.076 mmol) in a mixture of dry CH₂Cl₂ (2 mL) and DMF (2 mL) at room temperature under argon, compound 18 (0.41 g, 0.17 mmol), EDCI (43 mg, 0.23 mmol) and HOBt (4.0 mg, 0.03 mmol) were added. The mixture was stirred at room temperature until full conversion was observed by HPLC. After the reaction was completed, the mixture was concentrated in vacuum. The crude reaction mixture was purified through preparative HPLC using Welch Ultimate XB-C18 column (eluents: A=0.1% TFA in water, B=MeCN) to yield the compound 22 (81 mg, 20%) as pale yellow solid. HRMS (ESI) calcd for C₂₇₅H₄₄₅N₄₈O₆₃ [M+3H]³⁺ 1810.1053, found 1810.1061, C₂₇₅H₄₄₆N₄₈O₆₃ [M+4H]⁴⁺ 1357.8310, found 1357.8346.

### Preparation of Branched Linker Intermediate Compound 22a (FIG. 4a)

Compound 22a: To a stirred solution of compound 21 (0.6 g, 1.15 mmol) in dry DMF (20 mL) at room temperature under argon, Val-Cit-PAB-MMAE (7) (2.8 g, 2.5 mmol), EDCI (0.66 g, 3.5 mmol) and HOBt (90 mg, 0.7 mmol) were added. The mixture was stirred at room temperature until full conversion was confirmed by HPLC. After the reaction was completed, the mixture was concentrated under vacuum. The residue was cooled to 0° C., followed by slow addition of methyl tert-butyl ether (100 mL). The resultant slurry was stirred for 1 h, filtered, washed and dried under vacuum. The crude product was purified on silica gel column to yield the compound 22a (2.4 g, 78%) as white solid. MS (ESI) m/z [M+2H]²⁺ 1366.94, [M+2Na]²⁺ 1389.02.

### Preparation of Branched Linker Intermediate Compound 27 (FIG. 5)

Compound 24: To a solution of compound 21 (0.57 g, 1.1 mmol) in dry CH₂Cl₂ (10 mL) at room temperature under argon, compound 23 (0.51 g, 2.4 mmol), EDCI (0.67 g, 3.5 mmol), HOBt (74 mg, 0.55 mmol) and DIPEA (0.78 mL, 4.4 mmol) were added. The mixture was stirred at room temperature until full conversion was observed by TLC. After the reaction was completed, the mixture was extracted with CH₂Cl₂ (30 mL×2), and the organic layer was washed with brine (20 mL), dried over Na₂SO₄, filtered and concentrated in vacuum. The crude reaction mixture was purified through chromatography on silica gel to yield the compound 24 (0.7 g, 79%) as colorless oil. HRMS (ESI) calcd for C₃₉H₇₃N₆O₁₃ [M+H]⁺ 833.5236, found 833.5231.
Compound 25: Compound 24 (0.52 g, 0.62 mmol) was dissolved in CH₂Cl₂ (5.0 mL) followed by addition of TFA (2.0 mL). The mixture was stirred at room temperature for 3 h. The solvent was removed under vacuum as much as possible at <35° C. The residue was purified through chromatography on silica gel to yield the compound 25 (0.42 g, 93%) as colorless oil. HRMS (ESI) calcd for C₃₁H₅₇N₆O₁₃ [M+H]⁺ 721.3984, found 721.3997.
Compound 26: To a solution of compound 25 (77 mg, 0.11 mmol) in DMF (5 mL) at room temperature under argon, compound 18 (0.58 g, 0.24 mmol), EDCI (82 mg, 0.43 mmol) and HOBt (14 mg, 0.11 mmol) were added. The mixture was stirred at room temperature until full conversion was observed by HPLC. After the reaction was completed, the mixture was concentrated in vacuum. The crude reaction mixture was purified through preparative HPLC using Welch Ultimate XB-C18 column (eluents: A=0.1% TFA in water, B=MeCN) to yield the compound 26 (0.23 g, 38%) as pale yellow solid. HRMS (ESI) calcd for C₂₈₅H₄₆₃N₅₀O₆₅[M+3H]³⁺ 1876.4854, found 1876.4851; C₂₈₅H₄₆₄N₅₀O₆₅ [M+4H]⁴⁺ 1407.6160, found 1407.6158.
Compound 27: Lindlar catalyst (130 mg, 5% by wt.) was added to a stirred solution of azide 26 (180 mg, 0.03 mmol) in methanol (10 mL). The reaction flask was evacuated and flushed with hydrogen gas. The resultant mixture was stirred under hydrogen atmosphere (balloon) at room temperature for 5 h. After completion of the reaction, the catalyst was filtered through a pad of Celite, the filter cake was washed with methanol (10 mL) and the filtrate was concentrated under reduced pressure. The crude reaction mixture was purified through preparative HPLC using Welch Ultimate XB-C18 column (eluents: A=0.1% TFA in water, B=MeCN) to yield the compound 27 (130 mg, 74%) as pale yellow solid. HRMS (ESI) calcd for C₂₈₅H₄₆₅N₄₈O₆₅ [M+3H]³⁺ 1867.8219. found 1867.8217: C₂₈₅H₄₆₆N₄₈O₆₅ [M+4H]⁴⁺ 1401.1184, found 1401.1181.

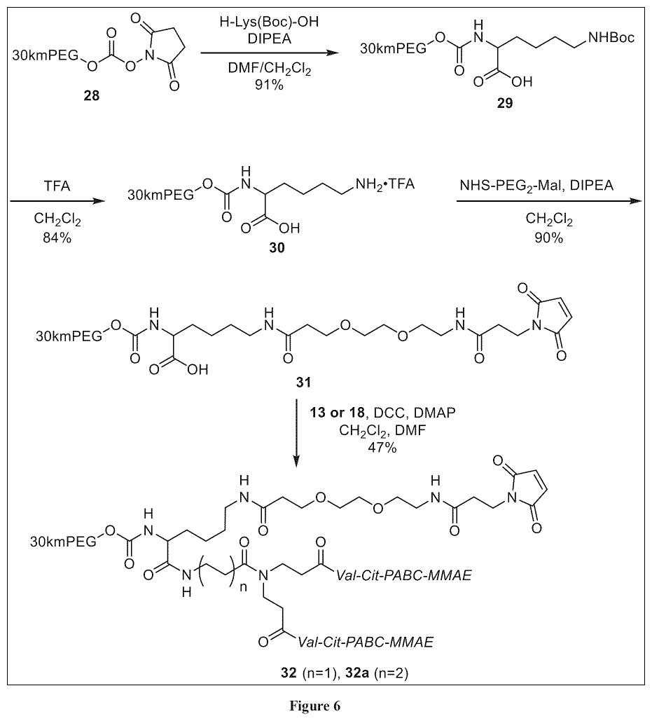

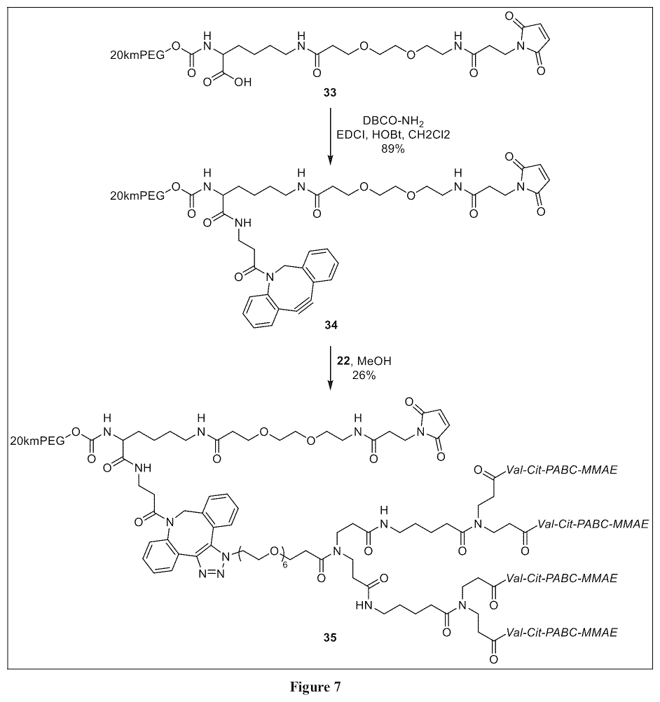

### Example 1a. Preparation of 30kmPEG-Lys(Mal)-(MMAE)₂ (Compound 32 & 32a, FIG. 6)

Compound 29: H-Lys(boc)-OH (369 mg, 1.5 mmol) was added into anhydrous DMF (100 mL) followed by addition of DIPEA (0.83 mL, 5.0 mmol), compound 28 (15 g, 0.5 mmol) and anhydrous CH₂Cl₂ (150 mL). The mixture was stirred under argon at room temperature overnight. The insoluble materials were filtered off. The solvent was removed and the residue was recrystallized from CH₂Cl₂/methyl tert-butyl ether (45 mL/300 mL). The isolated solids were recrystallized again from MeCN/2-propanol (30 mL/450 mL). The product was dried at 40° C. over 4 h under vacuum to give the compound 29 (13.6 g, 91%) as white powder.
Compound 30: A solution of compound 29 (5.7 g, 0.19 mmol) in anhydrous CH₂Cl₂ (57 mL) was added TFA (29.5 mL). The mixture was stirred at room temperature for 1 h. Solvent was removed under vacuum as much as possible at <35° C. The residue was recrystallized from CH₂Cl₂/methyl tert-butyl ether (14.5 mL/115 mL) twice. The isolated product was dried under vacuum at 40° C. to yield the compound 30 (4.7 g, 84%) as white powder.
Compound 31: To a stirred solution of compound 30 (5.5 g, 0.18 mmol) in anhydrous CH₂Cl₂ (55 mL) at 0° C. was added DIPEA (473 mg, 3.6 mmol), followed by addition of NHS-PEG₂-Mal (0.2 g, 0.47 mmol). The mixture was stirred at 0° C. for 1.5 h, then the solution was allowed to warm up slowly from 0° C. to room temperature and then stirred under argon atmosphere overnight. Solvent was removed and the residue was recrystallized from CH₂Cl₂/methyl tert-butyl ether (13.8 mL/110 mL). The isolated solids were recrystallized again from MeCN/2-propanol (11 mL/165 mL). The residue was dried under vacuum to yield the compound 31 (5.0 g, 90%) as white powder.
Compound 32: To a stirred solution of compound 31 (0.76 g, 0.025 mmol) in a mixture of DMF/CH₂Cl₂ (5 mL/5 mL) at room temperature under argon, compound 13 (0.12 g, 0.05 mmol), DCC (31 mg, 0.15 mmol) and DMAP (28 mg, 0.23 mmol) were added. The mixture was stirred at room temperature until full conversion was observed by HPLC. After the reaction was completed, the mixture was concentrated in vacuum. The crude reaction mixture was purified through preparative HPLC using Phenomenex Jupiter® C18 column (eluents: A=0.1% TFA in water, B=MeCN) to yield the compound 32 (0.36 g, 47%) as white solid.
Compound 32a: The compound 32a was prepared similarly as compound 32 by using compound 18 instead of compound 13.

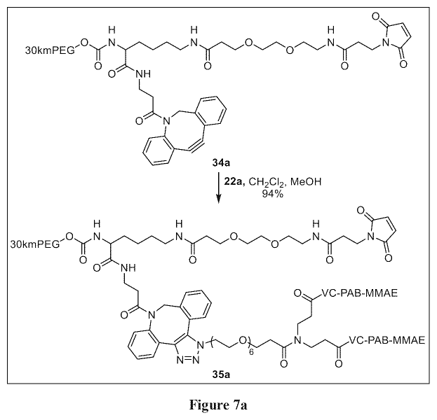

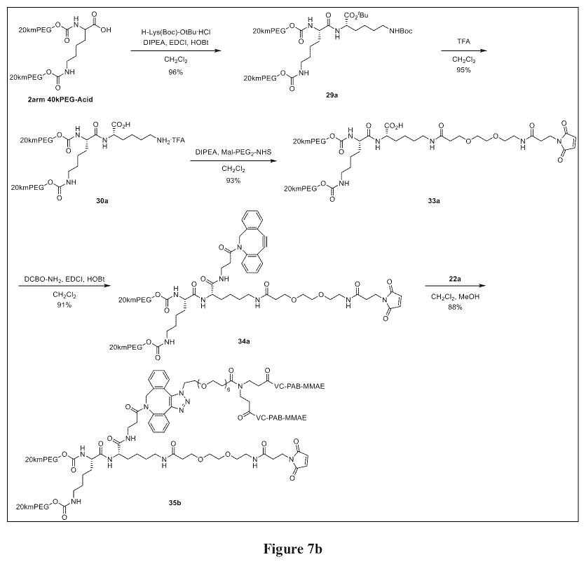

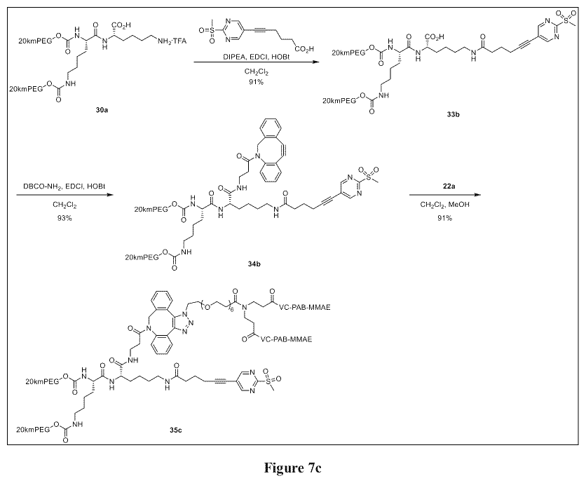

### Example 1b. Preparation of 20k mPEG-Lys(Mal)-(MMAE)₄ (Compound 35, FIG. 7)

Compound 34: To a stirred solution of compound 33 (2.0 g, 0.1 mmol) (synthesis of compound 33 refers to the procedures for preparation of compound 31) in anhydrous CH₂Cl₂ (20 mL) at room temperature under argon, DBCO-NH₂ (83 mg, 0.3 mmol), EDCI (115 mg, 0.6 mmol) and HOBt (122 mg, 0.9 mmol) were added. The mixture was stirred at room temperature until full conversion was observed by HPLC. The solvent was removed and the residue was recrystallized from CH₂Cl₂/methyl tert-butyl ether (5 mL/40 mL). The isolated solids were recrystallized again from MeCN/2-propanol (4 mL/60 mL). The product was dried at 40° C. over 4 h under vacuum to give the compound 34 (1.9 g, 89%) as white powder.
Compound 35: Compound 34 (147 mg, 0.007 mmol) was dissolved in anhydrous MeOH (3 mL), followed by addition of compound 22 (40 mg, 0.007 mmol). The mixture was stirred at room temperature for 24 h. The mixture was concentrated in vacuum and the crude reaction mixture was purified through preparative HPLC using Phenomenex Jupiter® C18 column (eluents: A=0.1% TFA in water, B=MeCN) to yield the compound 35 (41 mg, 22%) as white solid.

### Example 1c. Preparation of 30kmPEG-Lys(Mal)DBCO-(MMAE)₂ (Compound 35a, FIG. 7a)

Compound 34a was made in the similar way as the compound 34 except using 30kmPEG instead of 20kmPEG.
Compound 35a: To a stirred solution of compound 34a (12.0 g, 0.4 mmol) in anhydrous CH₂Cl₂ (60 mL) and MeOH (60 mL), compound 22a (1.31 g, 0.48 mmol) was added. The mixture was stirred at room temperature overnight until full conversion was observed by HPLC. Solvent was removed and the residue was recrystallized from MeCN/2-propanol (30 mL/450 mL) twice. The residue was dried under vacuum to yield the compound 35a (11.2 g, 94%) as white powder.

### Example 1d. Preparation of 2Arm-40kmPEG-Lys(Mal)DBCO-(MMAE)₂ (Compound 35b, FIG. 7b)

Compound 29a: To a stirred solution of 2arm-40kmPEG-CO₂H (4.0 g, 0.1 mmol) in anhydrous CH₂Cl₂ (40 mL), compound H-Lys(boc)-OtBu-HCl (102 mg, 0.3 mmol) was added followed by the addition of DIPEA (0.1 mL, 5.0 mmol), EDCI (115 mg, 0.6 mmol) and HOBt (122 mg, 0.9 mmol) at room temperature. The mixture was stirred under argon at room temperature over 16 h. The solvent was removed and the residue was recrystallized from CH₂Cl₂/methyl tert-butyl ether (12 mL/90 mL). The isolated solids were recrystallized again from MeCN/2-propanol (9 mL/140 mL). The product was dried at 40° C. over 4 h under vacuum to give the compound 29a (3.8 g, 96%) as white powder.
Compound 30a: The compound 29a (1.5 g, 0.19 mmol) was dissolved in anhydrous CH₂C12 (15 mL) followed by the addition of TFA (10 mL). The mixture was stirred at room temperature for 4 h. Solvent was removed under vacuum as much as possible at <35° C. The residue was recrystallized from CH₂Cl₂/methyl tert-butyl ether (6.0 mL/45 mL) twice. The isolated product was dried under vacuum at 35° C. to yield the compound 30a (1.4 g, 95%) as white powder.
Compound 33a: To a stirred solution of compound 30a (1.9 g, 0.05 mmol) in anhydrous CH₂C12 (20 mL) at 0° C., DIPEA (0.16 mL, 0.9 mmol) was added followed by 5-maleimidovalericacid-NHS (62 mg, 0.14 mmol). The mixture was stirred at 0° C. for 1.5 h, then allowed to warm up slowly from 0° C. to room temperature. The reaction was stirred under argon overnight. Solvent was removed and the residue was recrystallized from CH₂Cl₂/methyl tert-butyl ether (6.0 mL/45 mL). The isolated solids were recrystallized again from MeCN/2-propanol (4.5 mL/70 mL). The residue was dried under vacuum to yield the compound 33a (1.8 g, 93%) as white powder.
Compound 34a: To a stirred solution of compound 33a (1.8 g, 0.045 mmol) in anhydrous CH₂Cl₂ (25 mL) at room temperature under argon, DBCO-NH₂ (38 mg, 0.14 mmol), EDCI (53 mg, 0.28 mmol) and HOBt (56 mg, 0.4 mmol) were added. The mixture was stirred at room temperature until full conversion was observed by HPLC. The solvent was removed and the residue was recrystallized from CH₂Cl₂/methyl tert-butyl ether (6.0 mL/45 mL). The isolated solids were recrystallized again from MeCN/2-propanol (4.5 mL/70 mL). The product was dried at 40° C. over 4 h under vacuum to give the compound 34a (1.6 g, 91%) as white powder.
Compound 35b: To a stirred solution of compound 34a (1.4 g, 0.035 mmol) in anhydrous CH₂Cl₂ (10 mL) and MeOH (10 mL), the compound 22a (124 mg, 0.045 mmol) was added. The mixture was stirred at room temperature overnight until full conversion was observed by HPLC. Solvent was removed and the residue was recrystallized from MeCN/2-propanol (6.0 mL/45 mL) twice. The residue was dried under vacuum to yield the compound 35b (1.2 g, 88%) as white powder.

### Example 1e. Preparation of 2Arm-40kmPEG-Lys(Pyrimidin)DBCO-(MMAE)₂ (Compound 35c, FIG. 7c)

Compound 33b: A solution of compound 30a (1.6 g, 0.04 mmol) in 16 mL of anhydrous CH2Cl2 was treated with DIPEA (0.13 mL, 0.8 mmol) at room temperature. The compound 6-(2-(methylsulfonyl)pyrimidin-5-yl)hex-5-ynoic acid (32 mg, 0.12 mmol) EDCI (46 mg, 0.24 mmol) and HOBt (48 mg, 0.36 mmol) were then added to the mixture. The resulting mixture was stirred at room temperature until full conversion was observed by HPLC. The solvent was removed and the residue was recrystallized from CH₂Cl₂/methyl tert-butyl ether (6.0 mL/45 mL). The isolated solids were recrystallized again from MeCN/2-propanol (4.5 mL/70 mL). The product was dried at 40° C. over 4 h under vacuum to give the compound 33b (1.5 g, 91%) as white powder.
Compound 34b: To a stirred solution of compound 33b (1.3 g, 0.033 mmol) in anhydrous CH₂Cl₂ (13 mL) at room temperature under argon, DBCO-NH₂ (27 mg, 0.1 mmol), EDCI (37 mg, 0.2 mmol) and HOBt (40 mg, 0.3 mmol) were added. The mixture was stirred at room temperature until full conversion was observed by HPLC. The solvent was removed and the residue was recrystallized from CH₂Cl₂/methyl tert-butyl ether (6.0 mL/45 mL). The isolated solids were recrystallized again from MeCN/2-propanol (4.5 mL/70 mL). The product was dried at 40° C. over 4 h under vacuum to give the compound 34b (1.2 g, 93%) as white powder.
Compound 35c: To a stirred solution of compound 34b (1.1 g, 0.028 mmol) in anhydrous CH₂Cl₂ (7 mL) and MeOH (7 mL), the compound 22a (99 mg, 0.036 mmol) was added. The mixture was stirred at room temperature overnight until full conversion was observed by HPLC. Solvent was removed, the residue was recrystallized from MeCN/2-propanol (6.0 mL/45 mL) twice and the isolated wet solids were dried under vacuum to yield the compound 35c (1.0 g, 91%) as white powder.

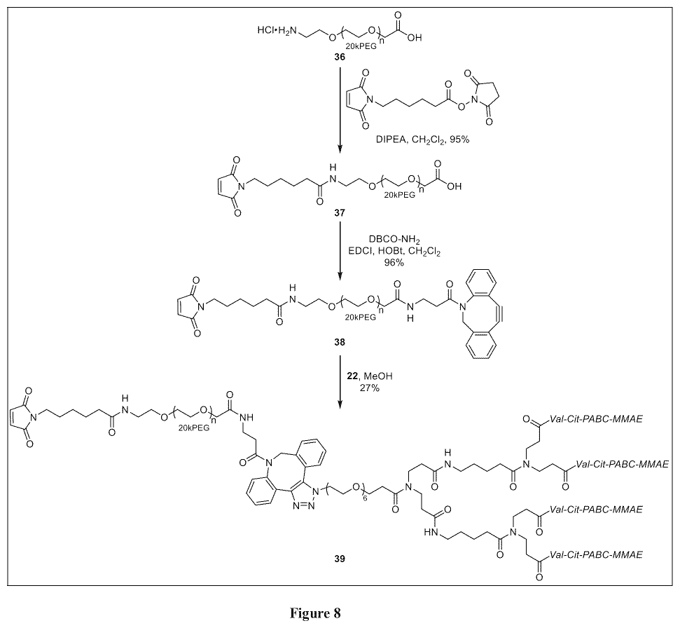

### Example 1f. Preparation of Mal-PEG(20K)-(MMAE)₄ (Compound 39, FIG. 8)

Compound 37: To a stirred solution of amine-PEG20k-CO₂H (36) (1.0 g, 0.05 mmol) in anhydrous CH₂Cl₂ (10 mL) at 0° C. was added DIPEA (83 μL, 0.5 mmol), followed by addition of 6-maleimidohexanoic acid-NHS (46 mg, 0.15 mmol). The mixture was stirred at 0° C. for 1.5 h, then the solution was allowed to warm up slowly from 0° C. to room temperature and then stirred under argon atmosphere overnight. Solvent was removed and the residue was recrystallized from CH₂Cl₂/methyl tert-butyl ether (2.5 mL/20 mL). The isolated solids were recrystallized again from MeCN/2-propanol (2 mL/30 mL). The residue was dried under vacuum to yield the compound 37 (0.95 g, 95%) as white powder.
Compound 38: To a stirred solution of compound 37 (0.9 g, 0.045 mmol) in anhydrous CH₂Cl₂ (9 mL) at room temperature under argon, DBCO-NH₂ (37 mg, 0.14 mmol), EDCI (52 mg, 0.27 mmol) and HOBt (55 mg, 0.41 mmol) were added. The mixture was stirred at room temperature until full conversion was observed by HPLC. The solvent was removed and the residue was recrystallized from CH₂Cl₂/methyl tert-butyl ether (2.5 mL/20 mL). The isolated solids were recrystallized again from MeCN/2-propanol (2 mL/30 mL). The product was dried at 40° C. over 4 h under vacuum to give the compound 38 (0.86 g, 89%) as white powder.
Compound 39: Compound 38 (166 mg, 0.008 mmol) was dissolved in anhydrous MeOH (3 mL), followed by addition of compound 22 (30 mg, 0.006 mmol). The mixture was stirred at room temperature for 24 h. The mixture was concentrated in vacuum and the crude reaction mixture was purified through preparative HPLC using Phenomenex Jupiter® C18 column (eluents: A=0.1% TFA in water, B=MeCN) to yield the compound 39 (37 mg, 27%) as white solid.

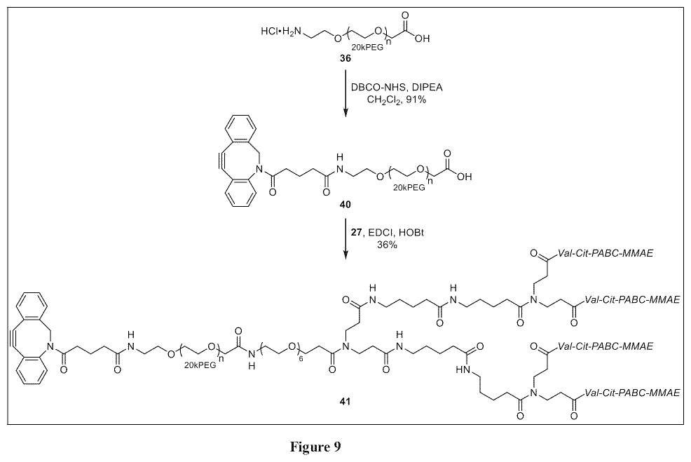

### Example 1g. Preparation of DBCO-PEG(20K)-(MMAE)₄ (Compound 41, FIG. 9)

Compound 40: To a stirred solution of amine-PEG20k-CO₂H (36) (1.0 g, 0.05 mmol) in anhydrous CH₂Cl₂ (10 mL) at 0° C. was added DIPEA (83 μL, 0.5 mmol), followed by addition of DBCO-NHS (60 mg, 0.15 mmol). The mixture was stirred at 0° C. for 1.5 h, then the solution was allowed to warm up slowly from 0° C. to room temperature and then stirred under argon atmosphere overnight. Solvent was removed and the residue was recrystallized from CH₂Cl₂/methyl tert-butyl ether (2.5 mL/20 mL). The isolated solids were recrystallized again from MeCN/2-propanol (2 mL/30 mL). The residue was dried under vacuum to yield the compound 40 (0.91 g, 91%) as white powder.
Compound 41: Under argon atmosphere, compound 40 (120 mg, 0.006 mmol) was dissolved in a mixture of CH₂Cl₂/DMF (2 mL/2 mL), then compound 27 (50 mg, 0.009 mmol), EDCI (6.9 mg, 0.036 mmol) and HOBt (7.3 mg, 0.054 mmol) was successively added to the above solution. The resulting reaction mixture was stirred at room temperature for 24 h. The mixture was concentrated in vacuum and the crude reaction mixture was purified through preparative HPLC using Phenomenex Jupiter® C18 column (eluents: A=0.1% TFA in water, B=MeCN) to yield the compound 41 (53 mg, 36%) as white solid.

### Example 2. Preparation of Bispecific Antibody SCACD47/SCAPD-L1

Bispecific single chain antibody (SCA) fragments of SCACD47 and SCAPD-L1 can be prepared via recombinant DNA technology in mammalian cells (e.g., CHO using Easy Select™) or yeast (e.g., Pichia pastori Expression Kit containing a pPICZ vector). DNA Sequence of SCACD47/SCAPD-L1 corresponding to amino acid sequence below (SEQ ID NO: 14) was synthesized and cloned into the expression vectors and transformed in the host cells. Expressed protein was purified by Ni-chelating resin or protein L resin. To facilitate the subsequent conjugation, a site specific conjugation functional group thiol was inserted through recombinant DNA technology into the linker between two SCAs.

Amino acid Sequence of SCACD47/SCAPD-L1:
(SEQ ID NO: 14)
DIVMTQSPLSLPVTPGEPASISCRSSQSIVYSNGNTYLGWYLQKPGQSPQ
LLIYKVSNRFSGVPDRFSGSGSGTDFTLKISRVEAEDVGVYYCFQGSHVP
YTFGQGTKLEIKGGSGGSGGSGGSGGQVQLVQSGAEVKKPGASVKVSCKA
SGYTFTNYNMHWVRQAPGQRLEWMGTIYPGNDDTSYNQKFKDRVTITADT
SASTAYMELSSLRSEDTAVYYCARGGYRAMDYWGQGTLVTVSSGCGGSSG
GSDIQMTQSPSSLSASVGDRVTITCRASQDVSTAVAWYQQKPGKAPKLLI
YSASFLYSGVPSRFSGSGSGTDFTLTISSLQPEDFATYYCQQYLYHPATF
GQGTKVEIKGGGGSGGGGSGGGGSEVQLVESGGGLVQPGGSLRLSCAASG
FTFSDSWIHWVRQAPGKGLEWVAWISPYGGSTYYADSVKGRFTISADTSK
NTAYLQMNSLRAEDTAVYYCARRHWPGGFDYWGQGTLVTHHHHHH;

in which the CDR sequences targeting CD47 are shown in bold and underlined and the CDR sequences targeting PD-L1 are shown in bold and italics.

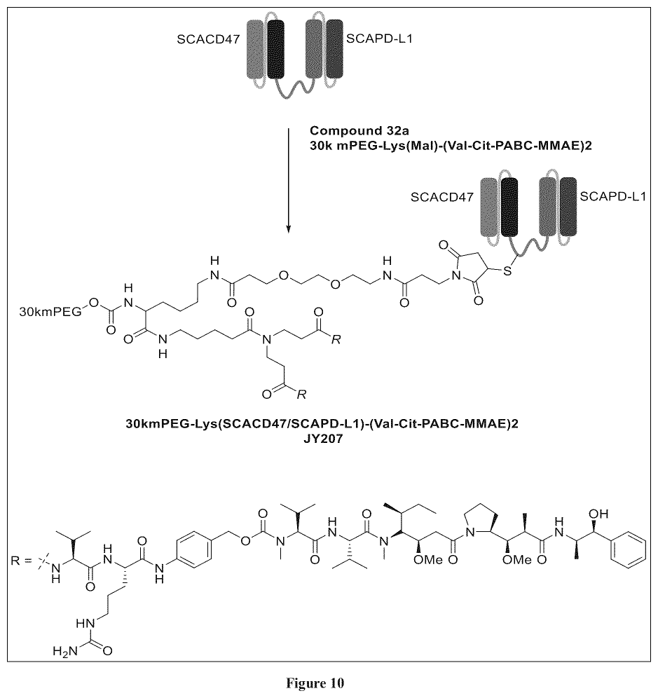

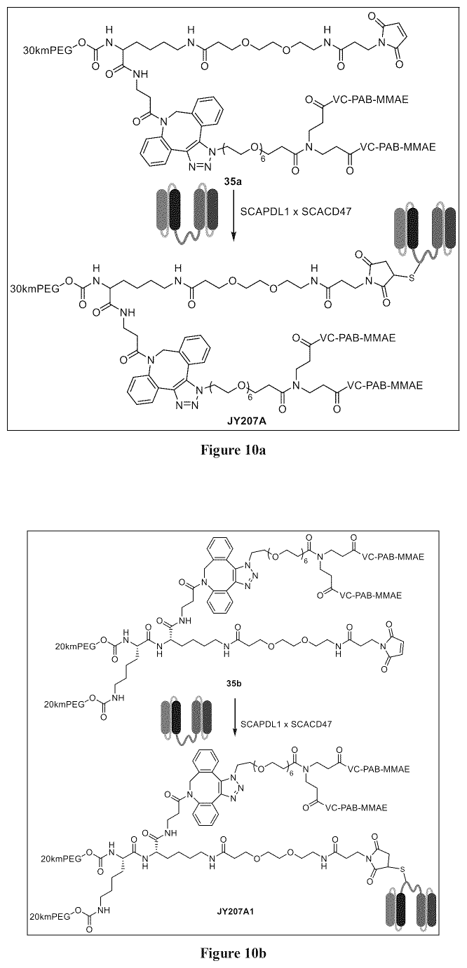

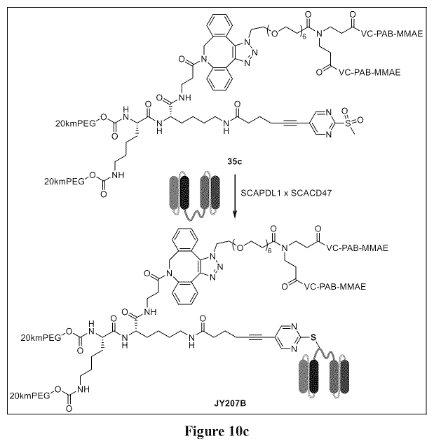

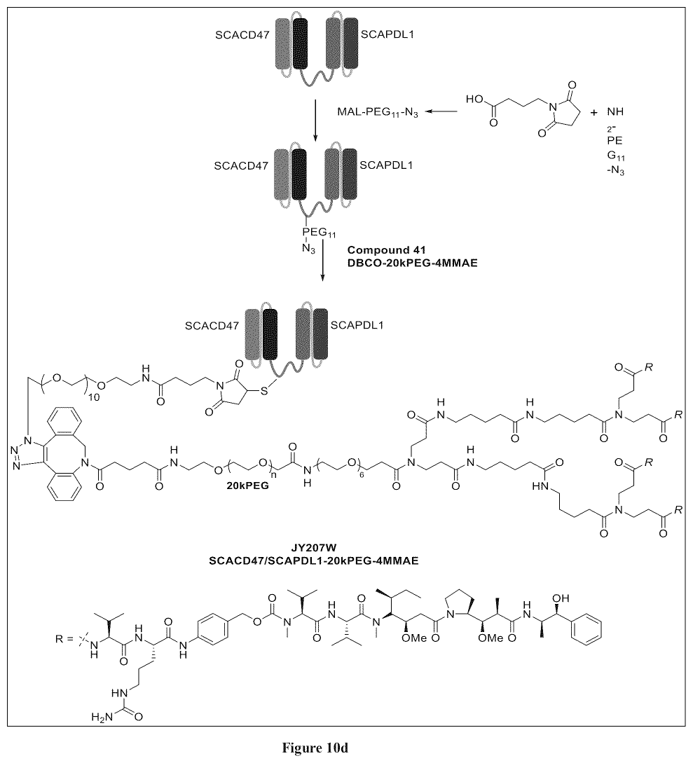

### Example 3. Preparation of JY207 (FIG. 10)

Compound JY207: Protein SCACD47/SCAPD-L1 was treated by reducing agent TCEP-HCl in PBS buffer (pH=7.4) at room temperature for 30 min before pH adjustment to pH 6.8 with a pH=4.12 stock solution of 500 mM sodium phosphate or a stock solution of 2M of Tris-HCl. TCEP-HCl was removed with an ultrafiltration and the treated protein was concentrated to 5 mg/mL before pegylation. Pegylation of SCACD47/SCAPD-L1 was conducted at room temperature for 3 hours with 1.5 to 5 mole equivalent of compound 32a [30k mPEG-Lys(Mal)-(Val-Cit-PABC-MMAE)₂. The reaction was quenched with 10 mM of L-cystine at room temperature for 10 min. Final product 30kmPEG-Lys(SCACD47/SCAPD-L1)-(Val-Cit-PABC-MMAE)₂ (JY207) was purified with a chromatographic column purification with Ca++ Pure-HA™ (Part #45039, TOSOH) first followed by a cation exchange chromatography column (CM Fast Flow). The target compound was confirmed by SEC-HPLC and cell-based activity assay.

### Example 3a. Preparation of JY207A (FIG. 10a)

Compound JY207A: Protein SCAPDL1/SCACD47 (20 mg) was treated by reducing agent 2 mM TCEP in PBS buffer (pH=7.4) at room temperature for 30 min before pH adjustment with a pH=4.12 stock solution of 500 mM sodium phosphate buffer. The treated protein was concentrated to 5 mg/mL before pegylation. Pegylation of SCAPDL1/SCACD47 was conducted at room temperature for 3 h with 2 to 3 equivalent of compound 35a. The reaction was quenched with 10 mM of L-cystine at room temperature for 10 min. Final product compound was purified with hydroxyapatite HA (TOSOH) at the pH 6.8 in 20 mM sodium phosphate buffer. The target compound JY207A was confirmed by SEC-HPLC and cell-based activity assays.

### Example 3b. Preparation of JY207A1 (FIG. 10b)

Compound JY207A1: Compound JY207A1 was made in the similar way as compound JY207A except using compound 35b instead of compound 35a.

### Example 3c. Preparation of JY207B (FIG. 10c)

Compound JY207B: Compound JY207B was made in the similar way as compound JY207A except using compound 35c instead of compound 35a.

### Example 4. Preparation of SCACD47/SCAPD-L1-20kPEG-4MMAE (JY207W) (FIG. 10d)

Compound Mal-PEG₁₁-N₃: 15 mg of N-succinimidyl-4-maleimidobutyrate (1.0 eq) was reacted with 38 mg of azido-dPEG₁₁-amine (1.2 eq) in 200 μL DMSO at room temperature for 30 min. Resulting compound azide-PEG₁₁-maleimide (Mal-PEG₁₁-N₃) was used immediately at next step without further purification.
Compound JY207W: Protein SCACD47/SCAPD-L1 was treated by reducing agent TCEP-HCl in PBS buffer (pH=7.4) at room temperature for 30 min before pH adjustment to pH 6.8 with a pH=4.12 stock solution of 500 mM sodium phosphate or a stock solution of 2M of Tris-HCl. TCEP-HCl was removed with an ultrafiltration and the treated protein was concentrated to 5 mg/mL. Fifty mole extra of Mal-PEG₁₁-N₃ was added to the reduced protein at room temperature while stirring. Protein-linker crude was passed through a chromatography column with Ca++ Pure-HA™ (Part #45039, TOSOH) and eluted off with a linear gradient, 0-50% buffer B in CVs, from Buffer A (5 mM phosphate pH6.8), to buffer Buffer B (200 mM phosphate, 200 mM arginine, pH6.8). Conjugation of protein-linker was conducted at room temperature for 3 hours with 1.5 to 5 mole equivalent of compound 41 (DBCO-20kPEG-4MMAE). Final product 20kmPEG-Lys(SCACD47/SCAPD-L1)-(Val-Cit-PABC-MMAE)₄ (JY207W) was purified with a chromatographic column purification with Ca++ Pure-HA™ (Part #45039, TOSOH) first followed by a cation exchange chromatography column (CM Fast Flow). The target compound was confirmed by SEC-HPLC and cell-based activity assay.

### Example 5. JY207 has No Cytotoxicity to Cell Lines Expressing Only CD47 or PD-L1

JY207 is a pegylated single chain bi-specific ADC targeting CD47 and PD-L1. We hypothesize that the JY207 is selective toward cell lines that express both PD-L1 and CD47. To prove this hypothesis, the cytotoxicity of JY207 to the cell lines with different expressing pattern of PD-L1 and CD47, including PD-L1⁺/CD47⁻ and PD-L1⁻/CD47⁺ cells, was measured.

To evaluate the cytotoxicity of JY207. Raji cells (Type Culture Collection of Chinese Academy of Science, Shanghai, China), MKN45 cells and NCIN87 cells (Procell, Wuhan, China) under standard maintenance were seeded in a 96-well plate at 3×10⁶ cells/well and treated with indicated doses of JY207, respectively. Cell viability (OD490) was measured after 3 days of treatment using an MTS (Promega Corporation, cat G3581) cell viability assay according to the manufacturer's instructions. Data were analyzed with GraphPad Prism software and presented as percent growth inhibition relative to the untreated control using following formula:

$$1 - ( OD control - ODJY 207 ) / OD control ) \times 100 \% .$$

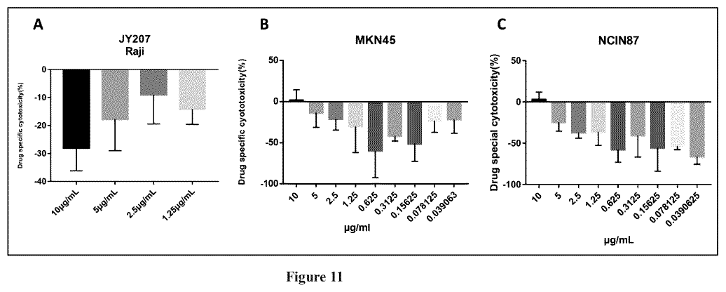

Among three single antigen positive cell lines, Raji is a leukemia cell line with PD-L1⁻ and CD47⁺. As we can see from FIG. 11, even with high concentration of 10 ug/ml, JY207 did not show any cytotoxicity. For the MKN45 cell line with very low PD-L1 expressing but CD47 positive, JY207 also did not induce cytotoxicity under almost all tested concentrations except for the highest concentration of 10 ug/ml, which induces 10% cytotoxicity. For the cell line NCIN87, which only expresses “physiological” levels of CD47 (similar to the level of CD47 of normal cells) and is regarded as CD47 negative and PD-L1 positive, JY207 again did not induce cytotoxicity except for the highest concentration of 10 ug/ml, which induces 10% cytotoxicity. The results shown in FIG. 11 demonstrated that JY207 did not induce cytotoxicity toward tumor cells expressing only CD47 or PD-L1.

### Example 6. JY207 Induces Cytotoxicity to Tumor Cells Lines Expressing Both CD47 and PD-L1

A panel of human cancer cell lines of NCIH1975, NCIH661, U87, Calu6, NCIH520, SKBR-3, BxPC-3 (Procell, Wuhan, China), HCC827 (GuangZhou Jennio Biotech Co., Ltd China), JIMT-1 (Shanghai Model Organisms Center, Inc) expressing both CD47 and PD-L1 were used to further evaluate the cytotoxic activity of the JY207 using the same method as that in Example 5.

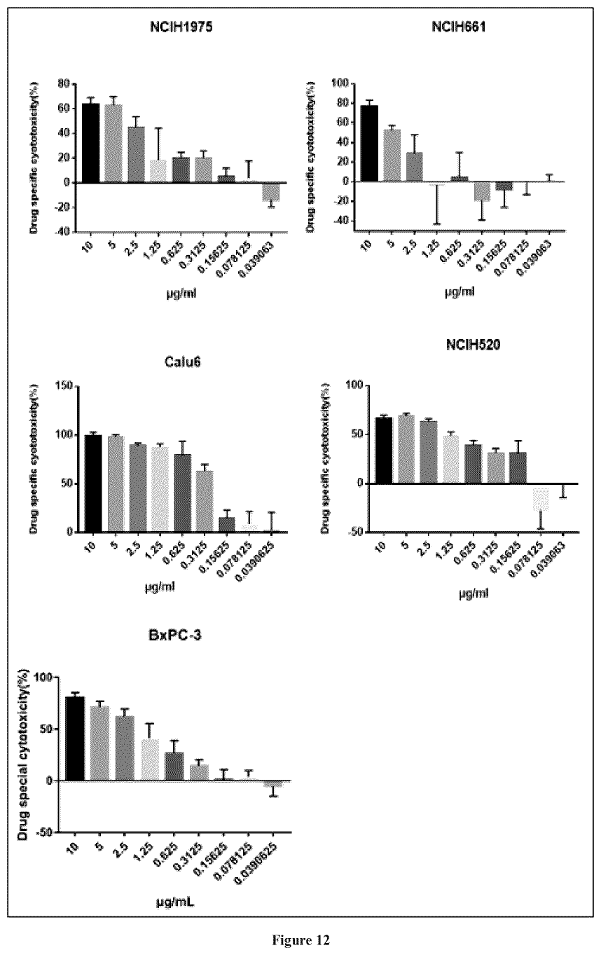

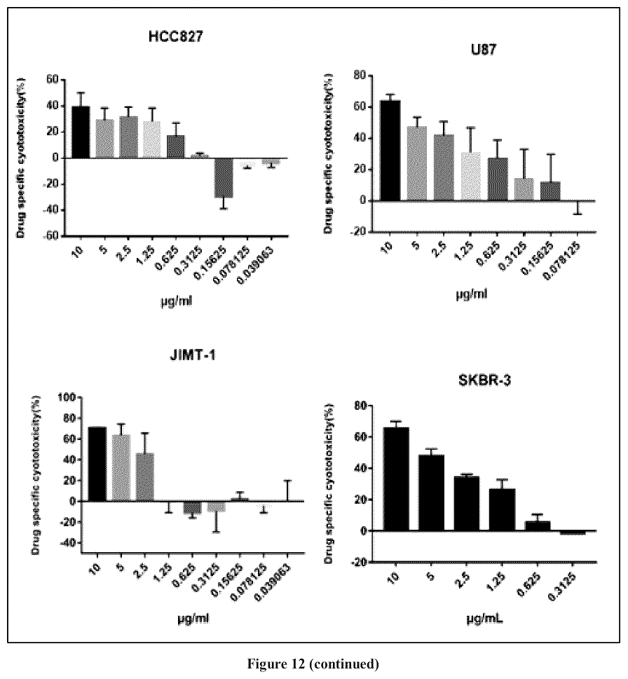

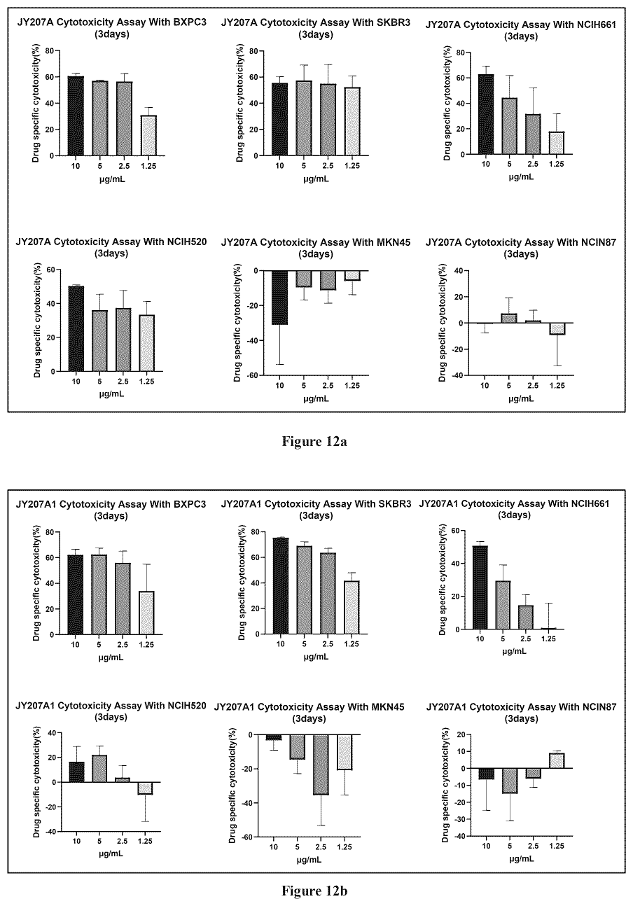

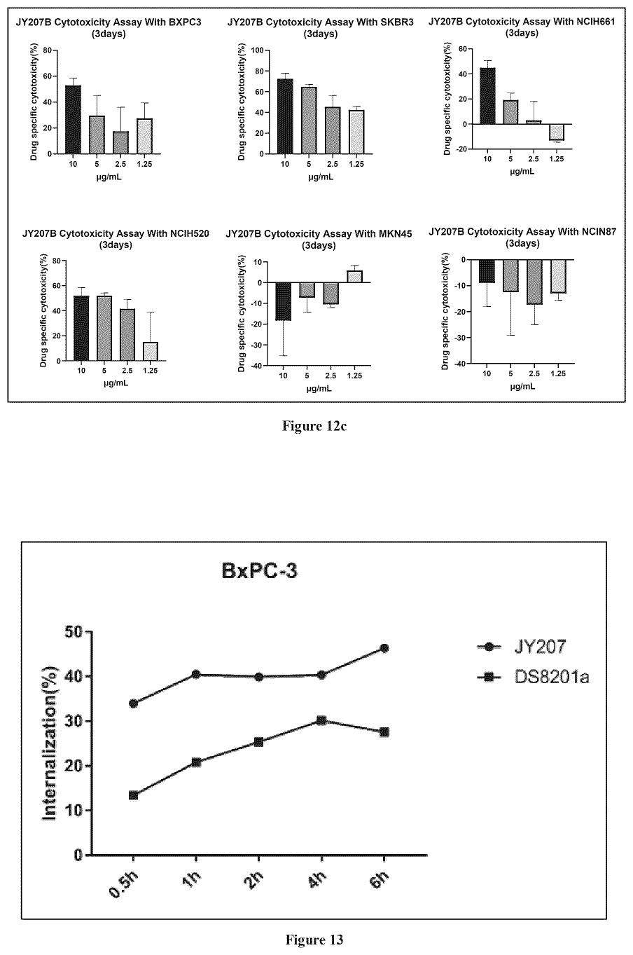

The results were presented in FIG. 12 as mean and SD (n=3) and demonstrated that JY207 could induce drug specific cytotoxicity to different CD47/PD-L1 double positive tumor cells. In general, the EC₅₀ of JY207 was found to be at μg/mL level.

### Example 6a. In Vitro Cytotoxicity of Compound JY207A, JY207A1 and JY207B to Tumor Cell Lines

To verify cytotoxic activity, CD47⁺/PD-L1⁺ tumor cell lines BXPC-3, SKBR-3, NCIH661 and NCIH520, as well as CD47⁺/PD-L1⁻ MKN45 and CD47⁻/PD-L1⁺ NCIN87 were selected for viability analysis in vitro. Cells were seeded in a 96-well plate at 3×10⁵ cells/well and treated with indicated doses of compound JY207A, JY207A1 or JY207B. Cell viability was determined by Cell Counting Kit-8 (CCK-8) according to the manufacturer's instructions. Data were analyzed with GraphPad Prism software and are presented as percent growth inhibition relative to the untreated control using the following equation: cytotoxicity %=(1−OD sample/OD control)×100%.

The results for JY207A, JY207A1 and JY207B were shown in FIGS. 12a, 12b and 12c, respectively. FIGS. 12a-12c showed that JY207A, JY207A1 and JY207B could induce drug specific cytotoxicity to different CD47/PD-L1 double positive tumor cells, and did not induce cytotoxicity toward tumor cells expressing only CD47 or PD-L1.

### Example 7. Internalization Induced by JY207

ADC's efficacy is dependent on the efficiency of antigen target mediated internalization to deliver the payload inside tumor cells. To examine the internalization rate of JY207, BxPC-3 cells were treated with 3 μg/mL Flour647 labeled DS8201a (Daiichi Sankyo, Product code: WJA0005) or JY207 at 4° C. overnight and then washed to remove unbound antibodies. An aliquot of cells remained at 4° C. and the rest were incubated at 37° C. for the indicated times to allow internalization. Cells were analyzed by flow cytometry and FlowJo software. Antigen-antibody complex internalization was calculated as percent mean fluorescent intensity (MFI) loss at 37° C. relatives to that at 4° C. after subtraction of background value of MFI derived from the untreated control.

Surprisingly, as shown in FIG. 13, JY207 elicited a much faster and higher level of receptor internalization than the approved ADC DS8201a.

### Example 8. JY207 In Vitro Selective Binding Assay

For all the CD47 related antibody drugs to be success, there is a requirement of minimizing or even eliminating on-target red blood cell (RBC) toxicity. This experiment will evaluate if JY207 will bind to red blood cell.

The fluorescence-activated cell sorting (FACS) method was used to detect the binding of JY207 to target cells. Briefly, freshly prepared red blood cells (from healthy donors) and BxPC-3 tumor cells (Procell, Wuhan, China) were used as target cells. The cells incubated with indicated doses of Flour647 labeled JY207 or FITC labeled Hu5F9-G4 (anti-CD47 antibody, expressed and prepared in house) for 30 min at 4° C. in the dark. The cells were washed at least twice with PBS and analyzed with flow cytometry after incubation.

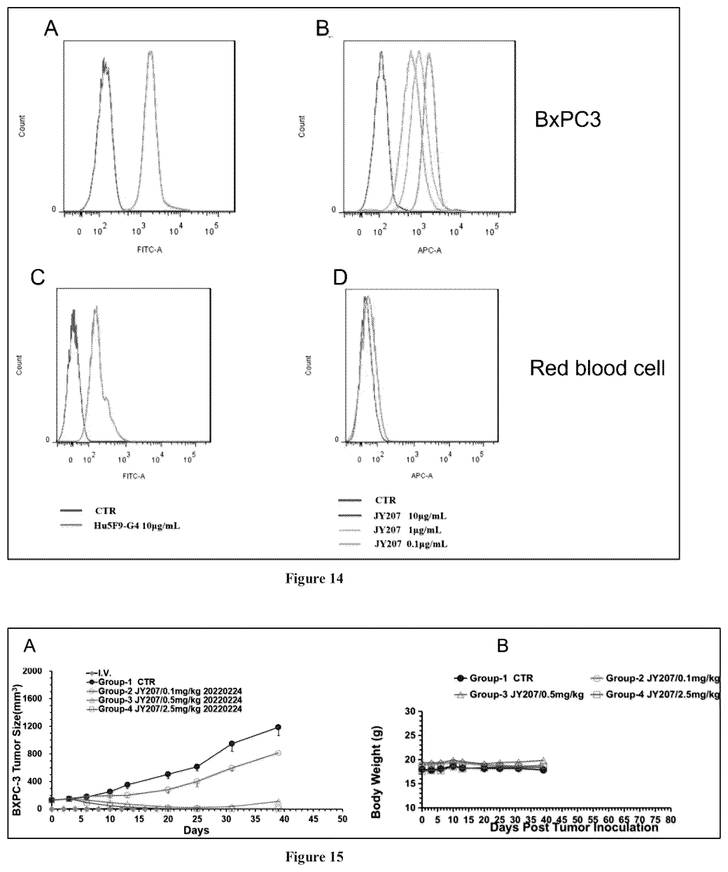

The results shown in FIG. 14 demonstrated that Hu5F9-G4 and JY207 could both specifically bind to BxPC-3. For the red blood cells, only Hu5F9-G4 binds with high affinity, while the binding of JY207 to RBCs from the same donor was undetectable. These data confirmed JY207 could selectively bind to tumor cells with both CD47⁺ and PD-L1⁺, but not to RBCs that is CD47⁺ and PD-L1⁻.

### Example 9. In Vivo Efficacy of JY207 on Pancreatic Cancer Xenograft Tumor Model

The in vivo antitumor activity of JY207 was evaluated in a CD47 and PD-L1 positive BxPC-3 xenograft model on NOD/SCID mice. BxPC-3 cells (2×10⁶) were injected subcutaneously into the back of the mice. When the average tumor volume reached approximately 100 mm³, the mice were randomly assigned to one of the four groups that received either a vehicle (as control) or JY207 (grouping and dosage schedule is shown in table 1, Q2D×10 refers to 10 doses given every other day). Tumor volume and body weight of the mice were measured twice a week or at indicated time points.

TABLE 1
Groups of Administration

| | No. of | | Dose of drug | Dosing |
|---|---|---|---|---|
| Group | Animals | Cells | (mg/kg) | cycle |
| Control (CTR) | 6 | BxPC-3 | PBS | Q2D × 10 |
| JY207 High dose group | 6 | BxPC-3 | 2.5 | |
| JY207 Middle dose group | 6 | BxPC-3 | 0.5 | |
| JY207 Low dose group | 6 | BxPC-3 | 0.1 | |

The results shown in FIG. 15 indicated that even at the low dose of 0.1 mg/kg, JY207 could significantly inhibit the growth of BxPC-3 xenograft tumors. As for the body weight, there was no significant difference between the mice in experimental groups and those in the control group. This suggested that JY207 at the experimental doses did not cause toxic effects on the animals.

### Example 10. In Vivo Efficacy of JY207 on NCI-H1975 Xenograft Tumor Model

The in vivo antitumor activity of JY207 was also evaluated in another CD47 and PD-L1 positive NCI-H1975 xenograft model on NOD/SCID mice. NCI-H1975 cells (2×10⁶) were injected subcutaneously into the back of the mice. When the average tumor volume reached approximately 100 mm³, the mice were randomly assigned to one of the two groups (n=6 for each group) that received either a vehicle (as control) or 3 mg/kg JY207 following the schedule of Q2D×10 (Q2D×10 refers to 10 doses given every other day). Tumor volume and body weight of the mice were determined periodically.

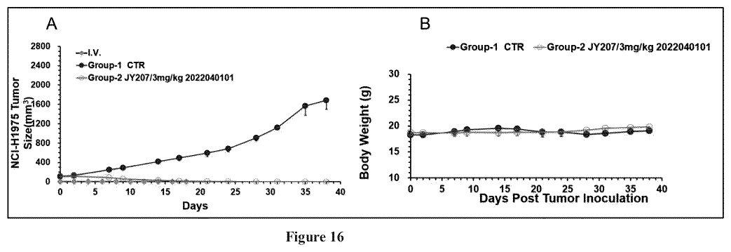

The results shown in FIG. 16 indicated that JY207 could effectively inhibit the growth of NCI-H1975 xenograft tumors at 3 mg/kg. As for the body weight, there was no significant difference between the mice in experimental groups and those in the control group. This suggested that JY207 at the experimental doses did not cause toxic effects on the animals.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims.

1. A compound of the Formula (I) 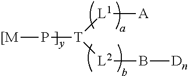
wherein
P is a non-immunogenic polymer;
M is a proton or a terminal capping group selected from C₁₋₅₀ alkyl and aryl, wherein one or more carbons of said alkyl are optionally replaced with a heteroatom;
y is an integer selected from 1 to 10;
A is a bispecific antibody or antigen binding fragment thereof targeting two different antigens selected from tumor specific antigens (TSA) and tumor associated antigens (TAA);
T is a trifunctional small molecule linker moiety;
each of L¹ and L² is independently a hetero or homobifunctional linker;
each of a and b is an integer selected from 0-10;
B is a branched linker, wherein each branch has an optional extension spacer, an amino acid sequence or carbohydrate moiety linked to a self-immolating spacer, wherein cleavage of the amino acid sequence or carbohydrate moiety by an enzyme triggers self-immolating mechanism to release D, or each branch has a disulfide bond, wherein cleavage of the disulfide bond releases D or its derivative, or each branch has a cleavable bond, wherein cleavage of the cleavable bond with certain cleavage mechanism releases D;
each of D is independently a cytotoxic small molecule or peptide; and
n is an integer selected from 1-25.

2. (canceled)

3. The compound of claim 1, wherein the functional group at the linker terminal of L¹ is capable of site-specific conjugation with A, and is selected from the group consisting of thiol, maleimide, methylsulfonyl pyrimidin, methylsulfonyl benzothiazole, vinylpyridine, ethyl P-ethynyl-N-(p-tolyl) phosphonamidate, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine; and
wherein both linkers L¹ and L² comprise linker chains that may be independently selected from
—(CH₂)ₐXY(CH₂)_{b}—,
—X(CH₂)ₐO(CH₂CH₂O)_{c}(CH₂)_{b}Y—,
-heterocyclyl-,
—(CH₂)ₐX—,
—X(CH₂)ₐY—,
—W₁—(CH₂)ₐC(O)NR₁(CH₂)_{b}O(CH₂CH₂O)_{c}(CH₂)_{d}X—,
—X(CH₂)ₐO(CH₂CH₂O)_{b}(CH₂)_{c}W₂C(O)(CH₂)_{d}Y—,
—W₃—(CH₂)ₐC(O)NR₁(CH₂)_{b}O(CH₂CH₂O)_{c}(CH₂)_{d}W₂C(O)(CH₂)ₑX—,
—C≡C—,
—CR₁═CR₂—,
wherein a, b, c, d and e are each an integer independently selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; each of X and Y is independently selected from C(═O), NR₂, S, O, CR₃R₄ or Null; R₁, R₂, R₃ and R₄ independently represent hydrogen, C1-10 alkyl or (CH₂)₁₋₁₀C(═O); W₁ and/or W₃ is derived from a maleimido-based moiety, methylsulfonyl pyrimidin-based moiety, methylsulfonyl benzothiazole-based moiety, 4-vinylpyridine-based moiety, ethyl P-ethynyl-N-phenylphosphonamidate-based moiety; W₂ represents a triazolyl or a tetrazolyl containing group; the heterocyclyl group is selected from a maleimido-derived moiety or a tetrazolyl-based or a triazolyl-based moiety,
and 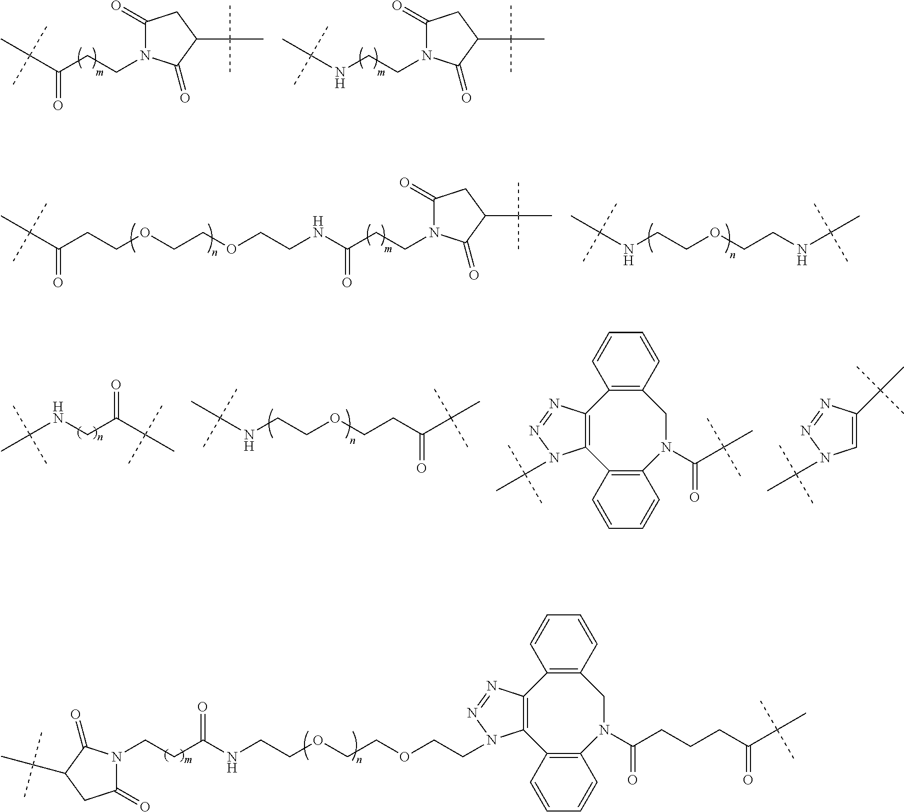
wherein n and m are integer and independently selected from 0 to 20.

4. The compound of claim 1, wherein the bispecific antibody is a single chain bispecific antibody, a bispecific nanobody, or a bispecific antigen binding domain thereof,
alternatively, wherein the bispecific antibody comprises an antigen-binding domain binding to CD47 comprising a light chain variable region (VL) and a heavy chain variable region (VH) and an antigen-binding domain binding to PD-L1 comprising a VL and a VH;
alternatively, wherein the bispecific antibody is a single chain anti-CD47/anti-PD-L1 bispecific antibody;
alternatively, wherein the VL of the antigen-binding domain binding to CD47 comprises CDR1, CDR2 and CDR3 as shown in SEQ ID Nos. 2-4 respectively, and the VH of the antigen-binding domain binding to CD47 comprises CDR1, CDR2 and CDR3 as shown in SEQ ID Nos. 5-7 respectively; and the VL of the antigen-binding domain binding to PD-L1 comprises CDR1, CDR2 and CDR3 as shown in SEQ ID Nos. 8-10 respectively, and the VH of the antigen-binding domain binding to PD-L1 comprises CDR1, CDR2 and CDR3 as shown in SEQ ID Nos. 11-13 respectively;
alternatively, wherein the VL of the antigen-binding domain binding to CD47 comprises an amino acid sequence as shown in SEQ ID No. 15 and the VH of the antigen-binding domain binding to CD47 comprises an amino acid sequence as shown in SEQ ID No. 16; and the VL of the antigen-binding domain binding to PD-L1 comprises an amino acid sequence as shown in SEQ ID No. 18 and the VH of the antigen-binding domain binding to PD-L1 comprises an amino acid sequence as shown in SEQ ID No. 17;
alternatively, wherein the bispecific antibody has an amino acid sequence as shown in SEQ ID NO: 1 or 14;
alternatively, wherein the antigen-binding domain binding to CD47 and the antigen-binding domain binding to PD-L1 are linked via a peptide linker or other chemical linker, and wherein the linker comprises a cysteine, an azide or an unnatural amino acid residue for site-specific conjugation of the bispecific antibody to L¹.

5. (canceled)

6. (canceled)

7. (canceled)

8. (canceled)

9. (canceled)

10. (canceled)

11. The compound of claim 1, wherein D is selected from any DNA crosslinker agent, microtubule inhibitor, DNA alkylator, topoisomerase inhibitor or a combination thereof,
alternatively, wherein D is selected from MMAE, MMAF, SN38, DM1, DM4, calicheamycins, pyrrolobenzodiazepines, duocarmycins or a derivate thereof, or a combination thereof; or wherein D is Dxd.

12. (canceled)

13. The compound of claim 1, wherein the non-immunogenic polymer is polyethylene glycol (PEG),
alternatively, wherein the PEG is a liner PEG or a branched PEG;
alternatively, wherein at least one terminal of the polyethylene glycol is capped with methyl or a low molecule weight alkyl;
alternatively, wherein a total molecule weight of the PEG is from 3000 to 100000;
alternatively, wherein the PEG is linked to T through a permanent bond or a cleavable bond.

14. (canceled)

15. (canceled)

16. (canceled)

17. (canceled)

18. A compound of the Formula (II) 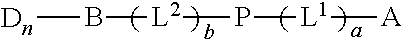
wherein
P is a liner PEG;
A is a bispecific antibody or antigen binding fragment thereof targeting two different tumor specific antigens (TSA) or tumor associated antigens (TAA);
each of L¹ and L² is independently a bifunctional linker;
each of a and b is an integer selected from 0-10;
B is a branched linker, wherein each branch has an optional extension spacer, an amino acid sequence or carbohydrate moiety linked to a self-immolating spacer, wherein cleavage of the amino acid sequence or carbohydrate moiety by an enzyme triggers self-immolating mechanism to release D, or each branch has a disulfide bond, wherein cleavage of the disulfide bond releases D or its derivative, or each branch has a cleavable bond, wherein cleavage of the cleavable bond with certain cleavage mechanism releases D;
each of D is independently a cytotoxic small molecule or peptide;
n is an integer selected from 1-25.

19. (canceled)

20. The compound of claim 18, wherein the functional group at the linker terminal of L¹ is capable of site-specific conjugation with A, and is selected from the group consisting of thiol, maleimide, methylsulfonyl pyrimidin, methylsulfonyl benzothiazole, vinylpyridine, ethyl P-ethynyl-N-(p-tolyl) phosphonamidate, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, boronic acid and Iodine; and
wherein both linkers L¹ and L² comprise linker chains that may be independently selected from
—(CH₂)ₐXY(CH₂)_{b}—,
—X(CH₂)ₐO(CH₂CH₂O)_{c}(CH₂)_{b}Y—,
-heterocyclyl-,
—(CH₂)ₐX—,
—X(CH₂)ₐY—,
—W₁—(CH₂)ₐC(O)NR₁(CH₂)_{b}O(CH₂CH₂O)_{c}(CH₂)_{d}X—,
—X(CH₂)ₐO(CH₂CH₂O)_{b}(CH₂)_{c}W₂C(O)(CH₂)_{d}Y—,
—W₃—(CH₂)ₐC(O)NR₁(CH₂)_{b}O(CH₂CH₂O)_{c}(CH₂)_{d}W₂C(O)(CH₂)ₑX—,
—C≡C—,
—CR₁═CR₂—,
wherein a, b, c, d and e are each an integer independently selected from 0 to 25, e.g. 0-20, 0-15, 0-10, 0-5, 5-25, 5-20, 5-15, 5-10, 10-25, 10-20, 10-15, 15-25, 15-20 or 20-25, e.g. 0, 1, 2, 3, 4, 5, 6, 7, 8, 9, 10, 11, 12, 13, 14, 15, 16, 17, 18, 19, 20, 21, 22, 23, 24, 25; each of X and Y is independently selected from C(═O), NR₂, S, O, CR₃R₄ or Null; R₁, R₂, R₃ and R₄ independently represent hydrogen, C1-10 alkyl or (CH₂)₁₋₁₀C(═O); W₁ and/or W₃ is derived from a maleimido-based moiety, methylsulfonyl pyrimidin-based moiety, methylsulfonyl benzothiazole-based moiety, 4-vinylpyridine-based moiety, ethyl P-ethynyl-N-phenylphosphonamidate-based moiety; W₂ represents a triazolyl or a tetrazolyl containing group; the heterocyclyl group is selected from a maleimido-derived moiety or a tetrazolyl-based or a triazolyl-based moiety,
and 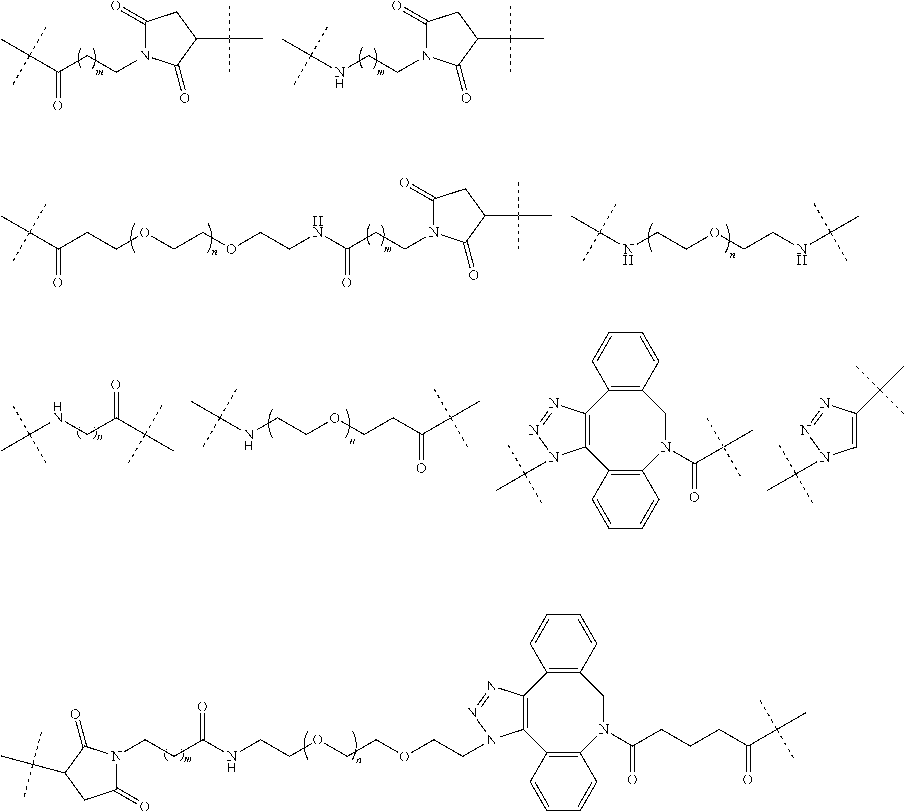
wherein n and m are integer and independently selected from 0 to 20.

21. The compound of claim 18, wherein the bispecific antibody is a single chain bispecific antibody, a bispecific nanobody, or a bispecific antigen binding domain thereof;
alternatively, wherein the bispecific antibody comprises an antigen-binding domain binding to CD47 comprising a VL and a VH and an antigen-binding domain binding to PD-L1 comprising a VL and a VH;
alternatively, wherein the bispecific antibody is a single chain anti-CD47/anti-PD-L1 bispecific antibody;
alternatively, wherein the VL of the antigen-binding domain binding to CD47 comprises CDR1, CDR2 and CDR3 as shown in SEQ ID Nos. 2-4 respectively, and the VH of the antigen-binding domain binding to CD47 comprises CDR1, CDR2 and CDR3 as shown in SEQ ID Nos. 5-7 respectively; and the V_{L} of the antigen-binding domain binding to PD-L1 comprises CDR1, CDR2 and CDR3 as shown in SEQ ID Nos. 8-10 respectively, and the VH of the antigen-binding domain binding to PD-L1 comprises CDR1, CDR2 and CDR3 as shown in SEQ ID Nos. 11-13 respectively;
alternatively, wherein the VL of the antigen-binding domain binding to CD47 comprises an amino acid sequence as shown in SEQ ID No. 15 and the VH of the antigen-binding domain binding to CD47 comprises an amino acid sequence as shown in SEQ ID No. 16; and the VL of the antigen-binding domain binding to PD-L1 comprises an amino acid sequence as shown in SEQ ID No. 18 and the VH of the antigen-binding domain binding to PD-L1 comprises an amino acid sequence as shown in SEQ ID No. 171;
alternatively, wherein the bispecific antibody has an amino acid sequence as shown in SEQ ID NO: 1 or 14;
alternatively, wherein the antigen-binding domain binding to CD47 and the antigen-binding domain binding to PD-L1 are linked via a peptide linker or other chemical linker, and wherein the linker comprises a cysteine, an azide or an unnatural amino acid residue for site-specific conjugation of the bispecific antibody to L¹.

22. (canceled)

23. (canceled)

24. (canceled)

25. (canceled)

26. (canceled)

27. (canceled)

28. The compound of claim 18, wherein D is selected from any DNA crosslinker agent, Microtubule inhibitor, DNA alkylator, Topoisomerase inhibitor or a combination thereof;
alternatively, wherein D is selected from MMAE, MMAF, SN38, DM1, DM4, calicheamycins, pyrrolobenzodiazepines, duocarmycins or a derivate thereof, or a combination thereof; or wherein D is Dxd.

29. (canceled)

30. The compound of claim 18, wherein a total molecule weight of the PEG is from 3000 to 100000;
alternatively, wherein the PEG is linked to L¹ and L² through a permanent bond or a cleavable bond.

31. (canceled)

32. The compound of claim 1 selected from the formula: 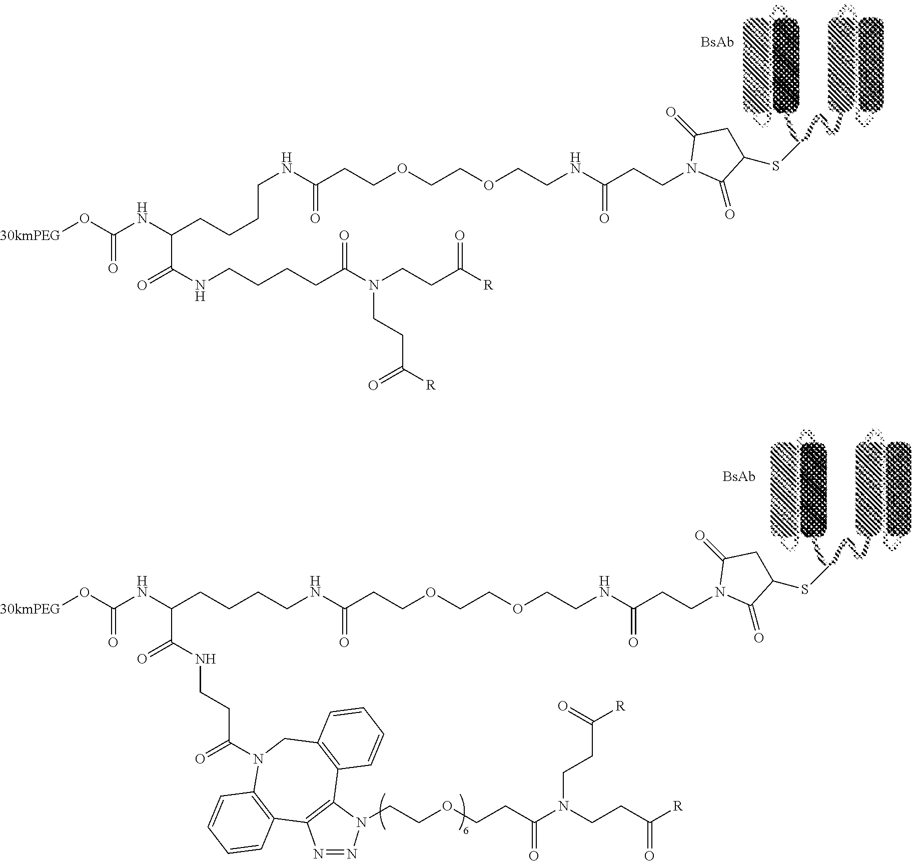 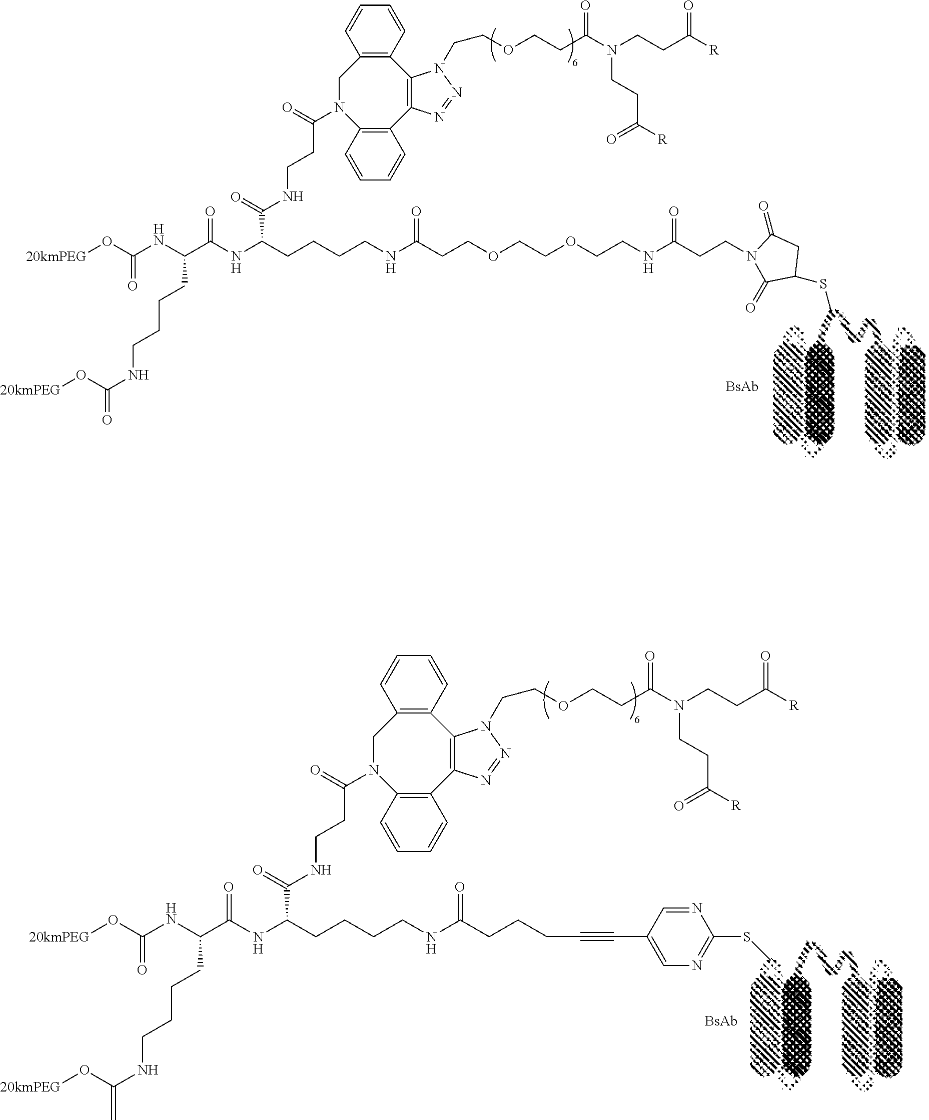 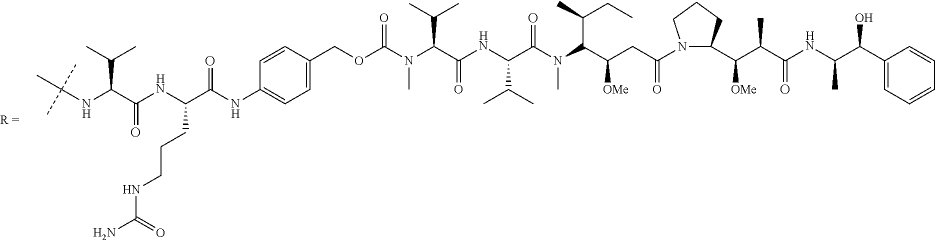
or a pharmaceutically acceptable salt thereof; wherein BsAb is a bispecific antibody targeting PD-L1 and CD47 or an antigen binding fragment thereof,
alternatively, wherein the bispecific antibody comprises an antigen-binding domain binding to CD47 comprising a VL comprising CDR1, CDR2 and CDR3 as shown in SEQ ID Nos. 2-4 respectively, and a VH comprising CDR1, CDR2 and CDR3 as shown in SEQ ID Nos. 5-7 respectively; and an antigen-binding domain binding to PD-L1 comprising a VL comprising CDR1, CDR2 and CDR3 as shown in SEQ ID Nos. 8-10 respectively, and a VH comprising CDR1, CDR2 and CDR3 as shown in SEQ ID Nos. 11-13 respectively;
alternatively, wherein the VL of the antigen-binding domain binding to CD47 comprises an amino acid sequence as shown in SEQ ID No. 15 and the VH of the antigen-binding domain binding to CD47 comprises an amino acid sequence as shown in SEQ ID No. 16; and the VL of the antigen-binding domain binding to PD-L1 comprises an amino acid sequence as shown in SEQ ID No. 18 and the VH of the antigen-binding domain binding to PD-L1 comprises an amino acid sequence as shown in SEQ ID No. 17;
alternatively, wherein the bispecific antibody has an amino acid sequence as shown in SEQ ID No: 1 or 14.

33. (canceled)

34. (canceled)

35. (canceled)

36. (canceled)

37. A pharmaceutical formulation comprising an effective amount of the compound of claim 1 and a pharmaceutically acceptable salt, carrier or excipient.

38. A method for treatment of a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer, wherein the method comprises administering an effective amount of the compound of claim 1 to a subject.

39. (canceled)

40. The compound of claim 18, which has the formula: 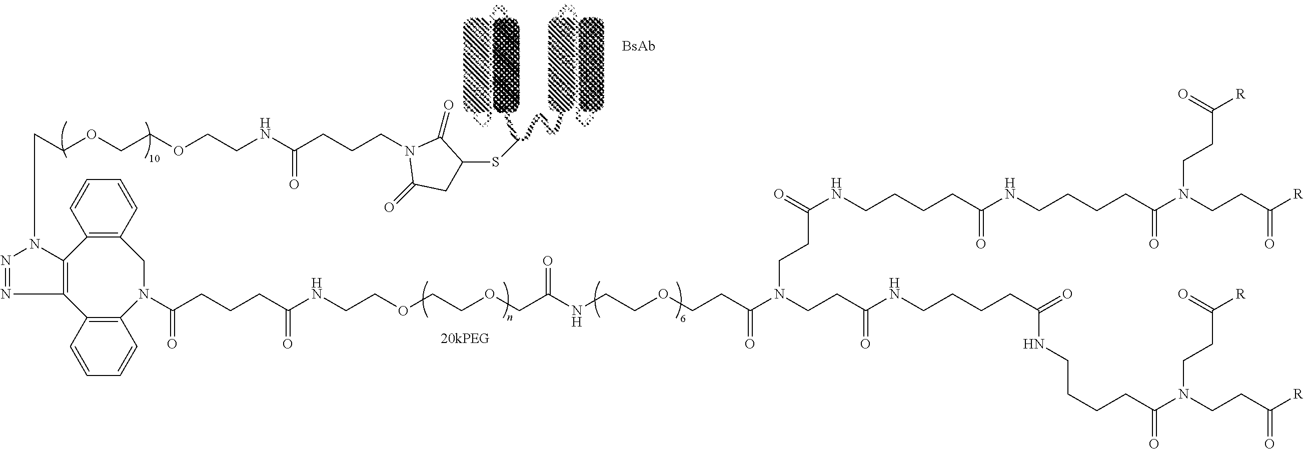 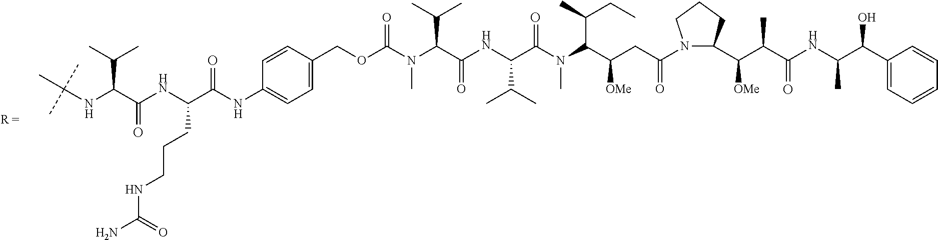
or a pharmaceutically acceptable salt thereof; wherein BsAb is a bispecific antibody targeting PD-L1 and CD47 or an antigen binding fragment thereof;
alternatively, wherein the bispecific antibody comprises an antigen-binding domain binding to CD47 comprising a VL comprising CDR1, CDR2 and CDR3 as shown in SEQ ID Nos. 2-4 respectively, and a VH comprising CDR1, CDR2 and CDR3 as shown in SEQ ID Nos. 5-7 respectively; and an antigen-binding domain binding to PD-L1 comprising a VL comprising CDR1, CDR2 and CDR3 as shown in SEQ ID Nos. 8-10 respectively, and a VH comprising CDR1, CDR2 and CDR3 as shown in SEQ ID Nos. 11-13 respectively;
alternatively, wherein the VL of the antigen-binding domain binding to CD47 comprises an amino acid sequence as shown in SEQ ID No. 15 and the VH of the antigen-binding domain binding to CD47 comprises an amino acid sequence as shown in SEQ ID No. 16; and the VL of the antigen-binding domain binding to PD-L1 comprises an amino acid sequence as shown in SEQ ID No. 18 and the VH of the antigen-binding domain binding to PD-L1 comprises an amino acid sequence as shown in SEQ ID No. 17;
alternatively, wherein the bispecific antibody has an amino acid sequence as shown in SEQ ID No: 1 or 14.

41. A pharmaceutical formulation comprising an effective amount of the compound of claim 18 and a pharmaceutically acceptable salt, carrier or excipient.

42. A method for the treatment of a cancer selected from the group consisting of non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom macroglobulinemia, breast cancer, uterus cancer, cervix cancer, ovarian cancer, prostate cancer, lung cancer, pancreatic cancer, kidney cancer, bladder cancer, stomach cancer, colon cancer, colorectal cancer, salivary gland cancer, thyroid cancer, skin cancers, bone cancer, brain cancer head and neck cancer and endometrial cancer, wherein the method comprises administering an effective amount of the compound of claim 18 to a subject.

43. The compound of claim 1, wherein T is derived from a molecule with three functional groups independently selected from hydroxyl, amino, hydrazinyl, carboxyl, thiol, and halide,
alternatively, wherein T is derived from lysine, aspartic acid, glutamic acid, 1,3-diamino-2-propanol, or triethanolamine.

44. The compound of claim 1, wherein the branched linker B is selected from; 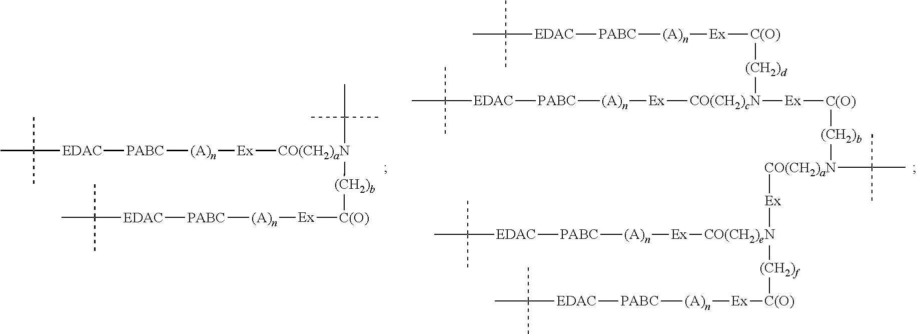 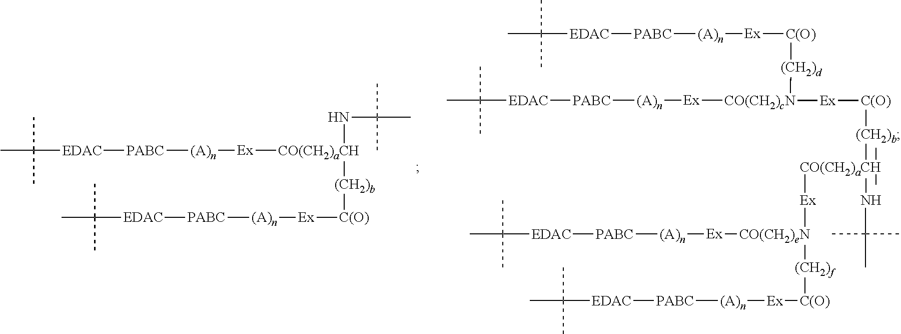 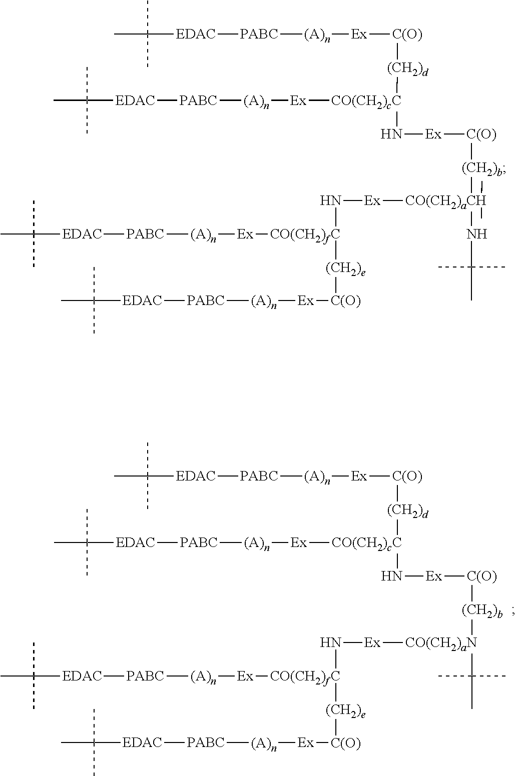 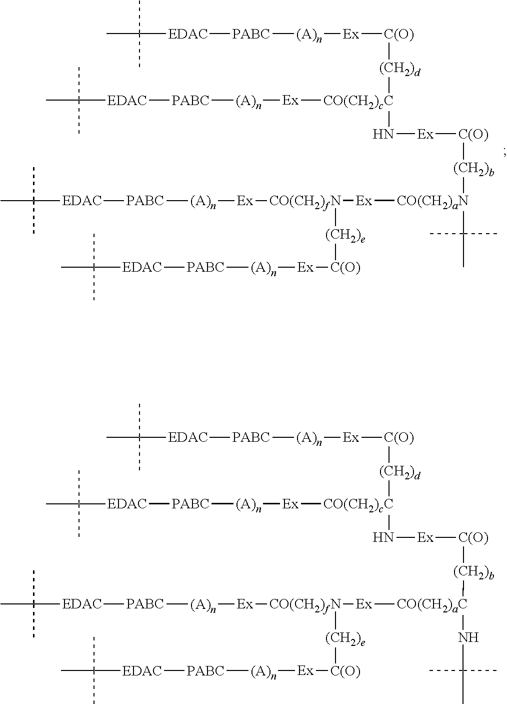 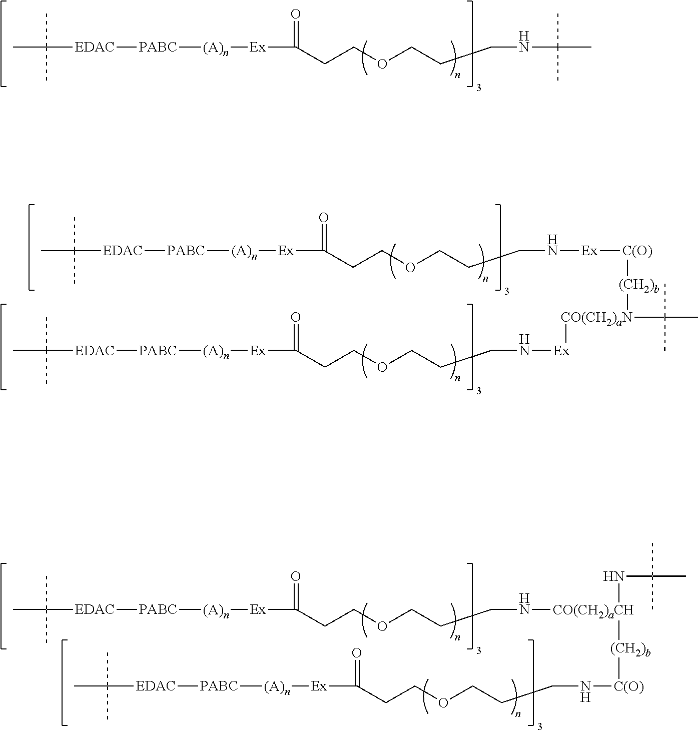
wherein:
each of a, b, c, d, e and f is independently an integer selected from 1-25;
n is an integer selected from 1 to 10;
(A)ₙ is a trigger unit of amino acid sequence, each A is an independent amino acid and n is any integer from 1-25;
PABC is 4-aminobenzyl alcohol carbonate;
EDAC is null or —CONR¹CH₂CH₂NR²— or —CONR¹CH₂CH₂CH₂NR²—, wherein R¹ and R² independently represent hydrogen, C₁₋₁₀ alkyl group or —(CH₂CH₂O)ₘCH₃, wherein m is any integer from 1-10;
Ex is an extension spacer that comprises linker chains that is independently selected from;
—NR¹(CH₂)ₐO(CH₂CH₂O)_{b}(CH₂)_{c}C(O)—,
—C(O)(CH₂)ₐNR¹—,
—NR¹(CH₂)ₐO(CH₂CH₂O)_{b}(CH₂)_{c}NR²—,
—NR¹(CH₂)ₐNR²—,
—NR¹(CH₂)ₐO(CH₂CH₂O)_{b}(CH₂)_{c}O—,
—O(CH₂)NR¹—,
—C(O)(CH₂)ₐO—,
—O(CH₂)ₐO(CH₂CH₂O)_{b}(CH₂)_{c}C(O)—,
—C(O)(CH₂)ₐO(CH₂CH₂O)_{b}(CH₂)_{c}C(O)—,
—C(O)(CH₂)ₐC(O)—,
or Null;
wherein a, b, and c are each an integer selected from 0 to 25, all subunits included; and R¹ and R² independently represent hydrogen or C₁₋₁₀ alkyl group.
alternatively, wherein branched linker B is selected from: 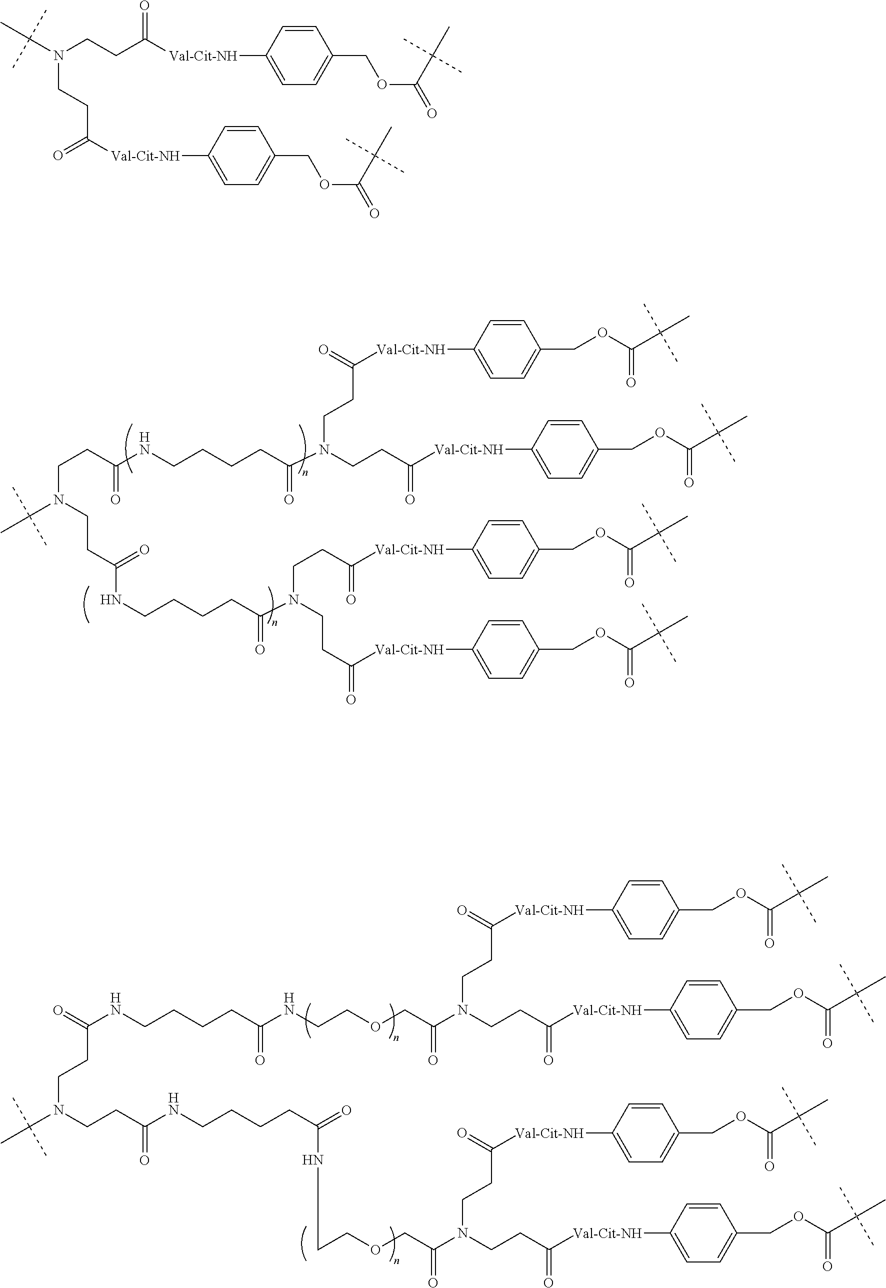 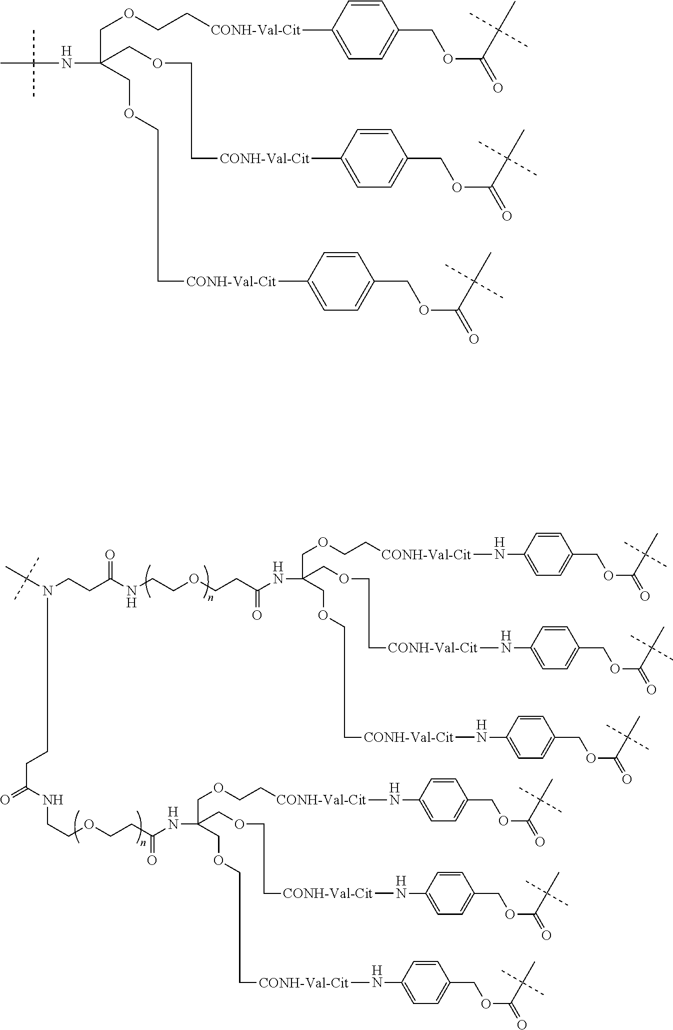
wherein n is an integer selected from 1 to 10.

45. The compound of claim 18, wherein the branched linker B is selected from: 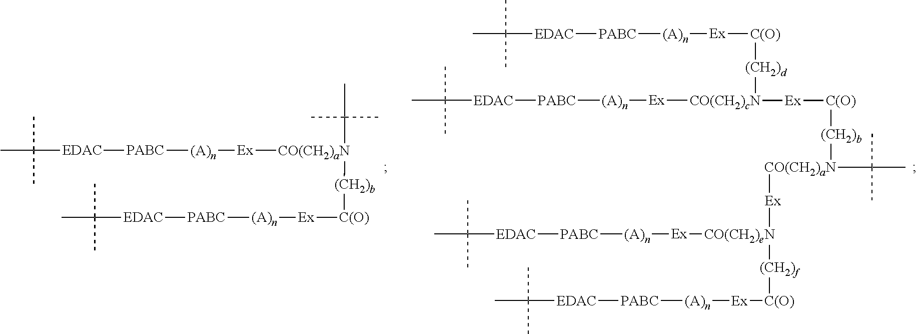 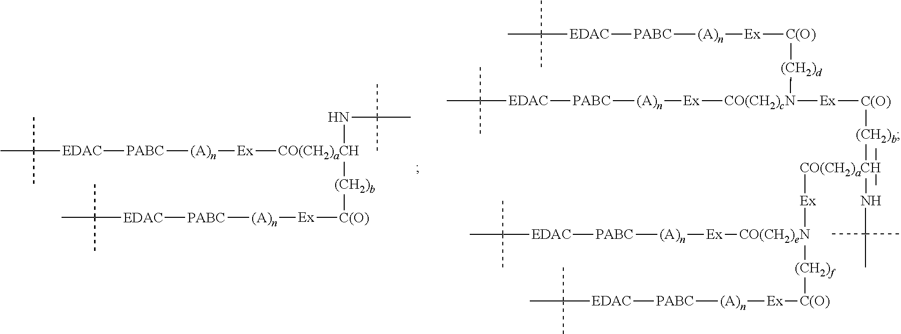 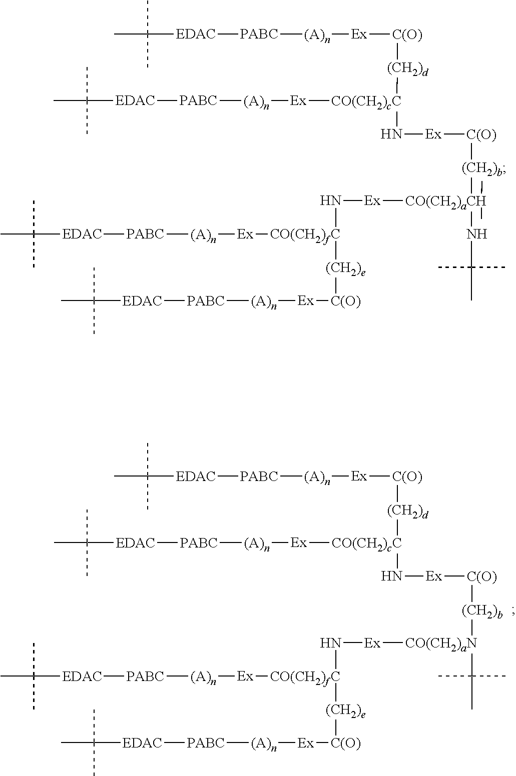 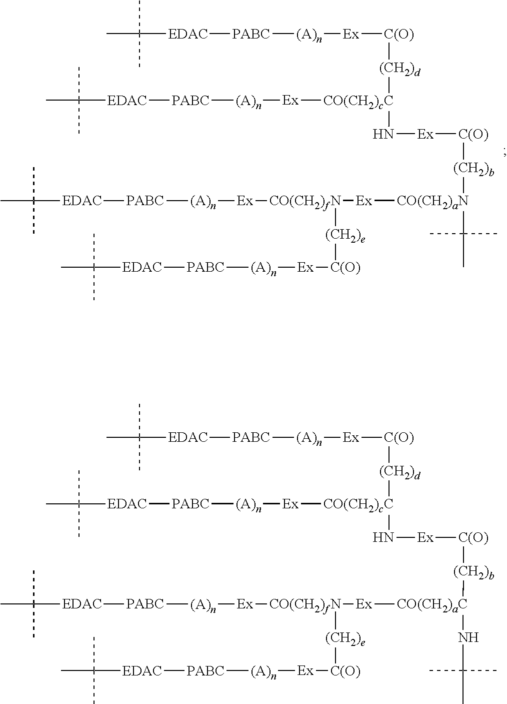 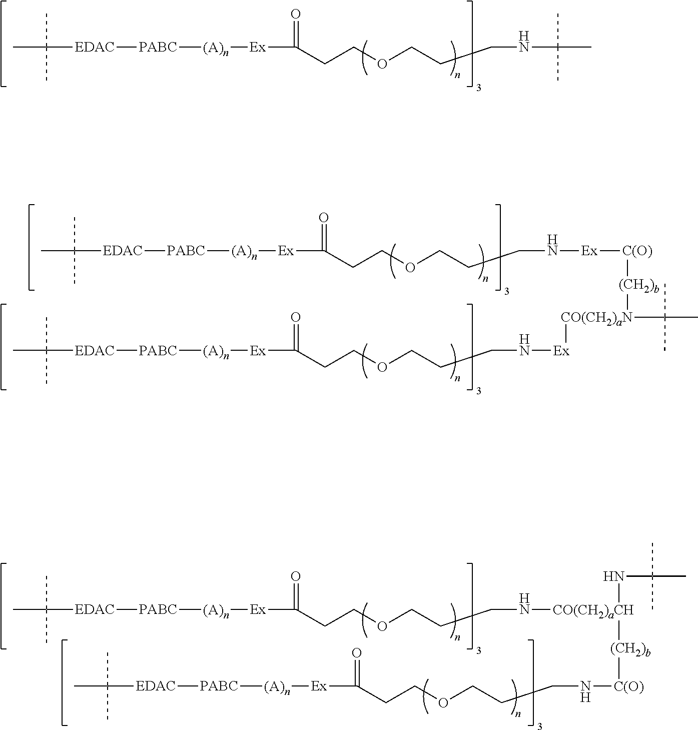
wherein:
each of a, b, c, d, e and f is independently an integer selected from 1-25;
n is an integer selected from 1 to 10;
(A)ₙ is a trigger unit of amino acid sequence, each A is an independent amino acid and n is any integer from 1-25;
PABC is 4-aminobenzyl alcohol carbonate;
EDAC is null or —CONR¹CH₂CH₂NR²— or —CONR¹CH₂CH₂CH₂NR²—, wherein R¹ and R² independently represent hydrogen, C₁₋₁₀ alkyl group or —(CH₂CH₂O)ₘCH₃, wherein m is any integer from 1-10;
Ex is an extension spacer that comprises linker chains that is independently selected from;
—NR¹(CH₂)ₐO(CH₂CH₂O)_{b}(CH₂)_{c}C(O)—,
—C(O)(CH₂)ₐNR—,
—NR¹(CH₂)ₐO(CH₂CH₂O)_{b}(CH₂)_{c}NR²,
—NR¹(CH₂)ₐNR²—,
—NR¹(CH₂)ₐO(CH₂CH₂O)_{b}(CH₂)cO—,
—O(CH₂)ₐNR¹—,
—C(O)(CH₂)ₐO—,
—O(CH₂)ₐO(CH₂CH₂O)_{b}(CH₂)_{c}C(O)—,
—C(O)(CH₂)ₐO(CH₂CH₂O)_{b}(CH₂)_{c}C(O)—,
—C(O)(CH₂)ₐC(O)—,
or Null;
wherein a, b, and c are each an integer selected from 0 to 25, all subunits included; and R¹ and R² independently represent hydrogen or C₁₋₁₀ alkyl group.
alternatively, wherein branched linker B is selected from; 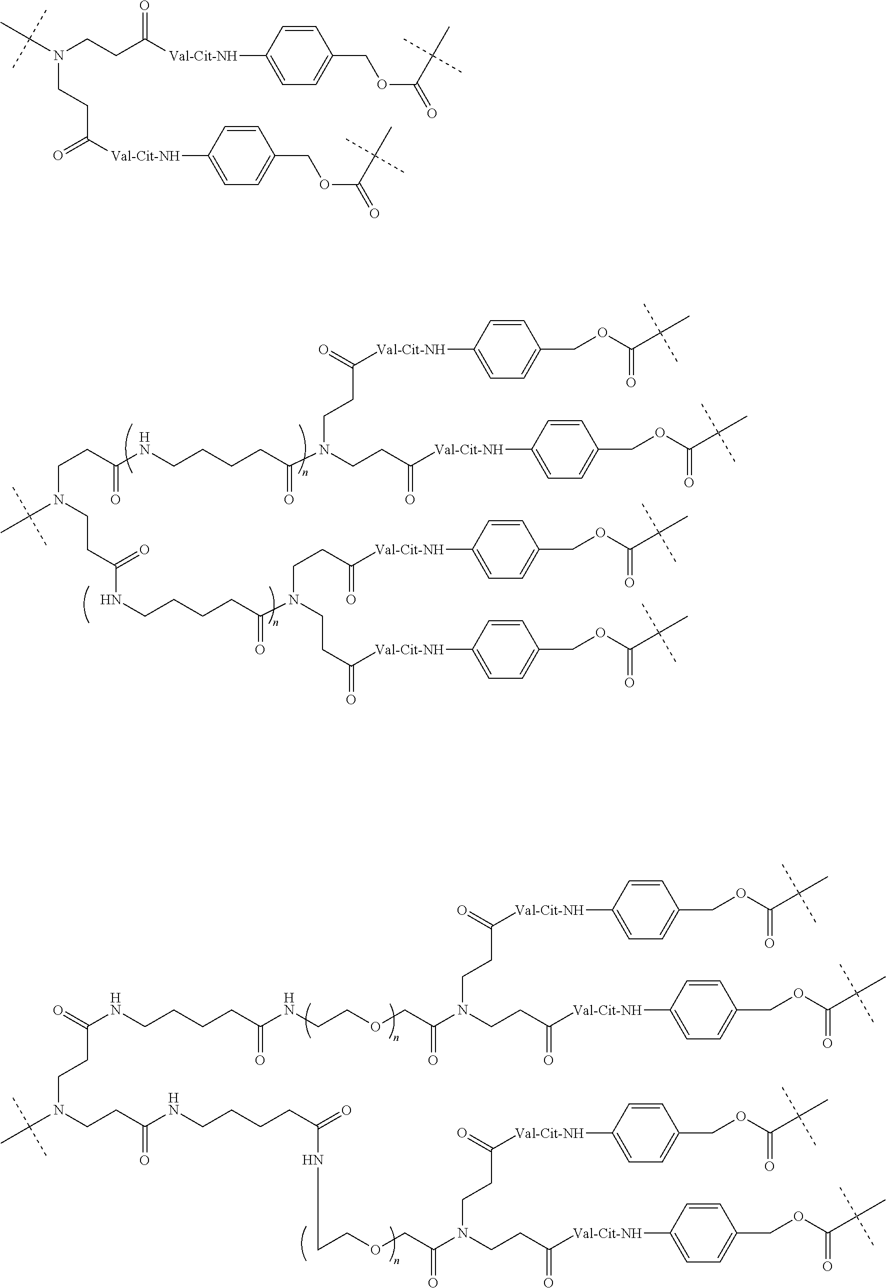 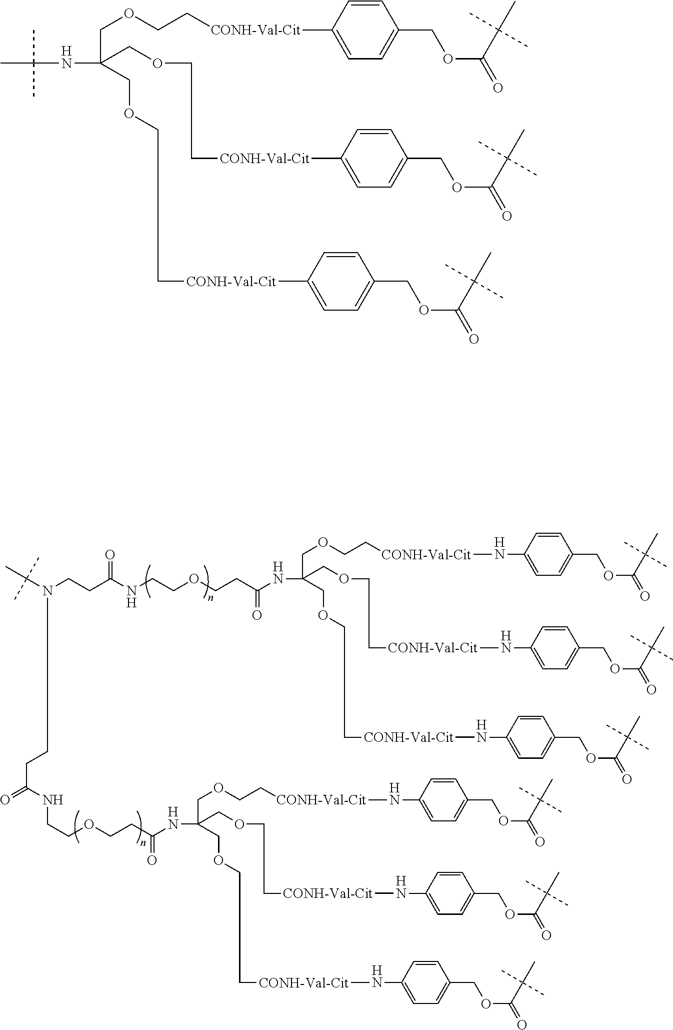
wherein n is an integer selected from 1 to 10.


SEQUENCE LISTING (18 sequences)

SEQ ID NO: 1 - protein, 489 aa, synthetic construct
  REGION 1..489: note = Amino acid sequence of SCAPD-L1 SCACD47
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKGGSGGSGGSGGSGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGCGGSSGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVT

SEQ ID NO: 2 - protein, 12 aa, synthetic construct
  REGION 1..12: note = CD47 LCDR1
RSSQSIVYSNGN

SEQ ID NO: 3 - protein, 7 aa, synthetic construct
  REGION 1..7: note = CD47 LCDR2
KVSNRFS

SEQ ID NO: 4 - protein, 9 aa, synthetic construct
  REGION 1..9: note = CD47 LCDR3
FQGSHVPYT

SEQ ID NO: 5 - protein, 5 aa, synthetic construct
  REGION 1..5: note = CD47 HCDR1
NYNMH

SEQ ID NO: 6 - protein, 16 aa, synthetic construct
  REGION 1..16: note = CD47 HCDR2
TIYPGNDDTSYNQKFK

SEQ ID NO: 7 - protein, 8 aa, synthetic construct
  REGION 1..8: note = CD47 HCDR3
GGYRAMDY

SEQ ID NO: 8 - protein, 10 aa, synthetic construct
  REGION 1..10: note = PD-L1 LCDR1
GFTFSDSWIH

SEQ ID NO: 9 - protein, 18 aa, synthetic construct
  REGION 1..18: note = PD-L1 LCDR2
AWISPYGGSTYYADSVKG

SEQ ID NO: 10 - protein, 9 aa, synthetic construct
  REGION 1..9: note = PD-L1 LCDR3
RHWPGGFDY

SEQ ID NO: 11 - protein, 11 aa, synthetic construct
  REGION 1..11: note = PD-L1 HCDR1
RASQDVSTAVA

SEQ ID NO: 12 - protein, 7 aa, synthetic construct
  REGION 1..7: note = PD-L1 HCDR2
SASFLYS

SEQ ID NO: 13 - protein, 9 aa, synthetic construct
  REGION 1..9: note = PD-L1 HCDR3
QQYLYHPAT

SEQ ID NO: 14 - protein, 495 aa, synthetic construct
  REGION 1..495: note = Amino acid sequence of SCAPD-L1 SCACD47
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKGGSGGSGGSGGSGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGCGGSSGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTHHHHHH

SEQ ID NO: 15 - protein, 112 aa, synthetic construct
  REGION 1..112: note = VL targeting CD47
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 16 - protein, 117 aa, synthetic construct
  REGION 1..117: note = VH targeting CD47
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 17 - protein, 107 aa, synthetic construct [sequence omitted]

SEQ ID NO: 18 - protein, 115 aa, synthetic construct
  REGION 1..115: note = VL targeting PD-L1
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVT